SEQUENCE LISTING (19 sequences)

SEQ ID NO: 1 - protein, 6 aa, artificial sequence
  completely synthesized
XYXPML

SEQ ID NO: 2 - protein, 4 aa, artificial sequence
  completely synthesized
GQPR

SEQ ID NO: 3 - protein, 4 aa, artificial sequence
  completely synthesized
XQPR

SEQ ID NO: 4 - protein, 4 aa, artificial sequence
  completely synthesized
AQTR

SEQ ID NO: 5 - protein, 4 aa, artificial sequence
  completely synthesized
GQRP

SEQ ID NO: 6 - protein, 4 aa, artificial sequence
  completely synthesized
VVRP

SEQ ID NO: 7 - protein, 5 aa, artificial sequence
  completely synthesized
KTTKS

SEQ ID NO: 8 - protein, 5 aa, artificial sequence
  completely synthesized
XTTKS

SEQ ID NO: 9 - protein, 5 aa, artificial sequence
  completely synthesized
XGGFM

SEQ ID NO: 10 - protein, 5 aa, artificial sequence
  completely synthesized
VVRPP

SEQ ID NO: 11 - protein, 5 aa, artificial sequence
  completely synthesized
XGGFL

SEQ ID NO: 12 - protein, 5 aa, artificial sequence
  completely synthesized
GPFPL

SEQ ID NO: 13 - protein, 5 aa, artificial sequence
  completely synthesized
XPFPL

SEQ ID NO: 14 - protein, 6 aa, artificial sequence
  completely synthesized
VGVAPG

SEQ ID NO: 15 - protein, 6 aa, artificial sequence
  completely synthesized
XGVAPG

SEQ ID NO: 16 - protein, 6 aa, artificial sequence
  completely synthesized
ARHLFW

SEQ ID NO: 17 - protein, 6 aa, artificial sequence
  completely synthesized
ADLKPT

SEQ ID NO: 18 - protein, 6 aa, artificial sequence
  completely synthesized
VVRPPP

SEQ ID NO: 19 - protein, 6 aa, artificial sequence
  completely synthesized
ARHXXW

(19) United States
(12) Patent Application Publication
(10) Pub. No.: US 2010/0047296 A1
(43) Pub. Date: Feb. 25, 2010

(54) Low-Residue Cosmetic Or Dermatological Stick Based On An Oil-In-Water Dispersion/Emulsion III

(75) Inventors: Bernhard Banowski, Duesseldorf (DE); Marcus Claas, Hilden (DE); Nadine Buse, Hilden (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/465,983

(22) Filed: May 14, 2009

Related U.S. Application Data
(63) Continuation of application No. PCT/EP2007/062236, filed on Nov. 13, 2007.

(30) Foreign Application Priority Data
Nov. 14, 2006 (DE) 10 2006 053 886.2

(51) Int. Cl. A61K 8/04; A61K 8/27; A61K 8/28; A61Q 15/00

(52) U.S. Cl. 424/401; 424/67; 424/66

(57) ABSTRACT

Cosmetic or dermatological sticks, especially deodorant or antiperspirant sticks, based on an oil-in-water dispersion/emulsion to be applied to the skin.

### CROSS REFERENCE TO RELATED APPLICATIONS

This application is a continuation under 35 U.S.C. §§120 and 365(c) of International Application PCT/EP2007/062236, filed on Nov. 13, 2007. This application also claims priority under 35 U.S.C. §119 of DE 10 2006 053 886.2, filed on Nov. 14, 2006. The disclosures of PCT/EP2007/062236 and DE 10 2006 053 886.2 are incorporated herein by reference in their entirety.

### BACKGROUND OF THE INVENTION

The invention relates to cosmetic or dermatological stick compositions, in particular deodorant or antiperspirant sticks, based on an oil-in-water dispersion/emulsion for the application of cosmetic or dermatological, in particular water-soluble active ingredients to the skin.

Standard commercial deodorants and antiperspirants are mostly formulated as sprays or as sticks; there are also roll-on preparations and creams in the market. Many stick antiperspirant preparations are formulated as anhydrous suspension sticks. Preparations of this type leave behind a pleasant dry feel on the skin for the user following application. However, effective release of the water-soluble antiperspirant active ingredients from such preparations is limited, and in most cases the feeling of freshness valued by many consumers is not realized. The anhydrous preparations, in particular those based on volatile silicone oils, have the disadvantage that the dispersed active ingredients readily lead to visible product residues on skin and clothing. Furthermore, such preparations are relatively expensive since the oil components are more expensive as active ingredient carriers than water. Compression during application often results in the loss of oil, which reduces the cosmetic acceptance of these preparations for the user.

Compared with, anhydrous sticks, as are known, for example, from U.S. Pat. No. 5,733,534 and WO 00/67713 A1, emulsion sticks, as are disclosed, for example, in WO 98/17238 A1, U.S. Pat. No. 4,814,165, DE 2 335 549, U.S. Pat. No. 4,725,431, U.S. Pat. No. 5,466,457 and U.S. Pat. No. 4,948,578, have a number of advantages. Replacing the wax and oil additives with water makes the emulsion sticks more cost-effective to manufacture. The emulsified waxes convey a soft, gentle feel on the skin, and, finally, water-soluble cosmetic active ingredients (i.e. in particular, antiperspirant active ingredients) can more readily be released onto the skin since they are already present in dissolved form in the aqueous phase of the emulsion. US 20020051758 discloses antiperspirant sticks without a W/O emulsifier or high melting wax, which contain a siliconized polyamide as a consistency regulator or structurant. According to patent claim 1 of US 20020051758, the aqueous phase forms the internal phase, i.e. the dispersed phase, such that the disclosed gels are water-in-oil emulsions. US 20020072506 A1 discloses, with reference to several exemplifying embodiments, hydrous antiperspirant sticks based on a water-in-oil emulsion, which comprise acylated cellobiose as the consistency regulator or structurant as well as a high fraction of inventively unfavorable silicone and hydrocarbon oils, and furthermore neither comprise oil-in-water emulsifiers nor a high melting wax.

Since the emulsion sticks of the cited prior art are formulated on the basis of a water-in-oil dispersion/emulsion, the water-soluble active ingredients are present in the inner, dispersed phase and, following application, must first migrate through the outer, lipophilic layer in order to reach their site of action on the skin. The known water-in-oil emulsion sticks thus have disadvantages, which are similar to those of anhydrous suspension sticks with regard to the availability of active ingredient. U.S. Pat. No. 6,428,776 discloses water-containing and oil-containing, wax-free antiperspirant sticks based on an oil-in-water emulsion. Sticks of this type have inadequate cosmetic properties, leave behind unpleasant sticky and visible residues and exhibit a stability that is insufficient for prolonged use. One example with glycerol monostearate as the W/O emulsifier and octyldodecanol as the oil component has a medium-firm consistency and a greasy feel on the skin and already begins to soften at 50° C. WO 99/59537 A1 discloses hydrous cosmetic sticks, which comprise wax components with a melting point of >50° C., nonionic water-in-oil emulsifiers, a nonionic oil-in-water emulsifier with an HLB value of more than 7 and a polyol. Some of the sticks contain oils which are liquid at 25° C. but which, instead of being incorporated at the beginning of the emulsion process as in the sticks of the present application, are stirred in at a temperature of 55° C. as a pre-emulsified concentrate, for instance a micro-emulsion or PIT emulsion, during the cooling phase of the stick compound. This type of production method is needed in order not to endanger or even destroy the stability of the system of a dispersion of lipid and wax crystals. Sticks of this type likewise have inadequate cosmetic properties, can leave behind unpleasant sticky and visible residues, and exhibit a stability that is inadequate for prolonged use.

US 20030103921 A1 discloses structured antiperspirant compositions in the form of a microemulsion which represents an oil-in-water microemulsion or a water-in-oil microemulsion or a bicontinuous phase, depending on the type and quantity of surfactants, but in which the bicontinuous phase predominates overall. The (transparent) microemulsions are thickened by an oil-soluble or oil-dispersible “structurant.” The oil-soluble or oil-dispersible “structurant” is chosen from esters and amides of 12-hydroxystearic acid, esters and amides of N-acylamino acids, esters and amides of di- and tricarboxylic acids, sterols, sterol esters such as oryzanol, cellobiose fatty acid esters, sugar esters such as acylated maltose, and non-crosslinked oil-soluble or oil-dispersible polymeric oil phase thickening agents such as e.g. the commercial product Kraton G. Non-ionic emulsifiers with an HLB value from 2-15, preferably with an HLB value under 12, are also incorporated. Polyols are disclosed as only optional. This document does not disclose the possible significance of matching the solubility parameters of W/O emulsifiers and oil components to one another. The structural difference between these compositions and the oil-in-water dispersion/emulsion sticks of the present invention, which are not microemulsions, becomes particularly clear because of the high fraction, namely 19-66 wt. % relative to the overall composition, of inventively unfavorable silicone and (paraffinic) hydrocarbon oils which are disclosed in all exemplifying embodiments. The published applications DE 199 62 878 A1 and DE 199 62 881 A1 disclose deodorant or antiperspirant creams based on an oil-in-water emulsion which, at 21° C., have a viscosity of at least 50 000 mPas, preferably in the range from 200 000-1 500 000 mPas, i.e., they are in viscous to highly viscous paste form. These creams comprise wax components with a melting point of >50° C., nonionic water-in-oil emulsifiers, but neither an ethylene glycol ester nor a pentaerythrityl ester, nonionic oil-in-water emulsifiers with an HLB value of more than 7, and a polyol. Being soft creams, they can be applied either by using only the fingers, which is rejected by many consumers as being impractical, or by pouring the creams into special applicators, which are significantly more expensive than the stick sheaths for the deodorant or antiperspirant sticks according to the invention. If, after being heated and mixed, the compositions disclosed in DE 199 62 878 A1 and DE 199 62 881 A1 were cooled statically, i.e. without stirring, then stick-like compositions would be obtained which have overall unfavorable application properties, such as poor haptics and/or inadequate stability, for example as a result of phase separation or the formation of water condensation, since the emulsifiers and the oils are not matched to one another as in the present invention. US 20060029624 A1 discloses deodorant or antiperspirant sticks in the form of an oil-in-water dispersion containing at least one lipid or wax component with a melting point of >50° C., at least one non-ionic oil-in-water emulsifier with an HLB value above 7 within an oil-in-water emulsifier system with an average HLB value in the range from 10 and 19, at least one nonionic water-in-oil emulsifier with an HLB value of greater then 1.0 and less than/equal to 7.0, which can form liquid crystalline structures with water alone or with water in the presence of a hydrophilic emulsifier, and as a consistency regulator and/or water binder, at least one oil which is in a liquid state at 20° C. and is not a fragrance component or essential oil, wherein the maximum deviation between the (average) solubility parameter of all the constituent oils and the (average) solubility of the water-in-oil emulsifier or emulsifiers is −0.7 (cal/cm³)^{0.5} or +0.7 (cal/cm³)^{0.5} in the presence of linear saturated fatty alcohol as the water-in-oil emulsifier or part of a water-in-oil emulsifier, respectively, and −0.4 (cal/cm³)^{0.5} or +0.7 (cal/cm³)^{0.5} in the presence of water-in-oil emulsifiers other than linear saturated fatty alcohols in the absence of linear saturated fatty alcohols as a water-in-oil emulsifier, respectively; at least one water-soluble polyhydric C₂-C₉ alkanol with 2-6 hydroxyl groups and/or at least one water-soluble polyethylene glycol with 3-20 ethylene oxide units; 5% to less than 50 wt. % of water relative to the whole composition; and at least one deodorant or antiperspirant agent; wherein the stick exhibits a penetration force value in the range of 200-600 gram-force (g-force) at a depth of 5.000 mm (five millimetres) and a maximum electrical resistance of 300 kΩ (Kiloohm). The documents U.S. 60/788,022 resp. PCT/EP2006/004371, which are not prepublished, disclose deodorant or antiperspirant sticks in the form of an oil-in-water dispersion/emulsion containing at least one wax component with a melting point of >50° C., at least one non-ionic oil-in-water emulsifier with an HLB value above 7 within an oil-in-water emulsifier system with an average HLB value in the range from 10 and 19, at least one nonionic water-in-oil emulsifier with an HLB value of greater then 1.0 and less than/equal to 7.0, selected from the mono- and diesters of ethylene glycol and the mono-, di-, tri- and tetraesters of pentaerythritol with linear saturated and unsaturated fatty acids with 12 to 30, more preferred 14 to 22, carbon atoms and which can be hydroxylated, as well as mixtures thereof, as a consistency regulator and/or water binder, at least one oil which is in a liquid state at 20° C. and is not a fragrance component or essential oil, wherein the maximum deviation between the (average) solubility parameter of all the constituent oils and the (average) solubility of the water-in-oil emulsifier or emulsifiers is −0.7 (cal/cm³)^{0.5} or +0.7 (cal/cm³)^{0.5} in the presence of linear saturated fatty alcohol with a chain length of at least 8 carbon atoms, respectively, and −0.4 (cal/cm³)^{0.5} or +0.7 (cal/cm³)^{0.5} in the presence of water-in-oil emulsifiers other than linear saturated fatty alcohols with a chain length of at least 8 carbon atoms in the absence of linear saturated fatty alcohols with a chain length of at least 8 carbon atoms as a water-in-oil emulsifier, respectively; at least one water-soluble polyhydric C₂-C₉ alkanol with 2-6 hydroxyl groups and/or at least one water-soluble polyethylene glycol with 3-20 ethylene oxide units; 5% to less than 50 wt. % of water relative to the whole composition; and at least one deodorant or antiperspirant agent.

However, it has been found that the application behavior of the sticks disclosed in US 20060029624 A1 and U.S. 60/788,022 or PCT/EP2006/004371, particularly with regard to the lubricating effect on the skin, required improvement. It was furthermore determined that the haptic and stickiness of the sticks disclosed in US 20060029624 A1 and U.S. 60/788,022 or PCT/EP2006/004371 should be improved.

### DESCRIPTION OF THE INVENTION

Therefore, the object was to develop a deodorant or antiperspirant composition, which is suitable as an effective carrier for water-soluble active ingredients and permits the rapid release of the active ingredient on the skin. A further object was to develop a stick composition, in particular a deodorant or antiperspirant composition in stick form, with excellent cosmetic care properties. A further object was to develop a stick, in particular a deodorant or antiperspirant stick which, on the one hand, has high stability, i.e., solidity, but on the other hand, has a pleasant release behavior, i.e. is not too solid but can be readily spread over the skin and in so doing releases an adequate amount of product. A further object was to develop a stick composition, in particular a deodorant or antiperspirant composition in stick form which, when applied to the skin, leaves behind as little sticky or visible residue as possible. A further object was to develop a stick composition, in particular a deodorant or antiperspirant composition in stick form, which leaves behind optimally little visible residue on clothing that comes into contact with the treated skin. A further object was to develop a stick composition, in particular a deodorant or antiperspirant composition in stick form, which can be readily washed off of the skin.

A further object was to develop a stick composition, in particular a deodorant or antiperspirant composition in stick form, with a cost-performance ratio, which is favorable economically and in terms of application. A further object was to develop a stick composition, in particular a deodorant or antiperspirant composition in stick form, which allowed for the mass production of stable sticks having a suitable consistency.

Surprisingly and unforeseeably to the person skilled in the art, these objects were achieved through a cosmetic or dermatological stick in the form of an oil-in-water dispersion/emulsion, comprising
a) at least one wax component with a melting point of >50° C. which is not included in components b) or c),
b) at least one nonionic oil-in-water emulsifier with an HLB value of more than 7 within an oil-in-water emulsifier system with an average HLB value in the range of 11-17,
c) at least one nonionic water-in-oil emulsifier with an HLB value greater than 1.0 and less than or equal to 7.0, which solely with water or with water in the presence of a hydrophilic emulsifier can form liquid crystalline structures, as a consistency regulator and/or water binder,
d) at least one oil that is a liquid under normal conditions and is neither a fragrance component nor essential oil, wherein the maximum deviation between the (average) solubility parameter of the totality of the comprised oils and the (average) solubility parameter of the water-in-oil emulsifier/of the water-in-oil emulsifiers is −1.0 (cal/cm³)^{0.5} resp.+1.0 (cal/cm³)^{0.5},
e) at least one water-soluble polyhydric C₂-C₉ alkanol containing 2-6 hydroxyl groups and/or at least one water-soluble polyethylene glycol having 3-20 ethylene oxide units,
f) 5-70 wt. % water, relative to the total composition,
g) at least one cosmetic or dermatological active ingredient.

The wax component with a melting point of >50° C. forms a gel matrix with the oil(s) and optionally further higher-melting wax components; this gel matrix can absorb larger amounts of water and polyol. These structures, which are stabilized by certain amounts of water-in-oil emulsifiers and oil-in-water emulsifiers, leave behind a fresh, cooling impression upon application due to their water content. Here, the emulsifiers are matched to one another such that the stick compositions according to the invention are present in the form of an oil-in-water dispersion/emulsion. The stick compositions according to the invention are thus not present as a microemulsion. To produce the stick compositions of the invention, the water phase and the oil phase must be heated to at least 70° C., preferably to at least 80° C., particularly preferably 90-95° C. and stirred together or homogenized while hot, i.e. at least at 70° C., preferably at least 80° C., particularly preferably 90-95° C. in order to achieve the emulsion structure of the invention. A production method like the one disclosed in U.S. Pat. No. 4,205,062, for example (kneading of fat and water phase at 65° C.) is inadequate for obtaining a stick composition, particularly a homogenous stick composition, based on an oil-in-water dispersion/emulsion. Without wishing to be bound to this theory, it is assumed that the oil-in-water emulsifiers, together with some of the water-in-oil emulsifiers, form lamellar liquid crystal phases, which are built up with some of the water into a hydrophilic gel phase. This hydrophilic gel phase surrounds the aqueous bulk phase. Dispersed within this aqueous bulk phase are, in turn, the lipophilic components, surrounded by a lipophilic gel phase, which is formed by the water-in-oil emulsifiers with some of the oil-in-water emulsifiers and some water. Water-soluble cosmetic or dermatological active ingredients, such as in particular preferred antiperspirant active ingredients, are dissolved in the outer, continuous aqueous phase, resulting in a considerably improved and more efficient active ingredient release compared to the known anhydrous suspension sticks and water-in-oil emulsion sticks. The O/W emulsion basis of the stick compositions of the invention results in a considerably improved and more efficient active ingredient release compared to the known anhydrous suspension sticks and water-in-oil emulsion sticks. This active ingredient release can be determined indirectly very readily by measuring the electrical resistance of the particular product. Measuring the electrical resistance of such compositions is also a suitable way to be able to distinguish between an oil-in-water system and a water-in-oil system. An oil-in-water system exhibits a high electrical conductivity and therefore a low electrical resistance owing to the continuous water phase. The precise measurement set-up and the measurement procedure are described below (see below). The sticks according to the invention accordingly have an electrical resistance of preferably at most 400 kΩ, particularly preferably of at most 300 kΩ, and particularly preferably of at most 80 kW. In contrast, the sticks disclosed in WO 98/17238 A1 exhibit an electrical resistance of more than 3 000 kΩ; therefore, they obviously employ a water-in-oil system.

All statements concerning the state of aggregation of the used starting materials (solid, liquid . . . ) in this application refer to normal conditions. “Normal conditions” in the context of the present application are a temperature of 20° C. and a pressure of 1013.25 mbar.

Melting point data also refer to a pressure of 1013.25 mbar

The solidification of the sticks according to the invention, preferably to the deodorant or antiperspirant sticks according to the invention, does not take place on the basis of soap gels or fatty acid salt gels, fatty acids being understood as meaning alkanoic, alkenoic and alkynoic acids having at least 4 carbon atoms, which can be substituted, for example, by hydroxyl groups. In a particularly preferred embodiment, the deodorant or antiperspirant sticks according to the invention are free of soap gels or fatty acid salt gels, in particular, free of lithium, sodium, potassium, ammonium, diethanolamine and triethanolamine salts of fatty acids. Sticks on a soap base are incompatible with acidic antiperspirant active ingredients such as those that are used in the inventively preferred antiperspirant sticks. The solidification of the sticks according to the invention does not take place on the basis of inorganic and/or organic polymeric hydrogel formers, such as celluloses, cellulose derivatives, for example hydroxyalkylcelluloses, polyacrylates, veegum or bentones. In a particularly preferred embodiment, the sticks according to the invention are free of gels formed by inorganic and/or organic polymeric hydrogel formers. Besides the favorable active ingredient release, the formulation as oil-in-water dispersion/emulsion is accompanied by further advantages. First, the composition can be readily washed off of the skin. Second, during or following application to the skin, a therapeutic oil-in-water cream forms together with the skin moisture. In addition, the sticks according to the invention possess a pleasant and freshening skin feeling when applied on the skin. The sticks themselves have a very shiny, very white surface and a uniform, homogeneous structure. Compared to compositions from the prior art, the sticks according to the invention leave only minor white residues on the skin and on textiles. The sticks according to the invention show, in comparison to the prior art, particularly towards US 20060029624 A1 and U.S. 60/788,022 or PCT/EP2006/004371, an improved application behavior and easier slip on the skin as well as a lower adhesion. Surprisingly and unexpectedly to the person skilled in the art, it has been found that the oil components and the water-in-oil emulsifier or the water-in-oil emulsifier mixture have to be matched to one another with regard to their solubility parameters in order to form stick compositions with satisfactory performance-related hardnesses. Furthermore, in comparison with the prior art it was also surprisingly found that a lowering of the (average) HLB value of the oil-in-water emulsifier system enables a stable incorporation of more hydrophobic oil components that accordingly possess a lower Vaughan solubility parameter. This is all the more surprising as usually it is the case that to stabilize an oil-in-water emulsion, the emulsifier system must be all the more hydrophilic (i.e. the weight averaged HLB value must be all the more greater), the more hydrophobic the fatty phase is (i.e. the lower the Vaughan solubility parameter is). For the definition of the solubility parameter in the context of the present invention, reference is made to the publication, “Solubility—Effects in Product, Package, Penetration and Preservation,” by Chr. D. Vaughan in Cosmetics & Toiletries, vol. 103, October 1988, pages 47-69. The values for the solubility parameters published therein are noted in the non-SI unit (cal/cm³)^{0.5}. For the sake of simplicity, this non-SI unit will be retained in this specification. The values can be easily converted based on the relation 1 cal=4.1860 Joules.

Numerous solubility parameters tabulated by Vaughan in Cosmetics & Toiletries, Vol. 103, October 1988, pages 47-69, were calculated according to the Hildebrand equation (see C. D. Vaughan: J. Soc. Cosmet. Chem., Vol. 36, pp. 319-333 (September/October 1985) and the Hildebrand equation cited therein, and J. Am. Chem. Soc., vol. 38, pages 1442-1473 (1916) and J. Hildebrand and R. Scott: The Solubility of Nonelectrolytes, 3rd Edition, Reinhold Publ. Corp., New York, 1949); they are summarized below. Vaughan mentions that the solubility parameters can be calculated not only using the Hildebrand equation but also, for example, based on the evaporation enthalpy (Scatchard, J. Am. Chem. Soc., vol. 38, page 321 (1916)). All the calculation methods can produce different values for the solubility parameters, especially if the chemical material has an acid or base function. In the context of the present invention, it is preferred when the matching of the solubility parameters of the oil components and the water-in-oil emulsifier or the water-in-oil emulsifier mixture is performed only for solubility parameter values that were calculated using the same method. It is particularly preferred when the solubility parameter values that were calculated using the Hildebrand equation ((see C. D. Vaughan: J. Soc. Cosmet. Chem., vol. 36, pages 319-333 (September/October 1985)) are used for the matching according to the invention. If there is no available pair of solubility parameter values that were determined using the same method for a particular combination of oil component and water-in-oil emulsifier, it is also possible to use values that were determined using different methods, even experimental ones. However, that is a less preferred alternative according to the invention.

TABLE 1
Solubility parameter of various chemical components (from Cosmetics
& Toiletries, vol. 103, October 1988, pages 47-69)

| MATERIAL NAME (CTFA) | Solubility Parameter | |
|---|---|---|
| with Dielectric Constant | (cal/cm³)^{0.5} | Ref. |
| Helium (1.06) | 0.50 | *N |
| Hydrogen (1.23) | 2.50 | *N |
| Propellant 13 | 2.59 | *0 |
| Methane (1.70) | 4.70 | *0 |
| Neon | 4.90 | *N |
| Perfluorohexane | 5.68 | A |
| Perfluoroctane | 5.72 | A |
| Cyclomethicone D5 (2.50) | 5.77 | MO |
| Nitrogen (1.45) | 5.90 | *N |
| Dimethicone | 5.92 | *0 |
| Cyclomethicone D4 (2.39) | 5.99 | MO |
| Squalane | 6.03 | MO |
| Propellant 12 (2.13) | 6.11 | *0 |
| Hexamethyldisiloxane | 6.15 | MO |
| (2.17) | | |
| Isocetyl Stearate | 6.19 | M |
| Squalene | 6.19 | MO |
| Polytetrafluoroethylene | 6.20 | * |
| Propane | 6.21 | *0 |
| Propellant 22(6.11) | 6.23 | MO |
| Perfluorodecalin | 6.34 | A |
| Neopentane | 6.38 | CO |
| Safflower Oil | 6.42 | L1 |
| Melene (C30) | 6.58 | C |
| Docosane (C22) | 6.60 | I |
| Almond Oil | 6.81 | L1 |
| Isopentane | 6.82 | CO |
| Avocado Oil | 6.83 | L1 |
| Nonacosane (D29) | 6.83 | C |
| Arachidic Acid | 6.85 | H |
| Pristane | 6.85 | MO |
| Decyl Oleate | 6.92 | M |
| C8-Isoparaffin (1.94) | 6.93 | MO |
| Diisopropyl Ether (3.88) | 6.95 | KE |
| Argon (1.53) | 7.00 | *N |
| Sperm Oil | 7.09 | *0 |
| White Mineral Oil | 7.09 | *0 |
| Pentane | 7.10 | *0 |
| Tricosane (C23) | 7.13 | C |
| Isodecyl Oleate | 7.17 | M |
| Propellant 113 | 7.19 | H |
| Oxygen (1.50) | 7.20 | *N |
| Cholesteryl Oleate | 7.24 | * |
| Peanut Oil | 7.74 | L1 |
| Hexane (1.88) | 7.28 | CO |
| Linseed Oil | 7.29 | *0 |
| Octadecane (C18) | 7.29 | C |
| Isopropyl Myristate | 8.02 | 0 |
| Turpentine (pinene)(2.70) | 8.03 | CO |
| Human Erythrocyte | 8.05 | * |
| Methyl Oleate (3.21) | 8.05 | CO |
| Cetyl Acetate | 8.06 | 0 |
| Methyl Linoleate | 8.08 | C |
| Isostearic Acid | 8.09 | 0 |
| Coconut Oil | 8.10 | L1 |
| Myristic Acid (C14) | 8.10 | I0 |
| Dibutylamine | 8.15 | * |
| Eucalyptol (Cineole) | 8.17 | L1 |
| Natural Rubber | 8.20 | H |
| Octylamine | 8.21 | A |
| Propylene Glycol | 8.21 | L1 |
| Dipelargonate | | |
| Titanium Isopropoxide | 8.21 | M |
| Melissyl Alcohol | 8.22 | CO |
| (C30) | | |
| Glycol Distearate | 8.24 | J3 |
| Glycol Stearate | 8.28 | J3 |
| Capric/Caprylic Triglycerid | 8.29 | L1 |
| Isosteareth-2 | 8.29 | L1 |
| PPG-2 Myristyl Ether | 8.29 | L1 |
| Ricinoleic Acid | 8.30 | C |
| Staphylococcus Aureus | 8.30 | P |
| Glyceryl Isostearate | 8.31 | J3 |
| Glyceryl Stearate (mono) | 8.31 | *0 |
| Laureth-4 | 8.31 | J3 |
| Limonene (2.30) | 8.33 | C |
| Propylene Glycol Laurate | 8.33 | L1 |
| Octyl Mercaptan | 8.35 | K |
| PEG-2 Stearate | 8.36 | J3 |
| Ethyl Caprate (C10) | 8.39 | A |
| Radon | 8.40 | *N |
| Amyl Acetate | 8.43 | C |
| Glyceryl Stearate SE | 8.43 | J3 |
| Diisopropyl Adipate | 8.46 | E0 |
| Lauric Acid (C12) | 8.46 | I0 |
| Polyethylene (2.35) | 8.50 | *0 |
| Diisopropyl Amine | 8.51 | *0 |
| Polyglyceryl-3 Oleate | 8.52 | J3 |
| Ethylene/Vinyl Acetate | 8.55 | *0 |
| (AC400) | | |
| Ethyl Caprylate (C8) | 8.57 | A |
| Octyl Acetate | 8.58 | A |
| Octyl Iodide | 8.58 | A |
| Ethyl Oleate (3.17) | 8.60 | * |
| Isopropylbenzene (12.38) | 8.60 | * |
| Sorbitan Laurate | 8.61 | 0 |
| Behenyl Alcohol (C22) | 8.63 | I0 |
| Carbon Tetrachloride | 8.64 | C |
| (2.23) | | |
| Butyl Mercaptan | 8.65 | KA |
| Isostearyl Alcohol | 8.67 | 0 |
| Lauraldehyde | 8.68 | A |
| Ethyl Caproate (C6) | 8.69 | A |
| Cholesteryl Propionate | 8.70 | * |
| Isocetyl Alcohol | 8.71 | M |
| Bornyl Acetate | 8.74 | CA |
| Ethyl Mercaptan | 8.75 | K |
| Decanone-2 | 8.76 | A |
| Octanal | 8.77 | C |
| Trifluoroactylacetone | 8.77 | A |
| Cholesteryl Myristate | 8.80 | * |
| Zinc Stearate | 8.80 | 0 |
| Citronella | 8.83 | CO |
| Diethyl Ketone (17.00) | 8.85 | E |
| Methyl Isobutyl Ketone | 8.85 | E0 |
| (14.70) | | |
| Oxidized Polyethylene | 8.85 | *0 |
| (AC629) | | |
| Methyl Heptyl Ketone | 8.86 | A |
| Myristyl Lactate | 8.87 | M |
| Capric Acid (C10) | 8.88 | I0 |
| Methyl Caproate (CB) | 8.88 | B |
| Arachidyl Alcohol (C20) | 8.89 | CO |
| Dipropyl Ketone | 8.89 | C |
| Muscone | 8.89 | CO |
| Candida Albicans | 8.90 | P |
| Castor Oil | 8.90 | H |
| Elaidyl Alcohol | 8.90 | CO |
| beta-Ionone | 8.90 | CO |
| Polystyrene | 8.90 | M |
| Nicoteine | 10.08 | C |
| Octanol/Caprylic (C8) | 10.09 | CO |
| Alcohol (10.34) | | |
| Acetic Anhydride (22.40) | 10.12 | C |
| Nerol | 10.13 | C |
| Ethyl Cinnamate | 10.14 | A |
| Diethyl Nitrosamine | 10.16 | C |
| Octyl Salicylate | 10.17 | M |
| Griseofulvin | 10.20 | M |
| Dioctyl Malate | 10.21 | M |
| Geraniol | 10.21 | CO |
| Butyl Lactate | 10.27 | AO |
| t-Butyl Alcohol (10.90) | 10.28 | CO |
| Morpholine (7.33) | 10.28 | C |
| Homosalate | 10.29 | GM |
| Valeric Acid (C5) | 10.29 | A |
| Polyethylene | 10.30 | * |
| Terephthalate (PET) | | |
| Pyridine (12.3) | 10.30 | A |
| Phenyl Acetate (5.23) | 10.33 | E |
| Thiolacetic Acid | 10.38 | A |
| Methoxypropanol | 10.40 | * |
| Diethyl Toluamide | 10.46 | M |
| Nonoxynol-1 | 10.47 | * |
| Borneol | 10.48 | C |
| Methyl Benzoate (6.59) | 10.48 | E |
| Hexyl Alcohol (13.30) | 10.50 | I0 |
| SAN (85/15) | 10.50 | * |
| Butoxyethanol (9.30) | 10.53 | E |
| Formaldehyde | 10.54 | C |
| o-Nitrotoluene (27.40) | 10.55 | B |
| Butylparaben | 10.57 | * |
| Propionitrile | 10.57 | A |
| Tripropylene Glycol (PPG-3) | 10.60 | M |
| Methyl Salicylate (9.41) | 10.62 | C0 |
| Acetophenone (17.39) | 10.64 | C |
| Diacetone Alcohol (18.20) | 10.67 | CO |
| Ethyl Anthranilate | 10.67 | C |
| Naphthylene | 10.74 | B |
| Phenylpentanol | 10.74 | A |
| Butyric Acid (2.97) | 10.76 | E |
| Cyclopentanone | 10.77 | E |
| Thymol | 10.77 | C |
| Triacetin | 10.77 | 0 |
| Methoxyethanol(16.90) | 10.80 | * |
| Amyl Alcohol (13.90) | 10.84 | CE |
| Ethanedithiol | 10.87 | A |
| Ethyl Hexanediol | 10.89 | A |
| Trichloroacetic Acid | 10.89 | E |
| Benzalphthalide | 10.90 | 0* |
| Testosterone | 10.90 | * |
| Cinnamaldehyde | 10.92 | C |
| Propylparaben | 10.94 | GM |
| Valine | 10.94 | J1 |
| Tolbutamide | 10.98 | * |
| Benzaldehyde(17.80) | 11.00 | CO |
| Triisopropanolamine | 11.02 | M |
| Phenylbutanol | 11.04 | A |
| Eugenol | 11.12 | C |
| D&C Red 22 (Eosin) | 11.15 | L2 |
| Butyl Alcohol (17.51) | 11.18 | C0 |
| Cellulose Acetate | 11.20 | H |
| Methyl Anthranilate | 11.22 | C |
| Caproamide (C6) | 11.24 | M |
| Isopropyl Alcohol (18.30) | 11.24 | C0 |
| Nitrocellulose | 11.25 | M0 |
| Hexobarbital | 11.30 | J1 |
| Secobarbital | 11.30 | J1 |
| p-Anisaldehyde | 11.32 | A |
| PEG-8 | 11.34 | MO |
| Panthenol | 11.39 | MO |
| Propionic Acid (3.35) | 11.40 | EA |
| Glyoxal | 11.46 | C |
| Phenylpropanol | 11.46 | A |
| Methyl Lactate | 11.47 | CO |
| PEG-6 (16.00) | 11.47 | D0 |
| PEG-5 (18.16) | 11.54 | D0 |
| Phenylalanine | 11.57 | G |
| Propylene Glycol (32.00) | 14.00 | CO |
| Theophyllin | 14.00 | * |
| Aspartic Acid | 14.11 | J1 |
| Pyrrolidinone-2 | 14.22 | |
| Ethylene Glycol(37.00) | 14.50 | CO |
| Hydroquinone | 14.62 | |
| Lactic Acid (22.00) | 14.81 | |
| Resorcinol | 14.96 | C |
| Histidine | 15.25 | J1 |
| Ethanolamine (37.72) | 15.41 | *M |
| Sodium Capryl Sulfate | 15.80 | * |
| (14.84) | | |
| Erythritol | 16.06 | * |
| Formamide (109.0) | 17.82 | E |
| Lactose | 19.50 | * |
| Cyclohexane (2.02) | 7.30 | E |
| Dioctyl Ether | 7.30 | A |
| Eicosane (020) | 7.32 | C |
| Lanolin Oil | 7.33 | L1 |
| Petrolatum | 7.33 | *0 |
| Behenic Acid | 7.35 | I0 |
| Diethyl Ether (4.34) | 7.37 | CO |
| Corn Oil-Refined | 7.40 | L1 |
| Cetane (016) | 7.41 | I |
| Heptane (1.92) | 7.41 | CO |
| Isostearyl | 7.43 | M |
| Neopentanoate | | |
| Octyl Palmitate | 7.44 | 0 |
| Propyl Fluoride | 7.48 | C |
| Rice Oil —SO | 7.48 | L1 |
| Tridecane (C13) | 7.48 | CO |
| Propellant 11 | 7.49 | 0 |
| (2.28) | | |
| Cottonseed Oil | 7.52 | L1 |
| Carbon Dioxide | 7.53 | H |
| (1.60) | | |
| Isopropyl Linoleate | 7.55 | M |
| Cod Liver Oil | 7.56 | L1 |
| Erucic Acid | 7.57 | CO |
| Octane (1.95) | 7.58 | MO |
| Cetyl Octanoate | 7.59 | M |
| Decene-1 | 7.59 | C |
| Dodecene (2.01) | 7.59 | C |
| (7.65-I) | | |
| Diethylhexyl Adipate | 7.60 | M |
| Decane (1.99) | 7.62 | CO |
| C12-15 Alcohols | 7.63 | MO |
| Benzoate | | |
| Isobutyl Stearate | 7.65 | 0 |
| Butyl Myristate | 7.68 | D |
| Butyl Stearate(3.11) | 7.68 | CO |
| Stearic Acid | 7.74 | IO |
| (C18)(2.30) | | |
| Dioctyl Maleate | 7.75 | 0 |
| Octyl Fluoride | 7.76 | AG |
| Isopropyl Palmitate | 7.78 | 0 |
| Dioctyl Adipate | 7.82 | M |
| Oleth-3 | 7.83 | *0 |
| Diethyl Amine | 7.86 | C |
| Linolenic Acid | 7.86 | C0 |
| Olive Oil | 7.87 | *0 |
| Palmitic Acid (C16) | 7.89 | IO |
| (22.30) | | |
| Oleic Acid(2.46) | 7.91 | IO |
| PEG-4 Stearate | 7.92 | 0 |
| Tetraethyl Lead | 7.92 | E |
| Tridecyl | 7.92 | L1 |
| Neopentanoate | | |
| Pentaerythrityl | 7.98 | L1 |
| Tetraoleate | | |
| Tocopheryl Acetate | 7.98 | M |
| Ethyl Myristate | 8.00 | C |
| Stearyl Alcohol (C18) | 8.90 | I0 |
| Methyl Hexyl Ketone | 8.91 | A |
| Octyl Dodecanol | 8.92 | OM |
| Butyl Acetate (5.01) | 8.93 | CO |
| Cetyl Alcohol (CIG) | 8.94 | I0 |
| alpha-Thujone | 8.94 | A |
| Toluene (2.38) | 8.94 | C |
| Oleyl Alcohol | 8.95 | CO |
| Propylene Oxide | 8.99 | A |
| Aspergillus Niger | 9.00 | P |
| Octyl Dimethyl PABA | 9.01 | OM |
| 9.34 G | | |
| Propyl Acetate | 9.02 | CO |
| Chloroform | 9.05 | A |
| Benzene (2.28) | 9.08 | E |
| PEG-20 Stearate | 9.08 | J3 |
| Ceteth-20 | 9.10 | H |
| Methyl Butyl | 8.10 | M |
| Methacrylate CO | | |
| Octyl | 9.10 | M |
| Methoxycinnamate | | |
| Methyl Butyl Ketone | 9.11 | E |
| Myristyl Alcohol | 9.16 | IO |
| (C14) | | |
| Polysorbate-20 | 9.16 | J3 |
| THF (7.58) | 9.16 | E |
| BHT | 9.17 | D |
| Tocopherol | 9.17 | M |
| Lauryl Lactate | 9.18 | M |
| PEG-40 Stearate | 9.18 | J3 |
| Ethyl Acetate (6.02) | 9.19 | CO |
| Tributyl Citrate | 9.20 | M |
| Ethyl Acrylate | 9.22 | A |
| Propionaldehyde | 9.22 | A |
| Methyl Propyl Ketone | 9.27 | C |
| Dipropyl Nitrosamine | 9.29 | B |
| alpha-Bisabolol | 9.30 | M |
| Pseudomonas Aeroginosa | 9.30 | P |
| Trichomonas Ment. | 9.30 | P |
| Caprylic Acid | 9.32 | E0 |
| (C8)(2.45) | | |
| Cetyl Lactate | 9.32 | M |
| PEG-100 Stearate | 9.35 | J3 |
| Trimethyl Citrate | 9.39 | H |
| Klebsiella Pneumoniae | 9.40 | P |
| Methyl Methacrylate | 9.40 | H |
| Copolymer | | |
| Nicotine | 9.40 | C |
| Camphor | 9.45 | C |
| Oxidized Polyethylene | 9.50 | *0 |
| (AC392) | | |
| Lauryl Alcohol (C12) | 9.51 | C0 |
| Pulegone | 9.51 | A |
| Cholesterol | 9.55 | 0 |
| Ethylene/Vinyl | 9.55 | *0 |
| Acetate (AC430) | | |
| Methylene Chloride | 9.55 | E |
| (9.08) | | |
| Dimethyl Isosorbide | 9.58 | M |
| PPG-2 Methyl Ether | 9.60 | * |
| Acetaldehyde (21.8) | 9.61 | A |
| Undecyl Alcohol | 9.51 | C0 |
| Linalool | 9.62 | C |
| Methyl Ethyl Ketone | 9.63 | C0 |
| (18.50) 9.53A | | |
| Acetylacetone | 9.68 | * |
| Amyl Dimethyl PABA | 9.72 | M |
| Methyl Iodide | 9.75 | C |
| Decyl Alcohol (C10) | 9.78 | C0 |
| (8.10) | | |
| Chlorine | 9.80 | *H |
| Ethylhexanol | 9.80 | A |
| Stratum Corneum- | 9.80 | * |
| Porcine | | |
| Acetone (20.70) | 9.87 | C |
| Citronellol | 9.88 | A |
| Dibutyl Phthalate | 9.88 | M |
| (6.44) | | |
| Menthyl Anthranilate | 9.89 | M |
| PPG-4 | 9.89 | M |
| Ethoxyethanol | 9.90 | *M |
| (29.60) | | |
| Ethylene Oxide | 9.93 | A |
| (13.90) | | |
| Menthol | 9.94 | C0 |
| Tributyrin | 9.97 | 0 |
| Butoxydiglycol- | 9.98 | * |
| BuCarbitol | | |
| Nitrous Oxide | 10.00 | *H |
| (1.60) | | |
| Dioxane (2.21) | 10.01 | * |
| Ethyl Benzoate | 10.01 | C |
| (6.02) | | |
| Caproic Acid (C8) | 10.05 | E0 |
| (2.53) | | |
| Salicylic Acid | 10.06 | C |
| Copper | 11.60 | * |
| Acetylacetonide | | |
| Sulfamethoxazole | 11.60 | J1 |
| PEG-4 (20.44) | 11.61 | DO |
| Acetohexamide | 11.64 | * |
| N-Methylpyrrolidone | 11.71 | A |
| Propyl Alcohol | 11.73 | CO |
| (20.10) | | |
| Dimethyl Nitrosamine | 11.74 | C |
| Pentobarbital | 11.75 | J1 |
| Butadiene Diepoxide | 11.78 | A |
| Dipropylene Glycol | 11.78 | M |
| (PPG-2) | | |
| Phthalide | 11.78 | C |
| Lysine | 11.79 | J1 |
| Phenethyl Alcohol | 11.79 | CO |
| Acetonitrile | 11.81 | AO |
| (37.5) | | |
| Cinnamic Acid | 11.83 | C |
| p-Nitrotoluene | 11.83 | |
| (24.20) | | |
| Phenoxyethanol | 11.87 | CO |
| Butobarbital | 11.90 | J1 |
| Sulfadiazine | 11.90 | * |
| Butalbital | 11.95 | J1 |
| Cinnamyl Alcohol | 11.96 | C |
| Sorbic Acid | 11.97 | MO |
| Methylparaben | 11.98 | 0 |
| Hydroxyanisole | 12.00 | C |
| Benzocaine | 12.05 | * |
| Triethylene Glycol | 12.21 | MO |
| (23.69) | | |
| Alanine | 12.23 | J1 |
| Nitromethane | 12.27 | C |
| Benzyl Alcohol(I3.10) | 12.31 | 0 |
| Hexylene Glycol | 12.32 | * |
| Butyramide | 12.33 | A |
| Human Serum | 12.33 | J1 |
| Albumin A | | |
| Vanillin | 12.34 | D |
| BHA | 12.37 | 0 |
| Acetic Acid (6.15) | 12.40 | CO |
| Cyclobarbital | 12.40 | J1 |
| Diisopropanolamine | 12.40 | A |
| Ethyl | 12.42 | M |
| Dihydroxypropyl | | |
| PABA | | |
| o-Propylene Diamine | 12.43 | D |
| p-Dinitrobenzene | 12.49 | B |
| Ethyl Alcohol | 12.55 | CO |
| (24.30) | | |
| Rat Gut Membrane | 12.60 | * |
| Sulfamethazine | 12.60 | J1 |
| Sulfisomidinc | 12.70 | J1 |
| Sulfur (3.55) | 12.70 | *N |
| Phenol (9.78) | 12.79 | CE |
| Sulfisomidine | 12.80 | * |
| Allobarbital | 12.85 | J1 |
| o-Nitroaniline (34.50) | 12.88 | D |
| Pyruvic Acid | 12.94 | * |
| Phenobarbital | 13.00 | J1 |
| Isopropanolamine | 13.02 | A |
| Adipic Acid | 13.04 | 0 |
| BAL (2,3- | 13.10 | B |
| Dimercapto-1- | | |
| propanol) | | |
| Sulfathiazole | 13.10 | * |
| Aminoethyl | 13.18 | M |
| Ethanolamine | | |
| Glutathione | 13.18 | G |
| Butylene Glycol | 13.20 | CO |
| m-Nitroaniline | 13.23 | C |
| Triethanolamine | 13.28 | MO |
| (29.36) | | |
| Propylene Carbonate | 13.35 | * |
| (65.00) | | |
| Benzamide | 13.38 | B |
| Dimethyl Sulfoxide | 13.40 | H |
| (46.68) | | |
| Sulfamerazine | 13.40 | 31 |
| Propionamide | 13.46 | AC |
| Barbital | 13.50 | J1 |
| Mercaptoethanol | 13.55 | A |
| Propiolactone | 13.56 | A |
| Diethylene Glycol | 13.61 | E0 |
| (31.70) | | |
| Propargyl Alcohol | 13.61 | A |
| p-Nitroaniline (56.30) | 13.67 | A |
| Caffeine | 13.80 | * |
| Thiodiglycol | 13.80 | M |
| Thioglycolic Acid | 13.86 | A |
| Sulfameter | 13.90 | J1 |
| Diethanolamine | 13.95 | M |
| Pyrrolidone | 14.00 | * |
| Hexyl Resorcinol | 14.06 | * |
| Sodium Lauryl | 14.18 | * |
| Sulfate | | |
| Methyl Alcohol | 14.33 | CO |
| (32.70) | | |
| Urea | 14.50 | G |
| Formic Acid (58.5) | 14.72 | E |
| PABA 14.56 G | 14.82 | DO |
| Acetamide MEA | 15.11 | M |
| p-Hydroxybenzoic | 15.30 | * |
| Acid | | |
| Pyrogallol | 15.41 | A |
| Acetamide (59.00) | 16.03 | C |
| Glycerin (42.50) | 16.26 | E0 |
| Ammonia (16.90) | 18.08 | 0 |
| Water (80.10) | 23.40 | CN |

References
NOTE:
*= Solubility Parameter value from literature

### SOURCE OF PHYSICAL DATA

A. Aldrich Chemical Co, Catalog 1986 gram
B. Beilstein's Index
C. Chemical Rubber Handbook of Chemi. & Physics, 42d Ed. (1961-1962)
D. Dictionary of Organic Compounds
E. Eastman Organic Chemical Bulletin 47, No. 1, 1975
F. Fisher Scientific Catalog—1986
G. Group Contribution Method of Hay, Van Krevelen and Feodors.
H. HANDBOOK OF SOLUBILITY PARAMETERS, A. F. Barton, Chemical Rubber Pub 1.1985
I. INDUSTRIAL WAXES, H. Bennett, Chemical Pub. Co.
J. Journal Reference by number 0(x).
J1 J. Pharm. Sci. 75, (7), 639
J2 Pharm. Acta Helv., 48, 549 (1973)
J3 Am. Cosmet. Perf., 87, p. 85 (1972)
K. Kolthof & Elving: TREATISE on ANALYTICAL CHEMISTRY
L. Laboratory Determination by:
L(1) Consolbilizer Study
L(2) Solubility Study Unpublished
M. Manufacturer's Physical Date by Personal Communication
N. Hildebrand & Scott: The Solubility of Nonelectrolytes. Dover Press
O. Original published values JSCC 36, 319
P. Pharm. Acta Helv. 81, (3), 95 Antimicrobial Activity and Solubility Parameters-C.V./F.W.

In the stick compositions according to the invention, the (average) solubility parameter of the totality of the oils present deviates by at most −1.0 (cal/cm³)^{0.5} or at most +1.0 (cal/cm³)^{0.5}, preferably by at most −0.8 (cal/cm³)^{0.5} or at most +0.8 (cal/cm³)^{0.5}, particularly preferably by at most −0.7 (cal/cm³)^{0.5} or at most +0.7 (cal/cm³)^{0.5}, extremely preferably by at most −0.6 (cal/cm³)^{0.5} or at most +0.4 (cal/cm³)^{0.5} from the (average) solubility parameter of the water-in-oil emulsifier/water-in-oil emulsifiers.

If water-in-oil emulsifier mixtures or oil mixtures are used, the average solubility parameter of the mixture is considered in each case, specifically the arithmetic mean according to the weight fractions of the individual components. In the context of the invention, it is also possible for a weight fraction of up to 20 wt. % of the oils that are liquid under normal conditions to consist of oils whose solubility parameter deviates by more than −1.0 (cal/cm³)^{0.5} or by more than +1.0 (cal/cm³)^{0.5} from the (average) solubility parameter of the water-in-oil emulsifier (mixture). In a particularly preferred embodiment of the invention, no oils that are in the liquid state under normal conditions are present whose solubility parameter deviates by more than ±1.2 (cal/cm³)^{0.5} from the (average) solubility parameter of the water-in-oil emulsifier/water-in-oil emulsifiers.

Wax Matrix

The wax matrix of the stick compositions according to the invention comprises at least one wax component with a melting point of >50° C., which is not included in the nonionic oil-in-water emulsifiers with an HLB value of more than 7 or in the nonionic water-in-oil emulsifiers with an HLB value greater than 1.0 and less than or equal to 7.0.

Generally, waxes are of solid to brittle consistency, coarse to finely crystalline, transparent to opaque, but not vitreous, and melt above 50° C. without decomposition. Just a little above the melting point they are of low viscosity and exhibit a strongly temperature-dependent consistency and solubility.

According to the invention, preference is given, for example, to natural vegetal waxes, e.g., candelilla wax, carnauba wax, Japan wax, sugar cane wax, ouricoury wax, cork wax, sunflower wax, fruit waxes, such as orange waxes, lemon waxes, grapefruit wax, and animal waxes, e.g., beeswax, shellac wax and spermaceti. In the context of the present invention, it may be particularly preferred to use hydrogenated or hardened waxes. Chemically modified waxes, in particular the hard waxes, such as, for example, montan ester waxes, hydrogenated jojoba waxes and Sasol waxes, can also be used as the wax component. Synthetic waxes, which are likewise preferred according to the invention, include, for example, polyalkylene waxes and polyethylene glycol waxes, C₂₀-C₄₀ dialkyl esters of dimer acids, C₃₀-C₅₀ alkyl beeswax and alkyl and alkylaryl esters of dimer fatty acids.

A particularly preferred wax component is chosen from among at least one ester of a saturated monohydric C₁₆-C₆₀ alcohol and a saturated C₈-C₃₆ monocarboxylic acid. According to the invention these also include lactides, the cyclic double esters of α-hydroxycarboxylic acids of the corresponding chain length. Esters of fatty acids and long-chain alcohols have proven particularly advantageous for the composition according to the invention because they impart excellent sensory properties to the stick according to the invention, particularly to the antiperspirant preparation according to the invention, and high stability to the stick overall. The esters are composed of saturated, branched or unbranched monocarboxylic acids and saturated, branched or unbranched monohydric alcohols. According to the invention, it is also possible to use esters of aromatic carboxylic acids or hydroxycarboxylic acids (e.g. 12-hydroxystearic acid) and saturated, branched or unbranched alcohols in so far as the wax component has a melting point of >50° C. It is particularly preferred to choose the wax components from the group of esters of saturated, branched or unbranched alkanecarboxylic acids with a chain length of 12 to 24 carbon atoms and the saturated, branched or unbranched alcohols with a chain length of 12 to 50 carbon atoms which have a melting point of >50° C. In particular, C₁₆₋₃₆ alkyl stearates and C₁₈₋₃₈ alkyl hydroxystearoylstearates, C₂₀₋₄₀ alkyl erucates and cetearyl behenate may be advantageous as the wax component. The wax or the wax components have a melting point of >50° C., preferably >60° C. A particularly preferred embodiment of the invention comprises a C₂₀-C₄₀ alkyl stearate as the wax component. This ester is known under the name Kesterwachs® K82H or Kesterwachs® K80H and is sold by Koster Keunen, Inc. It is the synthetic, imitation of the monoester fraction of beeswax and is characterized by its hardness, its oil gelability and its broad compatibility with lipid components. This wax can be used as a stabilizer and as a consistency regulator for W/O and O/W emulsions. Kesterwachs offers the advantage that, even in low concentrations, it has excellent oil gelability and thus does not make the stick compound too heavy and allows for a velvety release. A further particularly preferred embodiment of the invention comprises cetearyl behenate, i.e. mixtures of cetyl behenate and stearyl behenate, as the wax component. This ester is known under the name Kesterwachs® K62 and is sold by Koster Keunen, Inc.

Further preferred lipid or wax components with a melting point of >50° C. are the triglycerides of saturated and optionally hydroxylated C₁₂₋₃₀ fatty acids, such as hardened triglyceride fats (hydrogenated palm oil, hydrogenated coconut oil, hydrogenated castor oil), glyceryl tribehenate (tribehenin) or glyceryl tri-12-hydroxystearate, also synthetic fully esterified fatty acids and glycols or polyols having 2-6 carbon atoms as long as they have a melting point above 50° C., for example, preferably C₁₈-C₃₆ acid triglyceride (Syncrowax® HGL-C). According to the invention, hydrogenated castor oil, obtainable, e.g., as the commercial product Cutina® HR, is particularly preferred as the wax component. Further preferred wax components with a melting point of >50° C. are the saturated linear C₁₄-C₃₆ carboxylic acids, in particular myristic acid, palmitic acid, stearic acid and behenic acid, and mixtures of these compounds, e.g., Syncrowax® AW 1C (C₁₈-C₃₆ fatty acids) or Cutina® FS 45 (mixture of palmitic and stearic acid).

Preferred sticks according to the invention, particularly deodorant or antiperspirant sticks, are characterized in that the wax component a) is chosen from among esters of a saturated, monohydric C₁₆-C₆₀ alkanol and a saturated C₈-C₃₆ monocarboxylic acid, in particular lauryl laurate, lauryl myristatee, lauryl palmitate, lauryl stearate, lauryl 12-hydroxystearate, lauryl eicosanate, lauryl behenate, lauryl lignocerate, lauryl cerate, lauryl myricate, myristyl laurate, myristyl myristate, myristyl palmitate, myristyl stearate, myristyl 12-hydroxystearate, myristyl eicosanate, myristyl behenate, myristyl lignocerate, myristyl cerate, myristyl myricate, cetyl laurate, cetyl myristate, cetyl palmitate, cetyl stearate, cetyl 12-hydroxystearate, cetyl eicosanate, cetyl behenate, cetyl lignocerate, cetyl cerate, cetyl myricate, stearyl laurate, stearyl myristate, stearyl palmitate, stearyl stearate, stearyl 12-hydroxystearate, stearyl eicosanate, stearyl behenate, stearyl lignocerate, stearyl cerate, stearyl myricate, 12-hydroxystearyl laurate, 12-hydroxystearyl myristate, 12-hydroxystearyl palmitate, 12-hydroxystearyl stearate, 12-hydroxystearyl 12-hydroxystearate, 12-hydroxystearyl eicosanate, 12-hydroxystearyl behenate, 12-hydroxystearyl lignocerate, 12-hydroxystearyl cerate, 12-hydroxystearyl myricate, arachyl laurate, arachyl myristate, arachyl palmitate, arachyl stearate, arachyl 12-hydroxystearate, arachyl eicosanate, arachyl behenate, arachyl lignocerate, arachyl cerate, arachyl myricate, behenyl laurate, behenyl myristate, behenyl palmitate, behenyl stearate, behenyl 12-hydroxystearate, behenyl eicosanate, behenyl behenate, behenyl lignocerate, behenyl cerate, behenyl myricate, lignoceryl laurate, lignoceryl myristate, lignoceryl palmitate, lignoceryl stearate, lignoceryl 12-hydroxystearate, lignoceryl eicosanate, lignoceryl behenate, lignoceryl lignocerate, lignoceryl cerate, lignoceryl myricate, ceryl laurate, ceryl myristate, ceryl palmitate, ceryl stearate, ceryl 12-hydroxystearate, ceryl eicosanate, ceryl behenate, ceryl lignocerate, ceryl cerate, ceryl myricate, myricyl laurate, myricyl myristate, myricyl palmitate, myricyl stearate, myricyl 12-hydroxystearate, myricyl eicosanate, myricyl behenate, myricyl lignocerate, myricyl cerate, myricyl myricate, particularly preferably cetyl behenate, stearyl behenate and C₂₀-C₄₀ alkyl stearates, in particular arachyl stearate, behenyl stearate, lignoceryl stearate, ceryl stearate and myricyl stearate, also selected from glycerine triesters of saturated linear C₁₂-C₃₀ carboxylic acids that can be hydroxylated, wherein these glycerine triesters are preferably in the form of natural waxes, in particular candelilla wax, carnauba wax or beeswax, or preferably in the form of natural oils that are fully hydrogenated (hardened), in particular fully hydrogenated hardened castor oil (tri-12-hydroxystearine), tristearine, tribehenine, fully hydrogenated soya bean oil, fully hydrogenated maize germ oil, fully hydrogenated sunflower oil, fully hydrogenated erucic acid enriched rape seed oil (HEAR oil), fully hydrogenated erucic acid-poor rape seed oil (LEAR oil), fully hydrogenated canola oil, fully hydrogenated crambe oil, fully hydrogenated meadow foam seed oil, fully hydrogenated cotton seed oil, fully hydrogenated olive oil, fully hydrogenated thistle oil, fully hydrogenated sunflower oil, fully hydrogenated sesame oil, fully hydrogenated coco oil, fully hydrogenated palm oil, fully hydrogenated palm seed oil, fully hydrogenated babassu oil, fully hydrogenated peanut oil, fully hydrogenated cocoa butter, shea butter, illipe butter, hardened animal fats, in particular tallow or rind, fully hydrogenated oils of marine origin, such as swordfish oil, sardine oil, whale oil and herring oil.

Further preferred sticks according to the invention, in particular deodorant or antiperspirant sticks, are characterized in that the wax component a) is selected from saturated linear C₁₄-C₃₆ carboxylic acids, in particular myristic acid, palmitic acid, stearic acid, 12-hydroxystearic acid, eicosanoic acid, behenic acid, lignoceric acid, ceric acid, myricic acid, as well as mixtures thereof. Particularly preferred mixtures of wax components a) are selected from mixtures of cetyl behenate, stearyl behenate, hardened castor oil, palmitic acid and stearic acid. Further particularly preferred mixtures of wax component a) are selected from mixtures comprising C₂₀-C₄₀ alkyl stearate, hardened castor oil, palmitic acid and stearic acid. Further particularly preferred mixtures of wax component a) are selected from mixtures comprising C₁₆-C₂₀ alkyl behenate, hardened castor oil, palmitic acid and stearic acid. Further particularly preferred mixtures of wax component a) are selected from mixtures comprising arachyl stearate, behenyl stearate, lignoceryl stearate, ceryl stearate, myricyl stearate, hardened castor oil, palmitic acid and stearic acid. Further particularly preferred mixtures of wax component a) are selected from mixtures comprising palmityl behenate, stearyl behenate, arachyl behenate, hardened castor oil, palmitic acid and stearic acid.

Further preferred sticks according to the invention, in particular deodorant or antiperspirant sticks, are characterized in that the total amount of wax component(s) a) is 4-20 wt. %, preferably 7-15 wt. %, exceptionally preferably 10 to 12 wt. % relative to the overall composition. In a particularly preferred embodiment, the ester/esters of a saturated, monohydric C₁₂-C₆₀ alcohol and a saturated C₈-C₃₆ monocarboxylic acid, which represent(s) the wax component(s) a), comprise(s) 2-10 wt. %, preferably 2-6 wt. %, most preferably 3-5 wt. %, relative to the overall composition.

Oil-in-Water Emulsifiers

The stick compositions according to the invention comprise at least one nonionic oil-in-water emulsifier with an HLB value of more than 7, wherein in fact the total oil-in-water emulsifier system has a weight average HLB value in the range 11-16. These are emulsifiers generally known to the person skilled in the art, as listed, for example, in Kirk-Othmer, “Encyclopedia of Chemical Technology,” 3rd edition, 1979, volume 8, pages 913-916. For ethoxylated products, the HLB value is calculated according to the formula HLB=(100−L):5, where L is the weight fraction of the lipophilic groups, i.e. of the fatty alkyl or fatty acyl groups, in the ethylene oxide adducts, expressed in percent by weight. In selecting nonionic oil-in-water emulsifiers that are suitable according to the invention, it is particularly preferred to use a mixture of nonionic oil-in-water emulsifiers in order to be able to optimally adjust the stability of the stick compositions according to the invention. Here, the individual emulsifier components contribute to the overall HLB value or average HLB value of the oil-in-water emulsifier mixture according to their quantitative proportion of the total amount of the oil-in-water emulsifiers. According to the invention, the average HLB value of the oil-in-water emulsifier mixture is 11-17, preferably 12-15 and particularly preferably 13-14. In order to achieve such average HLB values, oil-in-water emulsifiers from the HLB value ranges 10-14, 14-16 and optionally 15-17 are preferably combined with one another. The oil-in-water emulsifier mixtures can, of course, also comprise nonionic emulsifiers with HLB values in the range from >7-10 and 17-20; such emulsifier mixtures may likewise be preferred according to the invention. However, in another preferred embodiment, the sticks according to the invention, in particular the deodorant or antiperspirant sticks can also comprise just one oil-in-water emulsifier with an HLB value in the range of 11-17, preferably 12-15 and particularly preferably 13-14. Preferred sticks according to the invention, in particular the deodorant or antiperspirant sticks are characterized in that the nonionic oil-in-water emulsifiers b) are chosen from among ethoxylated C₈-C₂₄ alkanols with, on average, 5-100 mol ethylene oxide per mole, ethoxylated C₈-C₂₄-carboxylic acids with, on average, 5-100 mol ethylene oxide per mole, silicone copolyols with ethylene oxide units or with ethylene oxide and propylene oxide units, alkyl mono- and oligoglycosides having 8 to 22 carbon atoms in the alkyl moiety, and ethoxylated analogs thereof, ethoxylated sterols, partial esters of polyglycerols with 2 to 10 glycerol units and esterified with 1 to 4 saturated or unsaturated, linear or branched, optionally hydroxylated C₈-C₃₀ fatty acid moieties, provided they have an HLB value of more than 7, and mixtures of the above-mentioned substances, wherein the weight average HLB value of the oil-in-water emulsifier is 11-17, preferably 12-15 and particularly preferably 13-14. The ethoxylated C₈₋₂₄ alkanols have the formula R¹O(CH₂CH₂O)ₙH, wherein R¹ is a linear or branched alkyl and/or alkenyl group having 8-24 carbon atoms and n, the average number of ethylene oxide units per molecule, denotes 5-100, preferably 10-30 mol ethylene oxide per 1 mol caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and technical-grade mixtures thereof. Adducts of 5-100 mol, preferably 10-30 mol ethylene oxide, on technical-grade fatty alcohols having 12-18 carbon atoms, such as, for example, coconut, palm, palm kernel or tallow fatty alcohol, are also suitable.

Particularly preferred oil-in-water emulsifiers are selected from the group consisting of Ceteth-12, Ceteth-20, Ceteth-30, Steareth-12, Steareth-20, Steareth-30, Laureth-12 and Beheneth-20, as well as mixtures thereof. The ethoxylated C₈-C₂₄ carboxylic acids have the formula R¹(OCH₂CH₂)ₙOH wherein R¹ stands for a linear or branched saturated or unsaturated acyl group having 8-24 carbon atoms and n, the average number of ethylene oxide units per molecule, denotes 5-100 mol, preferably 10-30 mol, ethylene oxide per 1 mol caprylic acid, 2-ethylhexanoic acid, capric acid, lauric acid, isotridecanoic acid, myristic acid, cetyl acid, palmitoleic acid, stearic acid, isostearic acid, oleic acid, elaidic acid, petroselic acid, arachidic acid, gadoleic acid, behenic acid, erucic acid and brassidic acid, and technical-grade mixtures thereof. Adducts of 5-100 mol, preferably 10-30 mol, of ethylene oxide onto technical-grade fatty acids having 12-18 carbon atoms, such as coconut, palm, palm kernel or tallow fatty acids, are also suitable. Particular preference is given to PEG-50 monostearate, PEG-100 monostearate, PEG-50 monooleate, PEG-100 monooleate, PEG-50 monolaurate and PEG-100 monolaurate. Particular preference is given to using the C₁₂-C₁₈ alkanols or the C₁₂-C₁₈ carboxylic acids having in each case 10-30 units of ethylene oxide per molecule, and mixtures of these substances. In addition, C₈-C₂₂ alkyl mono- and oligoglycosides are preferably used. C₈-C₂₂ alkyl mono- and oligoglycosides constitute known standard commercial surfactants and emulsifiers. They are prepared, in particular, by reacting glucose or oligosaccharides with primary alcohols having 8-22 carbon atoms. With regard to the glycoside group, both monoglycosides in which a cyclic sugar group is bonded glycosidically to the fatty alcohol, and also oligomeric glycosides with a degree of oligomerization up to about 8, but preferably of 1-2, are suitable. The degree of oligomerization here is a statistical average value, which is based on a homologous distribution as is customary for such technical products. Products which are obtainable under the trademark Plantacare® comprise a glucosidically bonded C₈-C₁₆ alkyl group on an oligoglucoside group whose average degree of oligomerization is 1-2. Particularly preferred C₈-C₂₂ alkyl mono- and oligoglycosides are chosen from among octyl glucoside, decyl glucoside, lauryl glucoside, palmityl glucoside, isostearyl glucoside, stearyl glucoside, arachidyl glucoside and behenyl glucoside, and mixtures thereof. The acylglucamides derived from glucamine are also suitable as nonionic oil-in-water emulsifiers. Ethoxylated sterols, in particular, ethoxylated soya sterols, also represent suitable oil-in-water emulsifiers according to the invention. The degree of ethoxylation must be greater than 5, but preferably at least 10, in order to have an HLB value greater than 7. Suitable commercial products are, e.g., PEG-10 Soy Sterol, PEG-16 Soy Sterol and PEG-25 Soy Sterol. In addition, partial esters of polyglycerols having 2 to 10 glycerol units and esterified with 1 to 4 saturated or unsaturated, linear or branched, optionally hydroxylated C₈-C₃₀ fatty acid groups are preferably used, provided they have an HLB value of more than 7. Particular preference is given to diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol manocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Particularly preferred sticks according to the invention, in particular deodorant or antiperspirant sticks, are characterized in that the amount of nonionic oil-in-water emulsifier b) relative to the overall composition is 0.5-10 wt. %, preferably 0.6-4 wt. %, particularly preferably 0.7-1.5 wt. %.

Water-in-Oil Emulsifiers

The stick compositions according to the invention further comprise at least one nonionic water-in-oil emulsifier with an HLB value greater than 1.0 and less than or equal to 7.0, which can form liquid crystalline structures solely with water or with water in the presence of a hydrophilic emulsifier as a consistency regulator and/or water binder. The water-in-oil emulsifier(s) principally contribute to the constitution of the lipophilic gel phase that surrounds the dispersed lipid/wax/oil phase, as well as but to a lesser degree to the constitution of the hydrophilic gel phase that stabilizes the aqueous phase. Mainly, emulsifiers having an HLB value greater than 1 and less than or equal to 7.0 are suitable as the non-ionic water-in-oil emulsifiers. Some of these emulsifiers are listed, for example in Kirk-Othmer, “Encyclopedia of Chemical Technology”, 3rd edition, 1979, volume 8, page 913. The HLB value for ethoxylated adducts can also be calculated, as already mentioned. Preferred oil-in water emulsifiers are:
esters and, in particular, partial esters of a polyol having 2-6 carbon atoms and linear saturated and unsaturated fatty acids having 12-30, in particular 14-22, carbon atoms, which may be hydroxylated. Such esters or partial esters are, for example, the monoesters and diesters of glycerol or the monoesters of propylene glycol with linear saturated and unsaturated C₁₂-C₃₀ carboxylic acids, which may be hydroxylated, in particular those of myristic acid, palmitic acid, stearic acid or of mixtures of these fatty acids, the methylglucose mono- and diesters of linear, saturated and unsaturated C₁₂-C₃₀-carboxylic acids, which may be hydroxylated, the pentaerythrityl di-tri- and tetraesters of linear saturated and unsaturated C₁₂-C₃₀ carboxylic acids, which may be hydroxylated, of which the mono-, di-, tri- and tetraesters of pentaerythritol with linear saturated fatty acids containing 12-30, particularly 14-22 carbon atoms which can be hydroxylated, as well as mixtures thereof are particularly preferred as a consistency regulator and/or water binder. The mono- and diesters are particularly preferred according to the invention. Inventively preferred C₁₂-C₃₀ fatty acid esters are selected from lauric acid, myristic acid, palmitic acid, stearic acid, arachic acid and behenic acid moieties; the stearic acid moiety is particularly preferred. According to the invention, particularly preferred non-ionic water-in-oil emulsifiers with an HLB value greater than 1.0 and less than/equal to 7.0 are selected from pentaerythrityl monostearate, pentaerythrityl distearate, pentaerythrityl tristearate, pentaerythrityl tetrastearate, ethylene glycol monostearate, ethylene glycol distearate as well as mixtures thereof.
linear saturated alkanols containing 12-30 carbon atoms, in particular containing 16-22 carbon atoms, in particular cetyl alcohol, stearyl alcohol, arachidyl alcohol, behenyl alcohol and lanolin alcohol or mixtures of these alcohols, as are obtained from the industrial hydrogenation of vegetal and animal fatty acids,
sterols, i.e., steroids which carry a hydroxyl group on the C3 atom of the steroid backbone and are isolated both from animal tissue (zoosterols, e.g., cholesterol, lanosterol) and also from plants (phytosterols, e.g., ergosterol, stigmasterol, sitosterol) and from fungi and yeasts (mycosterols) and which may have low degrees of ethoxylation (1-5 EO);
alkanols and carboxylic acids having in each case 8-24 carbon atoms, in particular having 16-22 carbon atoms, in the alkyl group and 1-4 ethylene oxide units per molecule, which have an HLB value greater than 1.0 and less than or equal to 7.0,
glycerol monoethers of saturated and/or unsaturated, branched and/or unbranched alcohols with a chain length of 8-30, in particular 12-18, carbon atoms;
partial esters of polyglycerols having n=2 to 10 glycerol units and esterified with 1 to 5 saturated or unsaturated, linear or branched, optionally hydroxylated C₈-C₃₀ fatty acid moieties, provided they have an HLB value of less than or equal to 7,
as well as mixtures of the abovementioned substances.
   According to the invention, it may be preferred to use only one single water-in-oil emulsifier. In another preferred embodiment, the compositions according to the invention comprise mixtures, in particular technical-grade mixtures, of at least two additional water-in-oil emulsifiers, in particular mixtures of at least two water-in-oil emulsifiers of the same type (for example mixtures of only glyceryl esters or mixtures of only pentaerythrityl esters). A technical-grade mixture is understood, for example, as meaning a commercial product such as Cutina® GMS, which constitutes a mixture of glyceryl monostearate and glyceryl distearate, or a commercial product such as Cutina® PES. Inventively extremely preferred water-in-oil emulsifiers are selected from the mono- and diesters of ethylene glycol and the mono-, di-, tri- and tetraesters of pentaerythritol with linear saturated fatty acids containing 12-30, in particular 14-22 carbon atoms which can be hydroxylated, as well as mixtures thereof, which for example are obtainable as the commercial products Cutina PES (INCI: Pentaerythrityl distearate), Cutina AGS (INCI: Glycol distearate) or Cutina EGMS (INCI: Glycol stearate). These commercial products are already mixtures of mono- and diesters (tri- and tetraesters are also obtained for the pentaerythrityl esters). Inventively preferred C₁₂-C₃₀ fatty acid moieties are chosen from among lauric acid, myristic acid, palmitic acid, stearic acid, arachidic acid, and behenic acid moieties; the stearic acid group is particularly preferred. The non-ionic water-in-oil emulsifiers with an HLB value of greater than 1.0 and less than or equal to 7.0 that are particularly preferred according to the invention are chosen from among pentaerythrityl monostearate, pentaerythrityl distearate, pentaerythrityl tristearate, pentaerythrityl tetrastearate, ethylene glycol monostearate, ethyleneglycol distearate, and mixtures thereof. Additional water-in-oil emulsifiers which can be used particularly advantageously are stearyl alcohol, cetyl alcohol, glyceryl monostearate, in particular, in the form of the commercial products Cutina® GMS and Cutina® MD (ex Cognis), glyceryl distearate, glyceryl monocaprate, glyceryl monocaprylate, glyceryl monolaurate, glyceryl monomyristate, glyceryl monopalmitate, glyceryl monohydroxystearate, glyceryl monooleate, glyceryl monolanolate, glyceryl dimyristate, glyceryl dipalmitate, glyceryl dioleate, propylene glycol monostearate, propylene glycol monolaurate, sorbitan monocaprylate, sorbitan monolaurate, sorbitan monomyristate, sorbitan monopalmitate, sorbitan monostearate, sorbitan sesquistearate, sorbitan distearate, sorbitan dioleate, sorbitan sesquioleate, sucrose distearate, arachidyl alcohol, behenyl alcohol, polyethylene glycol (2) stearyl ether (Steareth-2), Steareth-5, Oleth-2, diglycerol monostearate, diglycerol monoisostearate, diglycerol monooleate, diglycerol dihydroxystearate, diglycerol distearate, diglycerol dioleate, triglycerol distearate, tetraglycerol monostearate, tetraglycerol distearate, tetraglycerol tristearate, decaglycerol pentastearate, decaglycerol pentahydroxystearate, decaglycerol pentaisostearate, decaglycerol pentaoleate, soy sterol, PEG-1 soy sterol, PEG-5 soy sterol, PEG-2 monolaurate and PEG-2 monostearate.

The water-in-oil emulsifier(s) mainly contribute(s) to the structure of the lipophilic gel phase which surrounds the dispersed lipid/wax/oil phase, as well as, albeit to a lesser extent, to the structure of the hydrophilic gel phase which stabilizes the aqueous phase. According to the invention it may be preferred to use only a single water-in-oil emulsifier. In another preferred embodiment, the compositions of the invention contain mixtures, especially technical-grade mixtures, of at least two water-in-oil emulsifiers. Particularly preferred sticks according to the invention, in particular deodorant or antiperspirant sticks, comprise the at least one nonionic water-in-oil emulsifier c) in a total amount of 0.1 to 15 wt. %, preferably 0.5 to 8.0 wt. % and particularly preferably 1 to 4 wt. %, each based on total composition. Moreover, amounts of 2 to 3 wt. %, based on the total weight of the composition, can also be extremely preferred according to the invention.

The following table contains various oil-in-water emulsifiers and water-in-oil emulsifiers and their HLB values. The HLB values, however, can also be calculated using Griffin's method, as for example in the RÖMPP Chemie Lexikon, specifically the online version of November 2003, and the handbooks from Fiedler, Kirk-Othmer, and Janistyn cited there under the keyword “HLB System.” As long as there is conflicting HLB data for a substance found in the literature, the HLB value that comes closest to Griffin's HLB value should be used for the teaching of the invention. If no clear HLB value can be determined this way, the HLB value stated by the manufacturer of the emulsifier should be used for the teaching of the invention. If that is not possible either, then the HLB is determined experimentally.

HLB value chemical designation (from Janistyn, see RÖMPP Chemie Lexicon, keyword “HLB-System”)
1 Triglycerides of saturated fatty acids

Glyceryltrioleate
1.5 Ethyleneglycol distearate
1.6 Pure cellin oil
1.8 Sorbitan trioleate

Glycerol dioleate
2.1 Sorbitan tristearate
2.4 Propylene glycol lactostearate
2.7 Glycerol monooleate

Sorbitol dioleate
2.8 Glycerol monostearate

Propylene glycol mono-/distearate, non-self-emulsifying
2.9 Ethylenglycol monostearate
3.0 Decaglycerol decaoleate

Decaglycerol decastearate

Generol 122 (Rapeseed Sterols)

Sucrose distearate
3.1 Decaglycerol decaoleate

Glyceryl monoricinoleate

Pentaerythrityl monostearate

Pentaerythrityl sesquioleate
3.2 Ethyleneglycol monodistearate, non-self-emulsifying

Glycolstearate
3.3 Glycerol monolaurate
3.4 Propylene glycol monostearate
3.5 Ethylene glycol monostearate

Pentaerythrityl monooleate

Polyethylene glycol (100)monooleate
3.6 Glycerol mono-/dioleate, non-self-emulsifying

Monoethoxylauryl ether
3.7 Sorbitan sesquioleates (Dehymuls SSO)
3.8 Glycerol monodistearate, non-self-emulsifying

Polyethylene glycol (100) monostearates

Diglycerol sesquioleates

N,N-Dimethylcaproamide

Pentaerythrityl monotallowates

Propylene glycol monolaurate
4.0 Decaglycerol octaoleate
4.3 Sorbitan monooleate (Dehymuls SMO)

Diethylene glycol monostearate
4.4 1,2-Propylene glycol monodistearate, self-emulsifying
4.5 Glycerol monostearate palmitate (90%), non-self-emulsifying

Propylene glycol monolaurate
4.7 Sorbitan monostearate (Dehymuls SMS)

Diethylene glycol monooleate
4.8 Pentaerythrityl monolaurate
4.9 Polyoxyethylene(2)oleyl alcohol (Polyoxyethylene(2)oleyl ether)

Polyoxyethylene(2)stearyl alcohol (Polyoxyethylene(2)stearyl ether)
5.0 Generol 122 E 5 (PEG-5 Soy Sterol)

Polyethylene glycol (100) monoricinoleate

Polyethylene glycol (200) distearate

Polyglyceryl-3-isostearate (e.g. Isolan GI 34 by Tego)
5.9 Polyethylene glycol (200) dilaurate
6.0 Decaglycerol tetraoleate

Polyethylene glycol (100) monolaurates

Polyethylene glycol (200) dioleate
6.1 Diethylene glycol monolaureat (diglycol laureate)
6.3 Polyethylene glycol (300) dilaurates
6.4 Glycerol monoricinoleate

Glycerol sorbitan monolaurate
6.5 Diethylene glycol monolaurate

Sodium stearoyl-2-lactylate
6.7 Sorbitan monopalmitate
6.8 Glycerol monococoate

Glycerol monolaurate
7.0 Polyoxyethylen(2)C10-C14-fatty alcohol ether, Laureth-2 (Dehydrol LS 2)

Sucrose distearate
7.2 Polyethylene glycol (400) dioleate

Sucrose dioleate
7.4 Polyethylene glycol (100) monolaurate
7.5 Sucrose dipalmitate
7.6 Glycerol sorbitan laurate
7.8 Polyethylene glycol (400) distearates
7.9 Polyethylene glycol (200) monostearate

Polyoxyethylene (3) tridecyl alcohol
8-8.2 Polyethylene glycol (400) distearate
8.0 Polyoxyethylene(3)C10-C14-fatty alcohol, Laureth-3 (Dehydrol LS 3)

N.N-Dimethyllauramide

Sodium lauroyl lactylate, sodium lauroyl-2-lactylate

Polyethylene glycol (200) monooleate

Polyethylene glycol (220) monotallowate

Polyethylene glycol (1500) dioleate

Polyoxyethylene (4) oleyl alcohol

Polyoxyethylene (4) stearylcetyl ether
8.2 Triglycerol monooleate
8.3 Diethylene glycol monolaurate
8.4 Polyoxyethylene (4) cetylether

Polyoxyethylene glycol (400) dioleate
8.5 Sodium caproyl lactylate

Polyethylene glycol (200) monostearate

Sorbitan monooleate
8.6 Sorbitan monolaurate (Dehymuls SML)

Polyethylene glycol (200) monolaurate
8.8 Polyoxyethylene (4) myristyl ether

Polyethylene glycol (400) dioleate
8.9 Nonylphenol, polyoxyethylated with 4 Mol EO
9.0 Oleth-5 (z. B. Eumulgin O 5)
9.2-9.7 Polyoxyethylene (4) lauryl alcohol (depending on the commercial product. e.g. Brij 30, Dehydrol LS 4)
9.3 Polyoxyethylene (4) tridecyl alcohol
9.6 Polyoxyethylene (4) sorbitan monostearate
9.8 Polyethylenglycol (200) monolaurate
10-11 Polyethylene glycol (400) monooleate
10.0 Didodecyldimethylammoniumchloride
10.0 Polyethylene glycol (200) monolaurate

Polyethylene glycol (400) dilaurate

Polyethylene glycol (600) dioleate

Polyoxyethylene (4) sorbitan monostearate

Polyoxyethylene (5) sorbitan monooleate
10.2 Polyoxyethylene (40) sorbitol hexaoleate
10.4-10.6 Polyoxyethylene glycol (600) distearate
10.5 Polyoxyethylene (20) sorbitan tristearate
10.6 Sucrose monostearate
10.7 Sucrose monooleate
11-11.4 Polyethylene glycol (400) monooleate
11.0 Polyethylene glycol (350) monostearate

Polyethylene glycol (400) monotalleate

Polyoxyethylene glycol (7) monostearate

Polyoxyethylene glycol (8) monooleate

Polyoxyethylene (20) sorbitan trioleate

Polyoxyethylene (6) tridecyl alcohol
11.1 Polyethylene glycol (400) monostearate
11.2 Polyoxyethylene (9) monostearate

Sucrose monooleate

Sucrose monostearate
11.4 Polyoxyethylene (50) sorbitol hexaoleate

Sucrose monotalleate

Sucrose stearate palmitate
11.6 Polyoxyethylene glycol (400) monoricinoleate
11.7 Sucrose monomyristeate

Sucrose monopalmitate
12.0 PEG-10 Soy Sterol (e.g. Generol 122 E 10)

Triethanolamine oleate
12.2-12.3 Nonylphenol, ethoxylated with 8 Mol EO
12.2 Sucrose monomyristeate
12.4 Sucrose monolaurate

Polyoxyethylene (10) oleyl alcohol, polyoxyethylene (10) oleyl ether

Polyoxyethylene (10) stearyl alcohol, polyoxyethylene (10) stearyl ether
12.5 Polyoxyethylene (10) stearylcetyl ether
12.7 Polyoxyethylene (8) tridecyl alcohol
12.8 Polyoxyethylene glycol (400) monolaurate

Sucrose monococoate
12.9 Polyoxyethylene (10) cetylether
13 Glycerol monostearate, ethoxylated (20 Mol EO)
13.0 Eumulgin O 10

Eumulgin 286

Eumulgin B 1 (Ceteareth-12)
13.0 C12-fat amines, ethoxylated (5 Mol EO)
13.1 Nonylphenol, ethoxylated (9.5 Mol EO)
13.2 Polyethylene glycol (600) monostearate

Polyoxyethylene (16) tallow oil
13.3 Polyoxyethylene (4) sorbitan monolaurate
13.5 Nonylphenol, ethoxylated (10.5 Mol EO)

Polyethylene glycol (600) monooleate
13.7 Polyoxyethylene (10) tridecyl alcohol

Polyethylene glycol (660) monotallowate

Polyethylene glycol (1500) monostearate

Polyoxyethylene glycol (1500) dioleate
13.9 Polyethylene glycol (400) monococoate

Polyoxyethylene (9) monolaurate
14-16 Eumulgin HRE 40 (castor oil, ethoxylated and hydrogenated with 40 EO)
14.0 Polyoxyethylene (12) lauryl ether

Polyoxyethylene (12) tridecyl alcohol
14.2 Polyoxyethylene (15) stearyl alcohol
14.3 Polyoxyethylene (15) stearylcetyl ether
14.4 Mixture of C12-C15-fatty alcohols with 12 mol EO
14.5 Polyoxyethylene (12) lauryl alcohol
14.8 Polyoxyethylene glycol (600) monolaurate
14.9-15.2 Sorbitan monostearate, ethoxylated with 20 EO (e.g. Eumulgin SMS 20)
15-15.9 Sorbitan monooleate, ethoxylated with 20 EO (e.g. Eumulgin SMO 20)
15.0 PEG-20 Glyceryl stearate (e.g. Cutina E 24)

PEG-40 Castor Oil (e.g. Eumulgin RO 40)

Decyl glucoside (Oramix NS 10)

Dodecyl glucoside (Plantaren APG 600)

Dodecyl trimethyl ammonium chloride

Nonylphenol, ethoxyalted with 15 Mol EO

Polyethylene glycol (1000) monostearate

Polyoxyethylene (600) monooleate
15-17 Eumulgin HRE 60 (castor oil, ethoxylated and hydrogenated with 60 EO)
15.3 C12-fatty amines, polyoxyethylated with 12 Mol EO

Polyoxyethylene (20) oleyl alcohol, polyoxyethylene (20) oleylether
15.4 Polyoxyethylene (20) stearylcetylether (z. B. Eumulgin B 2 (Ceteareth-20))
15.5 Polyoxyethylene (20) stearyl alcohol
15.6 Polyoxyethylene glycol (1000) monostearate

Polyoxyethylene (20) sorbitan monopalmitate
15.7 Polyoxyethylene (20) cetyl ether
15.9 Disodium triethanolamine distearyl heptaglycol ether sulfosuccinate
16.0 Nonylphenol ethoxylated with 20 Mol EO

Polyoxyethylene (25) propylene glycol stearate
16-16.8 Polyoxyethylene (30) monostearate
16.3-16.9 Polyoxyethylene (40) monostearate
16.5-16.7 Polyoxyethylene (20) sorbitan monolaureate (e.g. Eumulgin SML 20)
16.6 Polyoxyethylene (20) sorbitol
16.7 C18 fatty amines. polyoxyethylated with 5 Mol EO

Polyoxyethylene (23) lauryl alcohol
17.0 Ceteareth-30, z. B. Eumulgin B 3

Octyl glucoside (Triton CG 110)

Polyoxyethylene (30) glyceryl monolaurate

17.1 Nonylphenol, ethoxylated with 30 Mol EO
17.4 Polyoxyethylene (40) stearyl alcohol

Further preferred stick compositions according to the invention are wherein the total content of nonionic and ionic emulsifiers and/or surfactants with an HLB value above 8 is a maximum of 20 wt. %, a preferred maximum of 15 wt. %, a particularly preferred maximum of 10 wt. %, a particularly preferred maximum of 7 wt. %, a further particularly preferred maximum of 4 wt. %, and an exceptionally preferred maximum of 3 wt. %, referring respectively to the total composition according to the invention.

Oils

The stick compositions according to the invention further comprise at least one oil, which is liquid under normal conditions, which represents neither a fragrance component nor essential oil, wherein the (average) solubility parameter of the total of the contained oils differs by a maximum of −1.0 (cal/cm³)^{0.5} resp. a maximum of +1.0 (cal/cm³)^{0.5}, preferably by a maximum of −0.8 (cal/cm³)^{0.5} resp. a maximum of +0.8 (cal/cm³)^{0.5}, particularly preferably by a maximum of −0.7 (cal/cm³)^{0.5} resp. a maximum of +0.7 (cal/cm³)^{0.5}, extremely preferably by a maximum of −0.6 (cal/cm³)^{0.5} resp. a maximum of +0.4 (cal/cm³)^{0.5} from the (average) solubility parameter of the water-in-oil emulsifier(s).

The matching of the used oil(s) with the used water-in-oil emulsifier(s) represents an important parameter of this invention. If the water-in-oil emulsifiers and the oil component(s) do not match each other in their solubility parameter within the required limits, one will obtain sticks with an unsatisfactory degree of hardness and stability from the point of view of usage. Preferred oils according to the invention are chosen from the benzoic acid esters of linear or branched C₈₋₂₂ alkanols. The C₁₂-C₁₅ alkyl esters of benzoic acid, e.g. available as the commercial product Finsolv®TN, the isostearyl ester of benzoic acid, e.g. available as the commercial product Finsolv® SB, ethylhexyl benzoate, e.g. available as the commercial product Finsolv® EB, and the octyldodecyl ester of benzoic acid, e.g. available as the commercial product Finsolv® BOD, are particularly preferred. Further preferred oils according to the invention are selected from branched saturated or unsaturated fatty alcohols containing 6-30 carbon atoms. These alcohols are often also referred to as Guerbet Alcohols, as they are obtained by the Guerbet Reaction. Preferred alcohol oils are hexyldecanol (Eutanol® G 16, Guerbitol® T 16), octyldodecanol (Eutanol® G, Guerbitol® 20), 2-ethylhexyl alcohol and the commercial products Guerbitol® 18, Isofol® 12, Isofol® 16, Isofol® 24, Isofol® 36, Isocarb® 12, Isocarb® 16 or Isocarb® 24. Further preferred oil components are mixtures of Guerbet Alcohols and Guerbet Alcohol esters, for example the commercial product Cetiol® PGL (hexyldecanol and hexyldecyl laurate).

Further preferred oils according to the invention are chosen from the triglycerides of linear or branched, saturated or unsaturated, optionally hydroxylated C₈₋₃₀ fatty acids. The use of natural oils e.g. Soya oil, cottonseed oil, sunflower oil, palm oil, palm seed oil, linseed oil, Almond oil, castor oil, corn oil, olive oil, rapeseed oil, sesame seed oil, safflower oil, wheat germ oil, peach seed oil and the liquid parts of coconut oil and the like can be particularly suitable. However, synthetic triglycerides are also suitable, in particular capric/caprylic triglycerides, e.g. the commercial products Myritol® 318, Myritol® 331 (Cognis) or Miglyol® 812 (Hüls) with non-branched fatty acid moieties as well as glyceryl tri-isostearine and the commercial products Estol® GTEH 3609 (Uniqema) or Myritol® GTEH (Cognis) with branched fatty acid moieties. Further particularly preferred oils according to the invention are chosen from the dicarboxylic acid esters of linear or branched C₂-C₁₀ alkanols, in particular di-isopropyl adipate, di-n-butyl adipate, di-(2-ethylhexyl) adipate, dioctyl adipate, diethyl-/di-n-butyl/dioctyl sebacate, di-isopropyl sebacate, dioctyl malate, dioctyl maleate, dicaprylyl maleate, di-isooctyl succinate, di-2-ethylhexyl succinate and di-(2-hexyldecyl) succinate. Further particularly preferred oils according to the invention are chosen from the addition products of 1 to 5 propylene oxide units onto monohydric or polyhydric C₈₋₂₂ alkanols such as octanol, decanol, decanediol, lauryl alcohol, myristyl alcohol and stearyl alcohol, e.g. PPG-2-myristyl ether and PPG-3-myristyl ether (Witconol® APM).

It can be inventively extremely preferred to employ mixtures of the cited oils in order to achieve an optimal fine match of the stick properties, such as stick hardness, residue behavior, abrasion properties or active ingredient release.

Preferred sticks according to the invention, in particular deodorant or antiperspirant sticks, are characterized in that the oil d) that is liquid under normal conditions is selected from benzoic acid esters of linear or branched C₈-C₂₂ alkanols, of branched saturated or unsaturated fatty alcohols containing 6-30 carbon atoms, triglycerides of linear or branched, saturated or unsaturated, optionally hydroxylated C₈-C₃₀ fatty acids, dicarboxylic acid esters of linear or branched C₂-C₁₀ alkanols, esters of branched saturated or unsaturated fatty alcohols containing 2-30 carbon atoms with linear or branched saturated or unsaturated fatty acids containing 2-30 carbon atoms which can be hydroxylated, addition products of 1 to 5 propylene oxide units onto mono- or polyhydric C₈₋₂₂ alkanols, addition products of at least 6 ethylene oxide and/or propylene oxide units to monohydric or polyhydric C₃₋₂₂ alkanols, C₈-C₂₂ fatty alcohol esters of monovalent or polyvalent C₂-C₇ hydroxycarboxylic acids, symmetric, asymmetric or cyclic esters of carbonic acid with fatty alcohols, the esters of dimerized unsaturated C₁₂-C₂₂ fatty acids (dimerized fatty acids) with monohydric linear, branched or cyclic C₂-C₁₈ alkanols or with polyhydric linear or branched C₂-C₆ alkanols, as well as mixtures of the substances cited above.

Particularly preferred sticks according to the invention, in particular deodorant or antiperspirant sticks, are wherein the oil(s) d), which is/are liquid under normal conditions is/are comprised in a total amount of 3-20 wt. %, preferably 5-14 wt. %, particularly preferably 6-12 wt. %, relating respectively to the total weight of the composition.

In a further particularly preferred version of the invention, the fraction of oil(s), whose solubility parameter differs by more than −1.0 (cal/cm³)^{0.5} resp. more than +1.0 (cal/cm³)^{0.5} from (the average) solubility parameter of the water-in-oil emulsifier(s), is a maximum of 20 wt. % in relation to the total weight of oils, which are liquid under normal conditions. In a further particularly preferred version of the invention no such oils are contained, which are liquid under normal conditions, the solubility parameter of which differs by more than ±1.2 (cal/cm³)^{0.5} from (the average) solubility parameter of the water-in-oil emulsifier(s). Corresponding less suitable or (depending on the water-in-oil emulsifier used) even unsuitable oil components are for example silicone oils and hydrocarbon oils. Silicon oils, among which are e.g. dialkyl- and alkylarylsiloxanes, such as for example not only cyclopentasiloxane, cyclohexasiloxane, dimethylpolysiloxane and methylphenylpolysiloxane, but also hexamethyldisiloxane, octamethyltrisiloxane and decamethyltetrasiloxane, display solubility parameters in the range of around 5.7 to 6.3 (cal/cm³)^{0.5}, which is a divergence of more than 1.2 (cal/cm³)^{0.5} of the value of most of the inventively used water-in-oil emulsifiers. Natural and synthetic hydrocarbons such as paraffin oils, isohexadecane, isoeicosane, polyisobutene or polydecene, which are available for example under the name Emery® 3004, 3006, 3010 or under the name Ethylflo® from Albemarle or Nexbase® 2004G from Nestle, as well as 1,3-di-(2-ethylhexyl)cyclohexane (Cetiol®S) are similarly among the inventively less preferred oil components. The fraction of silicon oils and/or hydrocarbons in a preferred embodiment of the invention should therefore not be more than 20% in relation to the total weight of oils, which are liquid under normal conditions, otherwise the sticks according to the invention do not achieve the desired hardness and stability when used. In a particularly preferred embodiment of the invention no silicon oils and/or hydrocarbons, in particular no paraffinic and iso-paraffinic hydrocarbons are comprised.

Polyols

The stick compositions according to the invention additionally comprise at least one water-soluble polyhydric C₂-C₉ alkanol having 2-6 hydroxyl groups and/or at least one water-soluble polyethylene glycol containing 3-20 ethylene oxide units, as well as mixtures thereof. These components are preferably chosen from 1,2-propylene glycol, 2-methyl-1,3-propanediol, glycerol, butylene glycols such as 1,2-butylene glycol, 1,3-butylene glycol and 1,4-butylene glycol, pentylene glycols such as 1,2-pentanediol and 1,5-pentanediol, hexanediols, such as 1,6-hexanediol, hexanetriols such as 1,2,6-hexanetriol, 1,2-octanediol, 1,8-octanediol, dipropylene glycol, tripropylene glycol, diglycerine, triglycerine, erythritol, sorbitol, xyltol as well as mixtures of the cited substances. Suitable water soluble polyethylene glycols are chosen from PEG-3, PEG-4, PEG-6, PEG-7, PEG-8, PEG-9, PEG-10, PEG-12, PEG-14, PEG-16, PEG-18 and PEG-20, as well as mixtures thereof, wherein PEG-3 to PEG-8 are preferred. Also sugar and certain sugar derivatives such as fructose, glucose, maltose, maltitole, mannite, inosite, sucrose, trehalose and xylose are suitable according to the invention.

Preferred sticks according to the invention, in particular deodorant or antiperspirant sticks, are wherein the at least one water soluble polyhydric C₂-C₉ alkanol with 2-6 hydroxyl groups and/or at least one water soluble polyethylene glycol with 3-20 ethylene oxide units is chosen from 1,2-propylene glycol, 2-methyl-1,3-propanediol, glycerine, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, dipropylene glycol, tripropylene glycol, diglycerine, triglycerine, erythritol, sorbitol as well as mixtures of the substances named above.

Particularly preferred sticks according to the invention, in particular deodorant or antiperspirant sticks are wherein the at least one water soluble polyhydric C₂-C₉ alkanol with 2-6 hydroxyl groups and/or at least one water soluble polyethylene glycol with 3-20 ethylene oxide units is contained in all in quantities of 3-30 wt. %, preferably 8-25 wt. %, particularly preferably 10-18 wt. %, related respectively to the total composition.

Water

The fraction of water in the composition according to the invention is 5 to 70 wt. %, preferably 10 to 35 wt. %, particularly preferably 15-30 wt. %, exceptionally preferably 20-28 wt. %, relating respectively to the total composition.

Preferred stick compositions according to the invention comprise at least one deodorant and/or antiperspirant active substance as the cosmetic active ingredient.

Deodorant Substances

Deodorant substances preferred according to the invention are odor absorbers, de-odorizing ionic exchangers, germ inhibiting agents, pre-biotic components as well as enzyme inhibitors or, particularly preferably, combinations of the cited substances. Silicates serve as odor absorbers, which simultaneously advantageously support the rhelogical characteristics of the composition according to the invention. Among the particularly advantageous silicates according to the invention are above all layered silicates and among these in particular montmorillonite, kaolinite, ilite, beidellite, nontronite, saponite, hectorite, bentonite, smectite and talcum. Further advantageous odor absorbers are for example zeolites, zinc ricinoleate, cyclodextrine, and certain metallic oxides such as e.g. aluminum oxide as well as chlorophyll. They are preferably used in a quantity of 0.1-10 wt. %, particularly preferred 0.5-7 wt. % and exceptionally preferred 1-5 wt. %, relating respectively to the total composition. Germ inhibiting or anti-microbial substances in the context of the invention are understood to mean such substances, which reduce the number resp. the growth of odor engendering germs inhabiting the skin. These germs include among others various species of the group of the staphylococci, and of the groups coryne bacteria, anaerococci and micrococci. Preferred as germ inhibiting or anti-microbial substances according to the invention are in particular organo-halogen compounds as well as halogenides, quaternary ammonium compounds, a series of vegetal extracts and zinc compounds. These include among others triclosan, chlorhexidine and chlorhexidine gluconate, 3,4,4′-trichlorocarbanilide, bromochlorophene, dichlorophene, chlorothymol, chloroxylene, hexachlorophene, dichloro-m-xylene, dequalinium chloride, domiphene bromide, ammonium phenolsulfonate, benzalkonium halogenides, benzalkonium cetylphosphate, benzalkonium saccharinate, benzethonium chloride, cetylpyridinium chloride, laurylpyridinium chloride, laurylisoquinolinium bromide, methylbenzedonium chloride. Further usable are phenol, phenoxyethanol, disodium dihydroxy-ethylsulfo-succinylundecylenate, sodium bicarbonate, zinc lactate, sodium phenolsulfonate and zinc phenolsulfonate, ketoglutaric acid, terpene alcohols such as e.g. farnesol, chlorophylline copper complexes, α-mono-alkylglycerine ethers with a branched or linear saturated or unsaturated, optionally hydroxylated C₆-C₂₂ alkyl group, particularly preferably α-(2-ethylhexyl) glycerine ethers, commercially available as Sensiva® SC 50 (ex Schülke & Mayr), carboxylic acid esters of the mono-, di- and tri glycerines (e.g. glycerine monolaurate, diglycerine monocaprinate), lantibiotics as well as vegetal extracts (e.g. green tea and parts of linden blossom oil). Further preferred deodorant substances are chosen from so-called prebiotic components, by which such components in the context of the invention are understood to mean that they inhibit only or at least preponderantly the odor engendering germs of the skin microflora, but not the desired, i.e. the non-odor engendering germs, which belong to healthy skin flora. Explicitly included here are the substances that are described in the publications DE 10333245 and DE 10 2004 011 968 as prebiotically active; among these are coniferous extracts, in particular of the group of the pinaceae, and vegetal extracts of the group of the Sapindaceae, Araliaceae, Lamiaceae and Saxifragaceae, in particular extracts of Picea spp., Paullinia sp., Panax sp., Lamium album or Ribes nigrum as well as mixtures of these substances. Further preferred deodorant substances are chosen from the germ inhibiting perfume oils and the deosafe perfumed oils, which are available from the Symrise Company, formerly Haarmann and Reimer. Among the enzyme inhibitors are substances that inhibit the enzymes responsible for the decomposition of sweat, in particular the arylsulfatase, β-glucuronidase, aminoacylase, esterases, lipases and/or lipoxigenase e.g. trialkyl esters of citric acid, in particular triethyl citrate, or zinc glycinate. Preferred sticks according to the invention, in particular deodorant or antiperspirant sticks, are wherein at least one deodorant active substance is chosen from arylsulfatase inhibitors, β-glucuronidase inhibitors, aminoacylase inhibitors, esterase inhibitors, lipase inhibitors and lipoxigenase inhibitors, α-monoalkylglycerine ethers with one branched or linear saturated or unsaturated, optionally hydroxylated C₆-C₂₂-alkyl group, in particular α-(2-ethylhexyl) glycerine ether, phenoxyethanol, germ inhibiting perfume oils, deosafe perfume oils, prebiotic components, trialkyl esters of citric acid, in particular triethyl citrate, substances that reduce the number of odor generating skin bacteria of the group of staphylococci, coryne bacteria, anaerococci and micrococci resp. inhibit their growth, zinc compounds, in particular zinc phenolsulfonate and zinc ricinoleate, organo-halogen compounds, in particular triclosan, chlorhexidine, chlorhexidine gluconate and benzalkonium halides, quaternary ammonium compounds, in particular cetylpyridinium chloride, odor absorbers, in particular silicates and zeolites, sodium bicarbonate, lantibiotics, as well as mixtures of the substances mentioned above.

Further preferred sticks according to the invention, in particular deodorant or antiperspirant sticks, comprise the at least one deodorant active substance in a total quantity of 0.1-10 wt. %, preferably 0.2-7 wt. %, particularly preferably 0.3-5 wt. % and exceptionally preferably 0.4-1.0 wt. %, related to the total weight of the active substance in the total composition.

Antiperspirant Active Substances

Preferred sticks according to the invention, in particular deodorant or antiperspirant sticks, comprise at least one antiperspirant active substance, chosen from the water soluble astringent inorganic and organic salts of aluminum, zirconium and zinc resp. desired mixtures of these salts. Particularly preferred antiperspirant active substances are chosen from aluminum chlorhydrates, in particular the aluminum chlorhydrates with the general formula [Al₂(OH)₅Cl▪2-3H₂O]ₙ that can exist in the non-active or the active (depolymerized) form, further aluminum sesquichlorohydrate, aluminum chlorhydrex-propylene glycol (PG) or -polyethylene glycol (PEG), aluminum sesquichlorhydrex-PG or —PEG, aluminum-PG-dichlorhydrex or aluminum-PEG-dichlorhydrex, aluminum hydroxide, further chosen from the aluminum zirconium chlorohydrates, such as aluminum zirconium trichlorohydrate, aluminum zirconium tetrachlorohydrate, aluminum zirconium pentachlorohydrate, aluminum zirconium octachlorohydrate, the aluminum-zirconium-chlorohydrate-glycine complexes such as aluminum zirconium trichlorohydrexglycine, aluminum zirconium tetrachlorohydrexglycine, aluminum zirconium pentachlorohydrexglycine, aluminum zirconium octachlorohydrexglycine, potassium aluminum sulfate (KAI(SO₄)₂▪12H₂O, alum), aluminum undecylenoyl collagen amino acid, sodium aluminum lactate+aluminum sulfate, sodium aluminum chlorohydroxylactate, aluminum bromohydrate, aluminum chloride, the complexes of zinc and sodium salts, the complexes of lanthanum and cerium, the aluminum salts of lipo amino acids, aluminum sulfate, aluminum lactate, aluminum chlorohydroxyallantoinate, sodium-aluminum chlorohydroxy lactate, zinc chloride, zinc sulfocarbolate, zinc sulfate and zirconium chlorohydrate. In the context of the invention, “water solubility” is understood to mean a solubility of at least 5 wt. % at 20° C., i.e. that means that quantities of at least 5 g of the antiperspirant active substances are soluble in 95 g water at 20° C. The antiperspirant active substances can be used as aqueous solutions. Particularly preferred sticks according to the invention, in particular deodorant or antiperspirant sticks, comprise at least one antiperspirant active substance in a quantity of 3%-27 wt. %, preferably 5%-22 wt. % and in particular 10%-20 wt. %, related to the total weight of the active substance in the total composition. In a particularly preferable version the composition comprises an astringent aluminum salt, in particular aluminum chlorohydrate, which for example is sold in powder form as Micro Dry® Ultrafine from Reheis, in the form of an aqueous solution as Locron® L from Clariant, as Chlorhydrol®, as well as in activated form as Reach® 501 from Reheis. An aluminum sesquichlorohydrate from Reheis is offered under the name Reach® 301, which is also particularly preferred. Also the use of aluminum zirconium tetrachlorohydrex-glycine complexes, which for example are commercially available under the name Rezal® 36G, can be particularly preferred in the context of the invention. In a further particularly preferred embodiment, the stick compositions according to the invention can comprise, at least one deodorant as well as also at least one antiperspirant active substance.

Low Melting Point Wax Components

Particularly preferred sticks according to the invention, in particular deodorant or antiperspirant sticks, comprise at least one lipid or wax component with a melting point in the range of 25-<50° C., chosen from coconut fatty acid glycerine mono-, di- and tri-esters, butyrospermum parkii (Shea Butter) and esters of saturated, monohydric C₈-C₁₈ alcohols with saturated C₁₂-C₁₈ monocarboxylic acids, as well as mixtures of these substances. These low melting point wax components enable the consistency of the product to be optimized and the visible residues on the skin to be minimized. Particularly preferred are commercial products with the INCI designation Cocoglycerides, in particular the commercial products Novata® (ex Cognis), particularly preferably Novata® AB, a mixture of C₁₂-C₁₈ mono-, di- and triglycerides, which melts in the range of 30-32° C., as well as the products of the Softisan line (Sasol Germany GmbH) with the INCI designation Hydrogenated Cocoglycerides, in particular Softisan 100, 133, 134, 138, 142. Further preferred esters of saturated, monohydric C₁₂-C₁₈ alcohols with saturated C₁₂-C₁₈ monocarboxylic acids are stearyl laurate, cetearyl stearate (e.g. Crodamol® CSS), cetyl palmitate (e.g. Cutina® CP) and myristyl myristate (e.g. Cetiol® MM).

Further particularly preferred sticks according to the invention, in particular deodorant or antiperspirant sticks, comprise at least one wax component with a melting point in the range of 25-<50° C. in quantities of 0.01 to 20 wt. %, preferably 3-20 wt. %, particularly preferably 5-18 wt. % and an exceptionally preferred 6-15 wt. %, related to the total composition.

Fillers

Particularly preferred sticks according to the invention, in particular deodorant or antiperspirant sticks, further comprise at least one solid, water-insoluble, particulate filler for the improvement of the consistency of the stick and the sensory characteristics. In an exceptionally preferred embodiment, this filler is chosen from starches, which may be modified optionally (e.g. of corn, rice, potatoes) and starch derivatives, which are pre-gelatinized if desired, in particular aluminium starch octenyl succinate, available under the name DRY FLO®, and similar starch derivatives, cellulose and cellulose derivatives, silicon dioxide, silicic acids, e.g. Aerosil®-types, spherical polyalkyl sesquisiloxane particles (in particular Aerosil® R972 and Aerosil® 200V from Degussa), silicic gels or silica, talcum, kaolin, clays, e.g. bentonites, magnesium aluminum silicates, boronitride, lactoglobuline derivatives, e.g. sodium C₈₋₁₆ isoalkylsuccinyl lactoglobulin sulfonate, available from Brooks Industries as the commercial product Biopol® OE, glass powders, polymer powders, in particular of polyolefins, polycarbonates, polyurethanes, polyamides, e.g. nylon, polyesters, polystyrenes, polyacrylates, (meth)acrylate- or (meth)acrylate-vinylidene-copolymers, which can be cross-linked, or silicones, as well as mixtures of these substances. Polymer powders based on a polymethacrylate copolymer are available, for example as the commercial product Polytrap® 6603 (Dow Corning). Other polymer powders, e.g. based on polyamides, are available under the name Orgasol® 1002 (polyamide-6) and Orgasol® 2002 (polyamide-12) from Elf Atochem. Further polymer powders that are suitable for the purposes of the invention are, for example, polymethacrylate (Micropeari® M from SEPPIC or Plastic Powder A from NIKKOL), styrene-divinylbenzene copolymers (Plastic Powder FP from NIKKOL), polyethylene and polypropylene powders (ACCUREL® EP 400 from AKZO) or also silicone polymers (silicone powder X2-1605 from Dow Corning). Particularly preferred sticks according to the invention, in particular deodorant or antiperspirant sticks, comprise at least one solid, water-insoluble, particulate filler in a total quantity of 0.01 to 30 wt. %, preferably 5-20 wt. %, particularly preferably 8 to 15 wt. %, relating respectively to the total composition.

Fragrances

Particularly preferred sticks according to the invention, in particular deodorant or antiperspirant sticks, further comprise at least one fragrance component. Perfumes, perfume oils or perfume oil constituents can be used as the fragrance components. According to the invention, perfume oils and fragrances can be single odoriferous compounds, e.g. the synthetic products of the type esters, ethers, aldehydes, ketones, alcohols and hydrocarbons. Odoriferous compounds of the ester type are e.g. benzyl acetate, phenoxyethyl isobutyrate, p-tert.-butylcyclohexyl acetate, linalyl acetate, dimethylbenzylcarbinyl acetate (DMBCA), phenylethyl acetate, benzyl acetate, ethylmethylphenyl glycinate, allylcyclohexyl propionate, styrallyl propionate, benzyl salicylate, cyclohexyl salicylate, floramate, melusate and jasmecyclate. Among the ethers are for example benzyl ethyl ether and ambroxan, among the aldehydes e.g. the linear alkanals with 8-18 C atoms, citral, citronellal, citronellyloxy-acetaldehyde, cyclamenaldehyde, lilial and bourgeonal, among the ketones e.g. the ionones, alpha-isomethyl ionone and methyl cedryl ketone, among the alcohols anethol, citronellol, eugenol, geraniol, linalool, phenylethyl alcohol and terpineol, among the hydrocarbons primarily the terpenes such as lemons and pines. Mixtures of various odoriferous substances are preferably used, which together orchestrate an appealing fragrant note. Such perfume oils can also contain natural mixtures of odoriferous substances, such as are available from vegetal sources, e.g. pine, citrus, jasmine, patchouli, rose or ylang-ylang oil. Likewise suitable are muscatel salve oil, chamomile oil, carnation oil, melissa oil, mint oil, cinnamon leaf oil, linden blossom oil, juniper berry oil, vetiver oil, olibanum oil, galbanum oil, labdanum oil, as well as orange blossom oil, neroli oil, orange peel oil and sandalwood oil. In order to be discernable, an odoriferous substance must be volatile, wherein along with the nature of the functional groups and the structure of the chemical compound, the molar mass also plays an important role. For this reason most of the odoriferous substances possess molar masses up to approximately 200 Daltons, while molar masses of 300 Daltons and above represent more of an exception. On the basis of the differing volatilities of odoriferous substances, the odor of a perfume or fragrance composed of several odoriferous substances changes during the evaporation, wherein the odoriferous impressions are divided into the “top note,” “middle note resp. body” and the “end note resp. dry out.” Since the perception of smell depends to a large extent on the intensity of the odor, the top note of a perfume resp. fragrance does not solely consist alone of highly volatile compounds, while the end note consists for the most part of less volatile i.e. more enduring odoriferous substances. In the composition of a perfume, more easily volatile odoriferous substances can be bound for example to certain fixatives, through which their too rapid evporization is hindered. In the following classification of odorous substances in “more highly volatile” resp. “enduring” odorous substances, nothing is said about the impression of the odor and about whether the corresponding odoriferous substance is perceived as the top or body note. Enduring odoriferous substances that can be employed in the context of the present invention are, for example, the ethereal oils such as angelica radix oil, aniseed oil, arnica blossom oil, basil oil, bay oil, bergamot oil, champak blossom oil, fir oil, turpentine oil, elemi oil, eucalyptus oil, fennel oil, pine needle oil, galbanum oil, geranium oil, ginger grass oil, guaiac wood oil, balsam of gurjun oil, helichrysum/chasteweed oil, ho oil, ginger oil, iris oil, cajeput oil, calmus oil, chamomile oil, camphor oil, canaga oil, cardamom oil, cassia oil, fir cone oil, balsam of kopaiva oil, coriander oil, crisped mint oil, caraway oil, cumin oil, lavender oil, lemon grass oil, lime oil, tangerine oil, melissa oil, ambrette oil, myrrh oil, carnation oil, neroli oil, niaouli oil, oliban oil, orange oil, origanum oil, palmarosa oil, patchouli oil, peru balsam oil, petit grain oil, peppermint oil, pimento oil, pine oil, rose oil, rosemary oil, sandal wood oil, celery oil, lavender oil, star anise oil, turpentine oil, thuja oil, thyme oil, verbena oil, vetiver oil, juniper berry oil, absinthe oil, winter green oil, ylang-ylang oil, hyssop oil, cinnamon oil, cinnamon leaf oil, citronella oil, lemon oil and cypress oil.

In the context of the present invention however, the higher boiling or solid odoriferous substances of natural or synthetic origin can be used as the enduring odoriferous substances resp. mixtures of odoriferous substances, i.e. fragrances. These compounds include the compounds cited below as well as mixtures thereof: ambrettolide, α-amylcinnamaldehyde, anethol, anisaldehyde, anise alcohol, anisol, methyl anthranilate, acetophenone, benzylacetone, benzaldehyde, ethyl benzoate, benzophenone, benzyl alcohol, benzyl acetate, benzyl benzoate, benzyl formate, benzyl valerianate, borneol, bornyl acetate, α-bromostyrene, n-decylaldehyde, n-dodecylaldehyde, eugenol, eugenol methyl ether, eucalyptol, farnesol, fenchone, fenchyl acetate, geranyl acetate, geranyl formate, heliotropine, heptine carboxylic acid methyl ester, heptaldehyde, hydroquinone dimethyl ether, hydroxycinnamic aldehyde, hydroxycinnamic alcohol, indole, iron, isoeugenol, isoeugenol methyl ether, isosafrol, jasmone, camphor, carvacrol, carvone, p-cresol methyl ether, cumarin, p-methoxyacetophenone, methyl n-amyl ketone, methyl methylanthranilate, p-methylacetophenone, methylchavikol, p-methylquinoline, methyl β-naphthyl ketone, methyl-n-nonylacetaldehyde, methyl n-nonyl ketone, muskone, β-naphthol ethyl ether, β-naphthol methyl ether, nerol, nitrobenzene, n-nonylaldehyde, nonyl alcohol, n-octylaldehyde, p-oxy-acetophenone, pentadecanolide, β-phenylethyl alcohol, phenylacetaldehyde dimethyl acetal, phenylacetic acid, pulegone, safrol, salicylic acid isoamyl ester, salicylic acid methyl ester, salicylic acid hexyl ester, salicylic acid cyclohexyl ester, santalol, skatol, terpineol, thyme, thymol, γ-undelactone, vanilline, veratrumaldehyde, cinnamic aldehyde, cinnamic alcohol, cinnamic acid, cinnamic acid ethyl ester, cinnamic acid benzyl ester. The more volatile odoriferous substances include the lower boiling odoriferous substances of natural or synthetic origin, which can be used alone or in mixtures. Examples of more volatile odoriferous substances are alkyl isothiocyanates (alkyl mustard oils), butanedione, lemons, linalool, linayl acetate and -propionate, menthol, menthone, methyl n-heptenone, phellandrene, phenylacetaldehyde, terpinyl acetate, citral, citronellal. Particularly preferred sticks according to the invention, in particular deodorant or antiperspirant sticks, comprise at least one fragrance component in a total quantity of 0.00001 to 4 wt. %, preferably 0.5-2 wt. %, relating respectively to the total composition.

Penetration Force Values

In a further particularly preferred embodiment, the stick compositions according to the invention are characterized by a penetration force value in the range of 150-800 gram force (g-force), preferably in the range of 250-700 gram force (g-force), particularly preferably in the range of 350-650 gram force (g-force), at a penetration depth of 5.000 mm. The penetration force value represents a measure for the hardness of a stick (or even of a solid cream composition) and states with which maximum force a defined measuring probe, here a cone of stainless steel with 45° (Model TA 15), is thrust vertically (axially) into the stick compound to be measured up to a penetration depth of 5.000 mm (five point zero zero zero mm) with a penetrative speed of 2 mm/second. The measurement of the penetration force value is carried out with the TA-XT2i Texture Analyzer of the firm Stable Micro Systems (Vienna Court, Lammas Road, Godalming, Surrey GU7 1YL, England). The maximum force is shown in gram force (g-force). Here lower values characterize a softer composition; harder compositions have a higher penetration force value. Cream-type compositions are often measured with a penetration depth of 10.000 mm (ten point zero zero zero mm), in order to obtain more exact values. This depth of penetration of the harder stick compounds usually cannot be measured since in this case the stick compound often begins to fracture. A doubling of the penetration depth means approximately a trebling up to a quadrupling of the measured value of the maximum force. The measurements are carried out under ambient conditions of 30° C. and 50% relative humidity; the specimen temperature is 23° C. The measurements are preferably carried out 3 days and/or 4 weeks after the manufacture of the stick according to the invention. The antiperspirant creams disclosed in DE 199 62 878 A1 and DE 199 62 881 A1 display penetration force values of 9-15 gram force (g-force) under the measuring conditions cited here.

Electrical Resistance

The prior art water-containing sticks are almost exclusively in the form of water-in-oil emulsions or emulsions with the aqueous phase as the dispersed phase. In order to clearly and unequivocally delimit the sticks according to the invention from the prior art, the measurement of the electrical resistance serves as a quick and reliable test, as is usual in the examinations of emulsions. Due to the continuous water phase, an oil-in-water system exhibits a higher electrical conductivity and correspondingly a lower electrical resistance than a water-in-oil system. In a further particularly preferred embodiment, the stick compositions according to the invention are characterized by an electrical resistance of maximum 400 kΩ. An electrical resistance of maximum 350 kΩ is preferred, particularly preferably a maximum of 300 kΩ. The resistance is measured with a Voltcraft model VC820 multimeter with an automatic measuring range conversion (0-400 Ω/40MΩ(±1%+2dgt)) and two micro-tipped measuring probes of 1.0 mm stainless steel. The distance between the electrodes is fixed by a millimeter gauge. The measurement is carried out at room temperature (22° C.). For this the micro-tipped electrodes are fixed parallel at a distance of 27.0 mm on the millimeter gauge and are connected to the resistance-measuring device. The measurement of the electrical resistance takes place directly on the water-containing antiperspirant sticks. For this the usually curved surface of the antiperspirant sticks is cleared away with a knife to the extent that a flat cross section results. Immediately following this the measuring electrodes are stuck vertically approximately 5 mm into the stick compound. The measured values of the electrical resistance are read off after 30 seconds. The measuring electrodes are cleaned with a cellulose cloth soaked in alcohol. Under the cited measuring conditions, tap water displays an electrical resistance of 250 kΩ, a 20 wt. % aqueous aluminum chlorohydrate solution 3 kΩ and fully desalinated water 1.7 MΩ.

Further Active Ingredients

Particularly preferred sticks according to the invention, in particular deodorant or antiperspirant sticks, further comprise pigments, e.g. titanium dioxide. The pigment content supports the cosmetic acceptance of the preparation on the part of the user. Further particularly preferred sticks according to the invention, in particular deodorant or antiperspirant sticks, comprise the usual ingredients of cosmetic preparations, e.g. colorants, nanospheres, preservatives and light stabilizers, antioxidants, enzymes as well as conditioners. Particularly preferred sticks according to the invention, in particular deodorant or antiperspirant sticks, preferably comprise them in an amount of 0.001-20 wt. %.

Product Stabilization

Particularly preferred sticks according to the invention, in particular deodorant or antiperspirant sticks, comprise at least one radical scavenger for the purposes of product stabilization, particularly preferably a substance with the INCI designation Tris(tetramethyl-hydroxy-piperidinol) citrate, which for example is available under the commercial name Tinogard Q of the firm Ciba. Tris(tetramethylhydroxy-piperidinol) citrate is preferably comprised in quantities of 0.01-0.1%, particularly preferably 0.025-0.05 wt. %, relating to the total weight of the composition according to the invention.

Further particularly preferred sticks according to the invention, in particular deodorant or antiperspirant sticks, comprise at least one UV-filter. Here the UV filters are preferably chosen from benzotriazole derivatives, in particular 2,2′-methylene-bis-(6-(2H-benzotriazole-2-yl)-4-(1,1,3,3-tetramethylbutyl)-phenol) [Tinosorb M (Ciba)], 2,2′-methyl-bis-[6-(2H-benzotriazole-2-yl)-4-(methyl)phenol] (MIXXIM BB/200 of the firm Fairmount Chemical), 2-(2′-hydroxy-3′,5′-di-t-amyl phenyl)benzotriazole (CAS-No.: 025973-551), 2-(2′-hydroxy-5′-octylphenyl)benzotriazole (CAS-No. 003147-75-9), 2-(2′-hydroxy-5′-methylphenyl)benzotriazole (CAS-No. 2440-22-4), 2-(2H-benzotriazole-2-yl)-4-methyl-6-[2-methyl-3-[1,3,3,3-tetramethyl-1-((trimethylsilyl)oxy]disiloxanyl)propyl]-phenol (CAS-No.: 155633-54-8) with the INCI designation Drometrizole trisiloxane, 2,4-bis-{[4-(2-ethyl-hexyloxy)-2-hydroxy]-phenyl}-6-(4-methoxyphenyl)-1,3,5-triazine (INCI: bis-ethylhexyloxyphenol methoxyphenyl triazine or also aniso triazine, available as Tinosorb® S from CIBA), 2,4-bis-{[4-(3-sulfonato)-2-hydroxy-propyloxy)-2-hydroxy]-phenyl}-6-(4-methoxyphenyl)-1,3,5-triazine-sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethyl-hexyloxy)-2-hydroxy]-phenyl}-6-[4-(2-methoxyethylcarboxyl)-phenylamino]-1,3,5-triazine, 2,4-bis-{[4-(3-(2-propyloxy)-2-hydroxy-propyloxy)-2-hydroxy]-phenyl}-6-[4-(ethylcarboxyl)-phenylamino]-1,3,5-triazine, 2,4-bis-{[4-(2-ethylhexyloxy)-2-hydroxy]-phenyl}-6-(1-methyl-pyrrol-2-yl)-1,3,5-triazin, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2-methylpropenyloxy)-2-hydroxy]-phenyl}-6-(4-methoxyphenyl)-1,3,5-triazine, 2,4-bis-{[4-(1′,1′,1′,3′,5′, 5′, 5′-heptamethylsiloxy-2-methyl-propyloxy)-2-hydroxy]-phenyl}-6-(4-methoxyphenyl)-1,3,5-triazine, as well as mixtures of the components named above. Further the addition of water-soluble UV filters is preferable. Preferred water soluble UV filters are 2-phenylbenzimidazole-5-sulfonic acid, phenylene-1,4-bis-(2-benzimidazyl)-3,3′-5,5′-tetrasulfonic acid and their alkali metal, alkaline earth metal, ammonium, alkylammonium, alkanolammonium and glucammonium salts, in particular the sulfonic acid itself with the INCI designation phenylbenzimidazole sulfonic acid (CAS.-No. 27503-81-7), which for example is available under the tradename Eusolex 232 from Merck or under Neo Heliopan Hydro from Symrise, and the phenylene-1,4-bis-(2-benzimidazyl)-3,3′-5,5′-tetrasulfonic acid disodium salt with the INCI designation disodium phenyl dibenzimidazole tetrasulfonate (CAS-No.: 180898-37-7), which is for example available under the tradename Neo Heliopan AP from Symrise, sulfonic acid derivatives of benzophenonene, preferably 2-hydroxy-4-methoxybenzophenone-5-sulfonic acid and its salts and sulfonic acid derivatives of the 3-benzylidenecamphor, such as e.g. 4-(2-oxo-3-bornylidenemethyl)benzene sulfonic acid.

Further particularly preferred sticks according to the invention, in particular deodorant or antiperspirant sticks, comprise the radical scavenger tris(tetra-methylhydroxy-piperidinol) citrate and the UV filter Bumetrizole for the purposes of product stabilization. Bumetrizole is preferably comprised in quantities of 0.01-0.1%, particularly preferred being 0.025-0.05 wt. %, relating to the total weight of the composition according to the invention.

Further particularly preferred sticks according to the invention, in particular deodorant or antiperspirant sticks, comprise at least one complexing substance for the purposes of product stabilization. Particularly preferred complexing substances are ethylenediaminetetraacetic acid (EDTA) and its sodium salts, such as are for example available under the tradename Trilon B from the firm BASF, further nitrilotriacetic acid (NTA) and its sodium salts, β-alanine diacetic acid and its salts and phosphonic acids and their salts. The complexing substance, at least one in number, is preferably comprised in a total weight of 0.01-0.5 wt. %, particularly preferred in a 0.08-0.2 wt. %, relating to the total weight of the composition according to the invention.

Further extraordinarily preferred sticks according to the invention, in particular deodorant or antiperspirant sticks, comprise at least one radical scavenger and at least one substance chosen from UV filters and complexants. Further extraordinarily preferred sticks according to the invention, in particular deodorant or antiperspirant sticks, comprise at least one radical scavenger, at least one UV filter and at least one complexant.

Hair Growth Inhibitors

Further particularly preferred sticks according to the invention, in particular deodorant or antiperspirant sticks, comprise at least one hair-growth inhibiting substance. Suitable substances that inhibit hair-growth are in particular chosen from eflornithine, substance combinations of soya protein hydrolysate, urea, menthol, salicylic acid and extracts of hypericum perforatum, hamamelis virginiana, arnica montana and the bark of Salix alba, such as is for example contained in the raw material “Pilinhib® Veg LS 9109” of Laboratoires Sérobiologiques with the INCI declaration “Propylene glycol, Hydrolyzed Soy Protein, Hypericum Perforatum Extract, Hamamelis Virginiana Extract, Arnica Montana Flower Extract, Urea, Salix Alba Bark Extract, Menthol, Salicylic acid,” further substance combinations of extracts of Epilobium angustifolium, the seeds of Cucurbita pepo (pumpkin) and the fruits of Serenoa serrulata, such as are for example and preferably contained in the raw materials “ARP 100” of Greentech S.A./Rahn with the INCI declaration “Water, Alcohol, Serenoa Serrulata Fruit Extract, Epilobium Angustifolium Extract, Cucurbita Pepo (Pumpkin) Seed Extract,” and “ARP 100 Huileux” (ex Greentech, INCI: Caprylic/Capric Triglyceride, Serenoa Serrulata Fruit Extract, Epilobium Angustifolium Flower/Leaf/Stem Extract, Cucurbita Pepo (Pumpkin) Seed Extract), further substance combinations of xylitol and the extracts of Citrus medica limonum (lemon) fruit, Carica papaya (papaya) and olive leaves, such as are contained for example and preferably in the raw material “Xyleine” from Impag/Seporga with the INCI declaration “Xylitol and Citrus Medica Limonum (Lemon) Fruit Extract and Carica Papaya (Papaya) Fruit Extract and Olea europaea (olive) leaf extract,” further substance combinations of Humulus lupulus, Viscum album, Salvia officinalis, Carica papaya and Thuya occidentalis, such as are contained for example and preferably in the raw material Plantafluid Complex AH of the firm Plantapharm with the INCI declaration “Aqua, Propylene Glycol, Humulus Lupulus, Viscum Album, Salvia Officinalis, Carica Papaya, Thuya Occidentalis,” as well as extracts of Larrea divaricata, such as are contained for example and preferably in the raw material Capislow from Sederma, which contains lecithin vesicles with a hydroglycolized extract of Larrea divaricata. Further preferred hair growth inhibitors are selected from the substances that inhibit the protein tyrosinkinase, in particular from Lavendustin-A, Erbstatin, Tyrphostin, Piceatannol, 4-hydroxybenzylidenemalononitrile, 3,5-di-tert-butyl-4-hydroxybenzylidenemalononitrile, α-cyano-(3,4-dihydroxy)-cinnamonitrile, α-cyano-(3,4,5-trihydroxy)cinnamonitrile, α-cyano-(3,4-dihydroxy)cinnamide, α-cyano-(3,4-dihydroxy)thiocinnamide, 2-amino-4-(4′-hydroxyphenyl)-1,1,3-tricyanobuta-1,3-diene, 2-amino-4-(3,4,5′-trihydroxyphenyl)-1,1,3-tricyanobuta-1,3-diene, 2-amino-4-(1H-alpha-indol-5-yl)-1,1,3-tricyanobuta-1,3-diene, 4-hydroxy-3-methoxy-5-(benzothiazolylthiomethyl)benzylidenecyanoacetamide, 4-amino-N-(2,5-dihydroxybenzyl)methyl benzoat, α-cyano-(3,4-dihydroxy)-cinnamonitrile, 4-(3-chloroanilino)-6,7-dimethoxyquinazoline, α-cyano-(3,4-dihydroxy)-N-benzylcinnamide, (−)-R—N-(α-methylbenzyl)-3,4-dihydroxybenzylidenecyanoacetamide, α-cyano-(3,4-dihydroxy)-N-(3-phenylpropyl)-cinnamide, α-cyano-(3,4-dihydroxy)-N-phenylcinnamide, α-cyano-(+)-(S)—N-(alpha-phenethyl)-(3,4-dihydroxy)cinnamide, α-cyano-(3,4-dihydroxy)-N-(phenylbutyl)cinnamide, Herbimycin A, thiazolidindione, phenazocin, 2,3-dihydro-2-thioxo-1H-indole-3-alkane acids, 2,2′-dithiobis-(1H-indole-3-alkane acids), sulfonylbenzoylnitrostyrene, methyl caffeate, HNMPA(AM)₃(hydroxy-2-naphthalenylmethylphosphonic acid tris-acetoxymethyl ester) and N-acetyl-Asp-Tyr-(2-malonyl)-Val-Pro-Met-Leu-NH₂. Further preferred hair growth inhibitors are selected from the substances disclosed in WO 2006/130330 A2, namely agonists of the farnesoid X-receptors, preferably selected from gallic acids, such as in particular lithocholic acid, cholic acid, deoxycholic acid, chenodeoxycholic acid, ursodeoxycholic acid and 6-alpha-ethylchenodeoxycholic acid; additionally from farnesoids, in particular farnesol (3,7,11-trimethyl-2,6,10-dodecatrien-1-ol), farnesal, farnesyl acetate, 3,7,11-trimethyl-2,6,10-dodecatriene-1-carboxylic acid, methyl farnesyl ether, methyl farnesoate, ethyl farnesyl ether, ethyl farnesoate; furthermore from 7-methyl-9-(3,3-dimethyloxivanyl)-3-methyl-2,6-nonadienoic acid methyl ester (Juvenile hormone III), 7-methyl-9-(3,3-dimethyloxivanyl)-3-methyl-2,6-nonadienoic acid ethyl ester, 3-alpha,7-alpha-dihydroxy-6-alpha-ethyl-5p-cholanic-24 acid, 7-alpha-dihydroxy-6-alpha-propyl-5p-cholanic-24 acid, 7-alpha-dihydroxy-6-alpha-allyl-5p-cholanic-24 acid, N-(2,2,2-trifluoroethyl)-N-[4-[2,2,2-trifluoro-1-hydroxy-1-(trifluoromethyl)-ethyl]phenyl]-benzene sulfoneanilide, 3-[2-[2-chloro-4-[3-(2,6-dichlorophenyl)-5-(1-methylethyl)-4-isoxazolyl]methoxy]-phenylethenyl]-benzoic acid, [3,5-bis(1,1-dimethylethyl)-4-hydroxyphenyl]ethenylidene]bisphosphonic acid tetraethyl ester, [2-[3,5-bis(1,1-dimethylethyl)-4-hydroxyphenyl]ethylidene]bisphosphonic acid tetrakis(1-methylethyl) ester, [2-[3,5-bis(1,1-dimethylethyl)-4-hydroxyphenyl]ethylidene]bisphosphonic acid tetraethyl ester and [3,5-bis(1,1-dimethylethyl-4-hydroxyphenyl]ethenylidene]bisphosphonic acid tetrakis(1-methylethyl)ester. The compositions according to the invention comprise at least one of the hair-growth inhibiting substances preferably in a quantity of 0.1-10 wt. %, preferably 0.5-5 wt. % and particularly preferably 1-4 wt. %, related respectively to the weight of the raw material tel quel and the total weight of the composition according to the invention.

Preservatives

Preferably, the usual preservatives can also be added to the compositions according to the invention, in order to prevent the decomposition of the product through microbial growth. Numerous preservatives also necessarily have deodorizing characteristics, so that some substances belong to both groups. Preferred preservatives for cosmetics are for example benzoic acid and its derivatives (e.g. propyl, phenyl and butyl benzoate, ammonium, sodium, potassium and magnesium benzoate), propionic acid and its derivatives (e.g. ammonium, sodium, potassium and magnesium propionate), salicylic acid and its derivatives (e.g. sodium, potassium and magnesium salicylate), 4-hydroxybenzoic acid and its esters and alkali-metal salts (e.g. methyl, ethyl, propyl, isopropyl, butyl, isobutyl, isodecyl, phenyl, phenoxyethyl and benzyl parabens, hexamidine parabens and di-parabens, sodium and potassium paraben, sodium and potassium methylparaben, potassium butylparaben, sodium and potassium propylparaben), alcohols and their salts (e.g. ethanol, propanol, isopropanol, benzyl alcohol, phenethyl alcohol, phenol, potassium phenolate, phenoxyethanol, phenoxyisopropanol, o-phenylphenol), guajacol and its derivatives, chlorhexidine and its derivatives (e.g. chlorhexidine diacetate, -digluconate, and -dihydrochloride), hydantoin and its derivatives (e.g. DEDM- and DMDM-hydantoin, DEDM-hydantoin dilaurate), urea and urea derivatives (e.g. diazolidinyl urea, imidazolidinyl urea), ferulaic acid and its derivatives (e.g. ethyl ferulate), sorbic acid and its derivatives (e.g. isopropyl sorbate, TEA sorbate, sodium, potassium and magnesium sorbate), isothiazole and oxazole derivatives (e.g. methylisothiazolinone, methylchloroisothiazolinone, dimethyloxazolidine), quaternary ammonium compounds (e.g. Polyquaternium-42, Quaternium-8, Quaternium-14, Quaternium-15), carbamates (e.g. iodopropynylbutyl carbamate), formaldehyde and sodium formate, glutaraldehyde, glyoxal, hexamidine, dehydracetic acid, 2-bromo-2-nitropropane-1,3-diol, isopropylcresol, methyldibromoglutaronitrile, polyaminopropylbiguanide, sodium hydroxymethyl glycinate, sodium phenol sulfonate, triclocarban, triclosan, zinc pyrithione, as well as diverse peptide antibiotics (e.g. Nisine). Preferred preservatives according to the invention are phenoxyethanol, the esters of 4-hydroxybenzoic acid, in particular methyl, ethyl, propyl, isopropyl, butyl and isobutyl paraben, as well as iodopropynylbutyl carbamate. The quantity of the preservative in the preferred compositions according to the invention is 0.001-10 wt. %, preferably 0.1-5 wt. % and in particular 0.1-3 wt. %, relating to the total weight of the combination.

In principle the subject of the present invention is to be extended to all cosmetic and dermatological stick compositions. Corresponding sticks for example can be mass-produced as lipsticks or concealer sticks and used through topical application on the skin.

Further preferred inventive stick compositions additionally comprise a cosmetic ingredient selected from monomers, oligomers and polymers of amino acids, N—C₂-C₂₄ acylamino acids, the esters and/or the physiologically compatible metal salts of these substances, DNA- or RNA-oligonucleotides, humidifiers, vitamins, provitamins and vitamin precursors of the groups A, B, C, E, H and K and the esters of the abovementioned substances, α-hydroxycarboxylic acids, α-ketocarboxylic acids, β-hydroxycarboxylic acids and esters, lactones or salt form thereof, flavonoids and flavonoid-rich vegetal extracts, isoflavonoids and isoflavonoid-rich vegetal extracts, polyphenols and polyphenol-rich vegetal extracts, ubiquinone and ubiquinol as well as their derivatives, silymarin, naturally occurring xanthine derivatives, selected from caffein, theophyllin, theobromine and aminophyllin, ectoin, inorganic and organic UV-filter substances, self-tanning actives, skin lighteners, skin calming actives, sebum regulators, antimicrobials, prebiotics as well as coloring, matting or lustrous pigments.

The monomers of the amino acids and/or of the N—C₂-C₂₄ acylamino acids are selected from alanine, arginine, aspartine, aspartic acid, canavanine, citrulline, cysteine, cystine, desmosine, dipalmitoylhydroxyproline, glutamine, glutamic acid, glycine, histidine, homophenylalanine, hydroxylysine, hydroxyproline, isodesmosine, isoleucine, leucine, lysine, methionine, methylnorleucine, ornithine, phenylalanine, proline, pyroglutamic acid, sarcosine, serine, taurine, threonine, thyroxine, tryptophan, tyrosine, vann, N-acetyl-L-cysteine, zinc pyroglutamate, sodium octanoyl glutamate, sodium decanoyl glutamate, sodium lauroyl glutamate, sodium myristoyl glutamate, sodium cetoyl glutamate and sodium stearoyl glutamate. Lysine, serine, N-acetyl-L-cysteine, zinc- and sodium pyroglutamate and sodium lauroyl glutamate are particularly preferred. The C₂-C₂₄ acyl group, with which the cited amino acids are derivatised on the amino group, is selected from an acetyl, propanoyl, butanoyl, pentanoyl, hexanoyl, heptanoyl, octanoyl, nonanoyl, decanoyl, undecanoyl, lauroyl, tridecanoyl, myristoyl, pentadecanoyl, cetoyl, palmitoyl, stearoyl, elaidoyl, arachidoyl or behenoyl group. Mixtures of C₈-C₁₈ acyl groups are also called cocoyl groups and are likewise preferred substituents. The amino acids that carry an OH group can also be esterified with the above-cited C₂-C₂₄ acyl groups on this OH group. An inventively preferred example of this is hydroxyproline that is N-acylated and esterified with two, preferably linear C₂-C₂₂ fatty acid groups, in particular dipalmitoyl hydroxyproline that is available from the Seppic Company under the name Sepilift PDHP. The physiologically compatible salts of the inventively preferred active substances that comprise acid groups and can form salts are selected from the ammonium, alkali metal, magnesium, calcium, aluminum, zinc and manganese salts. Sodium, potassium, magnesium, aluminum, zinc and manganese salts are preferred.

According to the invention, amino acid oligomers are understood to mean peptides containing 2-30, preferably 2-15, amino acids. The oligomers of the amino acids and/or of the N—C₂-C₂₄ acylamino acids are preferably selected from di-, tri-, tetra-, penta-, hexa- or pentadecapeptides, which can be N-acylated and/or esterified. Many of these amino acid oligomers stimulate the collagen synthesis or are capable of recruiting cells of the immune system, such as mast cells and macrophages, which then induce repair processes in the tissue by releasing growth factors, e.g. the collagen synthesis or are capable of binding onto the sequence Arg-Phe-Lys in Thrombospondin I (TSP-1) and thereby to release active TGF-β (tissue growth factor), which induces the synthesis of collagen in dermal fibroblasts. These types of amino acid oligomers can be used as active substances against skin aging. Inventively preferred, optionally N-acylated and/or esterified dipeptides are acetyl-citrullyl-arginine (e.g. Exsy-Algine from Exsymol with the INCI-Name Acetyl Citrull Amido Arginine), Tyr-Arg (Dipeptide-1), Val-Trp (Dipeptide-2), Asn-Phe, Asp-Phe, N-Palmitoyl-β-Ala-His, N-Acetyl-Tyr-Arg-hexyldecyl ester (e.g. Calmosensine from Sederma), Carnosine (β-Ala-His) and N-palmitoyl-Pro-Arg. Inventively preferred, optionally N-acylated and/or esterified tripeptides are Gly-His-Lys that e.g. is available under the name “Omega-CH-activator” from the GfN Company or in acylated form (N-palmitoyl-Gly-His-Lys) under the name Biopeptide CL from Sederma, but (in acylated form) also represents a constituent of the product Matrixyl 3000 from Sederma. The tripeptide Gly-His-Lys can also be employed as the copper salt (Cu2⁺) and as such is available from ProCyte Corporation. Moreover, analogs of Gly-His-Lys can be employed, wherein maximum two amino acids are substituted by other suitable amino acids. According to the invention, Ala, Leu and Ne are suitable for substituting Gly. The inventively preferred amino acids that can substitute His or Lys contain a side chain containing a nitrogen atom that is predominantly charged at pH 6, e.g. Pro, Lys, Arg, His, Desmosin and Isodesmosin. Lys is particularly preferably replaced by Arg, Orn or Citrullin. A further inventively preferred tripeptide is Gly-His-Arg (INCI Name: Tripeptide-3) as well as its derivative N-myristoyl-Gly-His-Arg, that e.g. is available under the name Collasyn 314-GR from Therapeutic Peptide Inc.; further inventively preferred tripeptides are selected from Lys-Val-Lys, Lys-Val-Dab (Dab=diamino butyric acid), Lys-Phe-Lys, Lys-Ile-Lys, Dab-Val-Lys, Lys-Val-Orn, Lys-Val-Dap (Dap=diamino propionic acid), Dap-Val-Lys, palmitoyl-Lys-Val-Lys, e.g. available from Pentapharm under the name SYN®-COLL, Lys-Pro-Val, Tyr-Tyr-Val, Tyr-Val-Tyr, Val-Tyr-Val (Tripeptide-2), Tripeptide-4 (e.g. ATPeptide, available from IMPAG), His-Ala-Orn N-elaidoyl-Lys-Phe-Lys and N-acetyl-Arg-Lys-Arg-NH₂. Inventively preferred, optionally N-acylated and/or esterified tetrapeptides are selected from rigin and rigin-based tetrapeptides as well as ALAMCAT tetrapeptides. Rigin has the sequence Gly-Gln-Pro-Arg. Rigin-based tetrapeptides include the rigin analogs and rigin derivatives, in particular the inventively particularly preferred N-palmitoyl-Gly-Gln-Pro-Arg that is available e.g. under the name Eyeliss from Sederma, but also represents a constituent of the product Matixyl 3000 from Sederma. The rigin analogs include those, in which the four amino acids are rearranged and/or in which at most two amino acids are substituted against rigin, e.g. the sequence Ala-Gln-Thr-Arg. At least one of the amino acids of the sequence preferably has a Pro or Arg and particularly preferably the tetrapeptide contains both Pro as well as Arg, wherein their sequence and position can vary. The substituting amino acids can be selected from each amino acids that is defined below. Particularly preferred rigin-based tetrapeptides include: Xaa-Xbb-Arg-Xcc, Xaa-Xbb-Xcc-Pro, Xaa-Xbb-Pro-Arg, Xaa-Xbb-Pro-Xcc, Xaa-Xbb-Xcc-Arg, wherein Xaa, Xbb and Xcc can be identical or different amino acids and wherein Xaa is selected from Gly and from the amino acids that can substitute Gly, Xbb is selected from Gln and from the amino acids that can substitute Gln, Xcc is selected from Pro or Arg and from amino acids that can substitute Pro and Arg. The preferred amino acids that can replace Gly contain an aliphatic side chain, e.g. β-Ala, Ala, Val, Leu, Pro, Sarcosine (Sar) and Isoleucine (Ne). The preferred amino acids that can replace Gln contain a side chain containing an amino group that is predominantly uncharged at neutral pH (pH 6-7), e.g. Asn, Lys, Orn, 5-hydroxyproline, citrulline and canavanine. The inventively preferred amino acids that can substitute Arg contain a side chain containing a nitrogen atom that is predominantly charged at pH 6, e.g. Pro, Lys, His, Desmosin and Isodesmosin. According to the invention, Gly-Gln-Arg-Pro and Val-Val-Arg-Pro are preferred as rigin analogs. ALAMCAT tetrapeptides are tetrapeptides that contain at least one amino acid having an aliphatic side chain, e.g., β-Ala, Alan, Val, Leu, Pro, sarcosine (Sar), and isoleucine (Ile). ALAMCAT tetrapeptides furthermore contain at least one amino acid having a side chain with an amino group that is present in predominantly uncharged fashion at neutral pH (pH 6-7), e.g., Gln, Asn, Lys, Orn, 5-hydroxyproline, citrulline, and canavanine. ALAMCAT tetrapeptides furthermore contain at least one amino acid having a side chain with a nitrogen atom that is present in predominantly charged state at pH 6, e.g., Arg, Pro, Lys, His, desmosine, and isodesmosine. ALAMCAT tetrapeptides can contain any desired amino acid as a fourth amino acid; preferably, however, the fourth amino acid is also selected from the three groups cited above.

Optionally N-acylated and/or esterified pentapeptides preferred according to the present invention are selected from Lys-Thr-Thr-Lys-Ser and its N-acylated derivatives, particularly preferably N-palmitoyl-Lys-Thr-Thr-Lys-Ser, which is obtainable from the Sederma company under the designation Matrixyl, furthermore N-palmitoyl-Tyr-Gly-Gly-Phe-Met, Val-Val-Arg-Pro-Pro, N-palmitoyl-Tyr-Gly-Gly-Phe-Leu, Gly-Pro-Phe-Pro-Leu, and N-benzyloxycarbonyl-Gly-Pro-Phe-Pro-Leu (the latter two represent serine proteinase inhibitors to inhibit desquamation). Optionally N-acylated and/or esterified hexapeptides preferred according to the present invention are Val-Gly-Val-Ala-Pro-Gly and its N-acylated derivatives, particularly preferably N-palmitoyl-Val-Gly-Val-Ala-Pro-Gly, which is obtainable from the Sederma company under the designation Biopeptide EL, furthermore Acetyl Hexapeptide-3 (Argireline of Lipotec), Hexapeptide-4 (e.g., Collasyn 6KS of Therapeutic Peptide Inc. (TPI)), Hexapeptide-5 (e.g., Collasyn 6VY of TPI), Myristoyl Hexapeptide-5 (e.g., Collasyn 614VY of TPI), Myristoyl Hexapeptide-6 (e.g., Collasyn 614VG of TPI), Hexapeptide-8 (e.g., Collasyn 6KS of TPI), Myristoyl Hexapeptide-8 (e.g., Collasyn Lipo-6KS of TPI), Hexapeptide-9 (e.g., Collaxyl of Vincience), and Hexapeptide-10 (e.g., Collaxyl of Vincience or Seriseline of Lipotec), Ala-Arg-His-Leu-Phe-Trp (Hexapeptide-1), Acetyl Hexapeptide-1 (e.g., Modulene of Vincience), Acetyl Glutamyl Hexapeptide-1 (e.g., SNAP-7 of Centerchem), Hexapeptide-2 (e.g., Melanostatine-DM of Vincience), Ala-Asp-Leu-Lys-Pro-Thr (Hexapeptide-3, e.g., Peptide 02 of Vincience), Val-Val-Arg-Pro-Pro-Pro, Hexapeptide-4 (e.g., Collasyn 6KS of Therapeutic Peptide Inc. (TPI)), Hexapeptide-5 (e.g., Collasyn 6VY of TPI), Myristoyl Hexapeptide-5 (e.g., Collasyn 614VY of TPI), Myristoyl Hexapeptide-6 (e.g., Collasyn 614VG of TPI), Ala-Arg-His-methylnorleucine-homophenylalanine-Trp (Hexapeptide-7), Hexapeptide-8 (e.g., Collasyn 6KS of TPI), Myristoyl Hexapeptide-8 (e.g., Collasyn Lipo-6KS of TPI), Hexapeptide-9 (e.g., Collaxyl of Vincience), Hexapeptide-10 (e.g., Collaxyl of Vincience or Seriseline of Lipotec) and Hexapeptide-11 (e.g., Peptamide-6 of Arch Personal Care). A pentadecapeptide preferred according to the present invention is, for example, the raw material Vinci 01 of Vincience (Pentadecapeptide-1). A further preferred amino acid oligomer is the peptide derivative L-glutamylaminoethyl indole (Glistin of Exsymol). Particularly preferred according to the present invention is the combination of N-palmitoyl-Gly-His-Lys and N-palmitoyl-Gly-Gln-Pro-Arg, available, for example, in the raw material Matrixyl 3000 of the Sederma Company.

The polymers of the amino acids and/or of the N—C₂-C₂₄ acylamino acids are preferably selected from vegetal and animal protein hydrolysates and/or proteins containing more than 30 amino acid units. Animal protein hydrolysates are, for example, elastin, collagen, keratin, silk, conchioline and milk protein hydrolysates, which can also be present in the form of salts. According to the invention, protein hydrolyzates of vegetal origin, e.g. soya, wheat, almond, pea, potato and rice protein hydrolyzates, are preferred. Corresponding commercial products are e.g. DiaMin® (Diamalt), Gluadin® (Cognis), Lexein® (Inolex) and Crotein® (Croda). Soya protein hydrolyzates are particularly preferred with an average molecular weight in the range 1200-1800 Dalton, preferably in the range 1400-1700 Dalton, e.g. under the trade name Ridulisse C® from the Silab Company, and soya protein hydrolyzates with an average molecular weight in the range 600-1000 Dalton, preferably 800 Dalton, available under the tradename Phytokine® from Coletica, soya protein hydrolyzates that are N-acylated and/or esterified with coco fatty acids in the form of their alkali metal salts. Coco fatty acids principally include alkane carboxylic acids containing 8-18 carbon atoms, in particular caprylic acid, capric acid, lauric acid, myristic acid, palmitic acid and stearic acid. Preferred alkali metal salts are selected from lithium, sodium and potassium salts, wherein potassium salts are particularly preferred. Another inventively particularly preferred soya protein hydrolyzate is a soya protein hydrolyzate that is N-acylated and/or esterified with coco fatty acid in the form of the potassium salt, available under the tradename Cococopolipeptide di Soja from the Sinerga Company. Keratin hydrolyzates are also inventively preferred, in particular wool keratin hydrolyzates. A particularly preferred wool keratin hydrolyzate is available under the tradename Keratec Pep from Croda. Keratic Pep has a low molecular weight fraction with an average molecular weight of 150 Dalton and a higher molecular weight fraction with an average molecular weight of 1265 Dalton. Conchiolin hydrolyzates are also inventively preferred, in particular those that are available under the tradenames Pearl Protein Extract and Pearl Protein Extract BG from Maruzen. Conchiolin is a complex protein that is produced from the external epithelium of molluscs, in particular from pearl mussels and various types of snail and which forms the very stable shell of these molluscs by storing calcium carbonate crystals. Protein hydrolyzates can also naturally comprise monomeric amino acids and oligopeptides; their composition is normally not defined. Likewise, it is possible to employ acyl derivatives of the protein hydrolyzates, e.g. in the form of their fatty acid condensation products. The corresponding commercial products are e.g. Lamepon® (Cognis), Gluadin® (Cognis), Lexein® (Inolex), Crolastin® or Crotein® (Croda).

Cationized protein hydrolyzates are also preferred according to the invention. Cationic protein hydrolyzates are particularly preferred, whose base protein content has a molecular weight of 100 to 25 000 Daltons, preferably 250 to 5 000 Daltons. Moreover, cationic protein hydrolyzates are understood to include quaternized amino acids and their mixtures. Moreover, the cationic protein hydrolyzates can also be further derivatized. Some of the products listed under INCI names in the “International Cosmetic Ingredient Dictionary and Handbook,” (seventh edition 1997, The Cosmetic, Toiletry, and Fragrance Association, Washington, D.C.) and available commercially may be listed as typical examples of the cationic protein hydrolysates and derivatives used according to the present invention: Cocodimonium Hydroxypropyl Hydrolyzed Collagen, Steardimonium Hydroxypropyl Hydrolyzed Collagen, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl. The cationic protein hydrolyzates and derivatives based on plants are quite particularly preferred.

In a further preferred embodiment, the polymers of the amino acids comprised in the stick compositions according to the invention are selected from DNA repair enzymes. DNA repair enzymes preferred according to the invention are photolyase and T4 endonuclease V, the latter hereinafter abbreviated to “T4N5”. These two enzymes are already known in the existing art as DNA repair enzymes. “DNA repair” is to be understood, by definition, as the cleavage or removal of UV-induced pyrimidine dimers from DNA. “Photolyase” is the abbreviation for deoxyribopyrimidine photolyase or DNA photolyase, an enzyme having the classification number EC 4.1.99.3. A particularly efficient photolyase stems from Anacystis nidulans, a phototrophic marine microorganism. The photolyase from A. nidulans is now obtained in industrially relevant quantities from E. coli. Photolyase is dependent on light for activation. The enzyme T4 Endonuclease V is produced by the denV gene of the T4 bacteriophage, and is one of the phosphodiesterases that hydrolytically cleave nucleic acids at the (5′-3′) bond. T4N5 is active even without the influence of light. The use of liposome-encapsulated DNA repair enzymes is particularly preferred according to the invention. Liposome-encapsulated photolyase is obtainable commercially, for example, under the product designation Photosome™, and liposome-encapsulated T4N5, for example, under the designation Ultrasome™, from the AGI Dermatics Company, USA. Particularly preferred inventive stick compositions comprise at least one of the commercial products Photosomes™ or Ultrasomes™ in total quantities of 0.1 to 10 wt. %, preferably 0.5 to 5.0 wt. % and particularly preferably 1.0 to 4.0 wt. %, based on the total stick composition according to the invention. Particularly preferred inventive stick compositions comprise at least one monomer, oligomer or polymer of amino acids, N—C₂-C₂₄ acylamino acids and/or the esters and/or the physiologically compatible metal salts of these substances in total quantities of 0.0000001-10 wt. %, preferably 0.001 to 5 wt. % and particularly preferably 0.01-1-2-3 wt. %, each based on the active substance content in the total stick composition according to the invention.

In a further preferred embodiment, the stick compositions according to the invention comprise at least one DNA oligonucleotide or at least one RNA oligonucleotide. According to the invention, an “oligonucleotide” is understood to mean polymerizates of 2 to 20, preferably 2 to 10 mononucleotides that, as in the case of polynucleotides and nucleic acids, are linked by phosphoric acid diester bridges. The nucleotides are made up of nucleobases (usually derivatives of pyrimidine or purine), pentoses (mostly D-ribofuranose or 2-deoxy-D-ribofuranose in a β-N-glycoside bond onto the nucleobase) and phosphoric acid. The mononucleotides are, for example, adenosine phosphates, cytidine phosphates, guanosine phosphates, uridine phosphates and thymidine phosphates, in particular CMP (cytidine 5′-monophosphate), UDP (uridine 5′-diphosphate), ATP (adenosine 5′-triphosphate), and GTP (guanosine 5′-triphosphate). An oligonucleotide that is particularly preferred according to the invention is the thymidine dinucleotide. Particularly preferred inventive stick compositions comprise at least one DNA oligonucleotide and/or one RNA oligonucleotide in total quantities of 0.0000001 to 5 wt. %, preferably 0.0001 to 0.5 wt. % and particularly preferably 0.001 to 0.05 wt. %, based on the total composition.

In a further preferred embodiment, the stick compositions according to the invention comprise at least one natural betaine compound. Natural betaine compounds that are preferred according to the invention are naturally occurring compounds having the atomic grouping R₃N⁺—CH₂—X—COO⁻ according to IUPAC Rule C-816.1. Betaine surfactants (synthetic) are not included among the betaine compounds used according to the invention; nor are other zwitterionic compounds in which the positive charge is located on N or P and the negative charge formally on O, S, B, or C, but that do not correspond to IUPAC Rule C-816.1. Betaine compounds preferred according to the invention are betaine (Me₃N⁺—CH₂—COO⁻) and carnitine (Me₃N⁺—CH₂—CHOH—CH₂—COO⁻), each with Me=methyl and X═C—C single bond (in the case of betaine) or X=—CHOH—CH₂— for the case of carnitine. Particularly preferred inventive stick compositions according to the invention comprise at least one natural betaine compound in total quantities of 0.05 to 5 wt. %, preferably 0.1 to 3 wt. % and particularly preferably 0.5 to 2 wt. %, each based on the total stick composition.

In a further preferred embodiment, the stick compositions according to the present invention comprise at least one vitamin, provitamin, or a compound designated as a vitamin precursor, from the vitamin groups A, B, C, E, H, and K and the esters of the aforementioned substances.

The group of substances designated as vitamin A includes retinol (vitamin A₁) as well as 3,4-didehydroretinol (vitamin A₂). β-Carotene is the provitamin of retinol. Examples of particularly preferred vitamin A components according to the invention are vitamin A acid and its esters, vitamin A aldehyde and vitamin A alcohol as well as its esters, such as retinyl palmitate and retinyl acetate. Particularly preferred compositions according to the invention comprise, in addition to the at least one alkyl or hydroxyalkyl substituted urea of Formula (A), in particular (2-hydroxyethyl)urea, at least one vitamin, provitamin or a compound designated as a vitamin precursor of the vitamin group A or at least one ester thereof in total quantities of 0.001-2 wt. %, preferably 0.5-0.5-1 wt. %, based on the total composition.

The vitamin B group or the vitamin B complex include, inter alia
vitamin B₁, trivial name thiamine, chemical name 3-[(4′-amino-2′-methyl-5′-pyrimidinyl)-methyl]-5-(2-hydroxyethyl)-4-methylthiazolium chloride. Thiamine hydrochloride is preferably added in amounts of 0.0005 to 0.1-1 wt. %, based on the total composition according to the invention.
vitamin B₂, trivial name riboflavin, chemical name 7,8-dimethyl-10-(1-D-ribityl)-benzo[g]pteridine-2,4(3H,10H)-dione. Riboflavin or its derivatives are preferably added in amounts of 0.0005 to 0.1-1 wt. %, based on the total composition according to the invention.
vitamin B₃. The compounds nicotinic acid and nicotinamide (niacinamide) are included under this designation. According to the invention, nicotinamide is preferred and is preferably comprised in the compositions according to the invention in amounts of 0.0005 to 0.1-1 wt. %, based on the total composition according to the invention.
vitamin B₅ (pantothenic acid and panthenol). Preferably, panthenol is added. Preferred derivatives of panthenol according to the invention are especially the esters and ethers of panthenol as well as cationically derivatized panthenols. In a further preferred embodiment of the invention, derivatives of 2-furanone with the general structural formula (VIT-I) are also added instead of, or in addition to, pantothenic acid or panthenol.

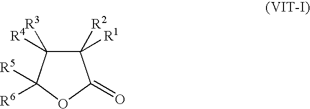

Particularly preferred 2-furanone derivatives are those in which the substituents R¹ to R⁶, independently of each other, represent a hydrogen atom, a hydroxyl group, a methyl, methoxy, aminomethyl or hydroxymethyl group, a saturated or singly or doubly unsaturated, linear or branched C₂-C₄ hydrocarbon group, a saturated or singly or doubly unsaturated, linear or branched mono-, di- or trihydroxy C₂-C₄ hydrocarbon group or a saturated or singly or doubly unsaturated, linear or branched mono-, di- or triamino C₂-C₄ hydrocarbon group. Particularly preferred derivatives are also the commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), wherein all stereoisomers are expressly included. According to the invention, the greatly preferred 2-furanone derivative is pantolactone (dihydro-3-hydroxy-4,4-dimethyl-2(3H)-furanone), wherein in Formula (VIT-I) R¹ stands for a hydroxy group, R² for a hydrogen atom, R³ and R⁴ for a methyl group and R⁵ and R⁶ for a hydrogen atom. The stereoisomer (R)-pantolactone results from the degradation of pantothenic acid. Particularly preferred inventive compositions according to the invention comprise at least one of the cited compounds of the vitamin B₅ type as well as the 2-furanon derivatives in a total quantity of 0.05 to 5 wt. %, preferably 0.1 to 3 wt. %, particularly preferably 0.5 to 2 wt. %, each based on the total composition.
Vitamin B₆, understood not to mean a pure substance, but rather the known derivatives of 5-hydroxymethyl-2-methylpyridin-3-ol with the trivial names pyridoxine, pyridoxamine and pyridoxal. Particularly preferred compositions according to the invention comprise at least one vitamin B₆ component in a total quantity of 0.0001 to 1.0 wt. %, particularly in quantities of 0.001 to 0.01 wt. %.
Vitamin B₇ (biotin), also designated as Vitamin H or “skin vitamin”. Biotin is (3aS,4S,6aR)-2-oxohexahydrothienol[3,4-d]-imidazole-4-valeric acid. Particularly preferred compositions according to the invention comprise at least one component selected from biotin and the biotin esters in a total quantity of 0.0001 to 1.0 wt. %, particularly 0.001 to 0.01 wt. %.
Folic acid (vitamin B₉, vitamin B_{c}). The international non-proprietary name for N-[4-(2-amino-3,4-dihydro-4-oxo-6-pteridinylmethylamino)-benzoyl]-L-glutamic acid (N-Pteroyl-L-glutamic acid, PteGlu). Folate is used synonymously with pteroyl glutamate; folates is the collective term for all folic acid active compounds, and designates a substance class that contains a pteridine ring joined to 4-amino benzoic acid and L-glutamic acid. Folic acid is a growth factor for various microorganisms and a compound having vitamin characteristics, which occurs in nature usually as a polyglutamate and in reduced form (7,8-dihydrofolic acid, H₂folate, DHF; tetrahydrofolic acid, H₄folate, THF; 5′-methyltetrahydrofolic acid, CH₃—H₄folate, MeTHF).
Compositions particularly preferred according to the invention comprise at least one component selected from folic acid, folates, and esters thereof, in a total quantity from 0.0001 to 1.0 wt %, in particular 0.01 to 0.5 wt %, based on the composition.
Orotic acid (vitamin B₁₃, 1,2,3,6-tetrahydro-2,6-dioxo-4-pyrimidinecarboxylic acid, uracil-6-carboxylic acid, whey acid). Orotic acid, its choline esters, or orotic acid metal salts (orotates of Ca, Cr, Fe, K, Co, Cu, Li, Mg, Mn, Na, Zn, Sn), are particularly preferred according to the invention. Compositions particularly preferred according to the invention comprise at least one component selected from orotic acid, orotates, and esters thereof, in a total quantity from 0.0001 to 1.0 wt %, in particular 0.01 to 0.5 wt %, based on the composition.

In a further preferred embodiment, the stick compositions according to the invention comprise at least one substance selected from the vitamins, provitamins and vitamin precursors of the group B₁, B₂, B₃, B₆, B₇ and their esters and pantolactone.

Preferred vitamins, provitamins and vitamin precursors of the C group and their esters are vitamin C (ascorbic acid) and the derivatives ascorbyl palmitate, ascorbyl stearate, ascorbyl dipalmitate, ascorbyl acetate, magnesium ascorbyl phosphate, sodium ascorbyl phosphate, sodium and magnesium ascorbate, disodium ascorbyl phosphate and sulfate, potassium ascorbyl tocopheryl phosphate, chitosan ascorbate or ascorbyl glucoside. The combination with tocopherols can also be preferred. Particularly preferred compositions according to the invention comprise at least one of the cited compounds of the vitamin C type in a total quantity of 0.05 to 5 wt. %, preferably 0.1 to 3 wt. %, particularly preferably 0.5 to 1-2 wt. %, each based on the total composition. The vitamin E group includes tocopherol, in particular α-tocopherol, and its derivatives. Preferred derivatives are in particular the esters, such as tocopheryl acetate, tocopheryl nicotinate, tocopheryl phosphate, tocopheryl succinate, tocopheryl linoleate, tocopheryl oleate, tocophereth-5, tocophereth-10, tocophereth-12, tocophereth-18, tocophereth-50, and tocophersolan. Particularly preferred compositions according to the invention comprise at least one substance selected from tocopherol and its derivatives in a total quantity of 0.05 to 5 wt. %, preferably 0.1 to 3 wt. %, particularly preferably 0.5 to 1-2 wt. %, each based on the total composition. Vitamin H is another term for biotin or vitamin B₇ (see above). Among the fat-soluble vitamins of the vitamin K group, based on the fundamental structure of 2-methyl-1,4-naphthoquinone, are phylloquinone (vitamin K₁), farnoquinone or menaquinone-7 (vitamin K₂) and menadione (vitamin K₃). Particularly preferred compositions according to the invention comprise at least one vitamin K in a total quantity of 0.0001 to 1 wt. %, preferably 0.05 to 0.01 wt. %, particularly preferably 0.1 to 0.5 wt. %, each based on the total composition.

Vitamin A palmitate (retinyl palmitate), pantolactone, nicotinic acid amide, pyridoxine, pyridoxamine, pyridoxal, biotin, ascorbyl palmitate and acetate, Mg ascorbyl phosphate, Na ascorbyl phosphate, sodium and magnesium ascorbate, and the tocopherol esters, especially tocopheryl acetate, are particularly preferred according to the invention.

In a further preferred embodiment, the stick compositions according to the invention comprise at least one α-hydroxycarboxylic acid, α-ketocarboxylic acid or β-hydroxycarboxylic acid or their ester, lactone or salt form. Inventively preferred α-hydroxycarboxylic acids or α-ketocarboxylic acids are glycolic acid, lactic acid, tartaric acid, citric acid, 2-hydroxybutanoic acid, 2,3-dihydroxypropanoic acid, 2-hydroxypentanoic acid, 2-hydroxyhexanoic acid, 2-hydroxyheptanoic acid, 2-hydroxyoctanoic acid, 2-hydroxydecanoic acid, 2-hydroxydodecanoic acid, 2-hydroxytetradecanoic acid, 2-hydroxyhexadecanoic acid, 2-hydroxyoctadecanoic acid, mandelic acid, 4-hydroxymandelic acid, malic acid, meso-tartaric acid, glucaric acid, galactaric acid, aldaric acid, gularic acid, 2-hydroxy-2-methylsuccinic acid, gluconic acid, pyruvic acid, glucuronic acid and galacturonic acid. Particularly preferred α-hydroxycarboxylic acids are lactic acid, citric acid, glycolic acid and gluconic acid. A particularly preferred β-hydroxycarboxylic acid is salicylic acid. The esters of the cited acids are selected from the methyl, ethyl, propyl, isopropyl, butyl, amyl, pentyl, hexyl, 2-ethylhexyl, octyl, decyl, dodecyl and hexadecyl esters. Particularly preferred inventive stick compositions comprise at least one α-hydroxycarboxylic acid, α-ketocarboxylic acid or β-hydroxycarboxylic acid or their ester, lactone or salt form in a total quantity of 0.01 to 10 wt. %, preferably 0.1 to 5 wt. % and particularly preferably 0.5 to 1-2 wt. %, based on the total composition.

In a further preferred embodiment, the compositions according to the invention comprise at least one flavonoid or at least one flavonoid-rich plant extract.

The flavonoids preferred according to the present invention encompass the glycosides of the flavones, of the flavanones, of 3-hydroxyflavone (flavonols), of the aurones, and of the isoflavones. Particularly preferred flavonoids are selected from naringin (aurantiin, naringenin-7-rhamnoglucoside), α-glucosyl rutin, α-glucosyl myricetin, α-glucosyl isoquercetin, α-glucosyl quercetin, dihydroquercetin (taxifolin), hesperidin (3′,5,7-trihydroxy-4′-methoxyflavanon-7-rhamnoglucoside, hesperitin-7-O-rhamnoglucoside), neohesperidin, rutin (3,3′,4′,5,7-pentahydroxyflavone-3-rhamnoglucoside, quercetin-3-rhamnoglucoside), troxerutin (3,5-dihydroxy-3′,4′, 7-tris(2-hydroxyethoxy)-flavone-3-(6-O-(6-deoxy-α-L-mannopyranosyl)-β-D-glucopyranoside)), monoxerutin (3,3′,4′,5-tetrahydroxy-7-(2-hydroxyethoxy)-flavone-3-(6-O-(6-deoxy-α-L-mannopyranosyl)-β-D-glucopyranoside)), diosmin (3′,4′,7-trihydroxy-5-methoxyflavanone-7-rhamnoglucoside), eriodictin and apigenin-7-glucoside (4′,5,7-trihydroxyflavone-7-glucoside). Inventively extremely preferred flavonoids are α-glucosylrutin, naringin and apigenin-7-glucoside. Also preferred are the biflavonoids constructed from two flavonoid units, which occur e.g., in ginkgo species. Further preferred flavonoids are the chalcones, principally phloricin, hesperidin methylchalcone and neohesperidin dihydrochalcone. Particularly preferred compositions according to the invention comprise at least one flavonoid in a total quantity of 0.0001 to 1 wt. %, preferably 0.0005 to 0.5 wt. % and particularly preferably 0.001 to 0.1 wt. %, each based on the flavonoid active substance in the total cosmetic composition.

In a further preferred embodiment, the compositions according to the invention comprise at least one isoflavonoid or at least one isoflavonoid-rich plant extract. Included among the isoflavonoids at this juncture are the isoflavones and the isoflavone glycosides. In the context of the present invention, isoflavones are understood to be substances that represent the hydrogenation, oxidation, or substitution products of 3-phenyl-4H-1-benzopyran; a hydrogenation can be present at the 2,3-position of the carbon structure, and oxidation can be present to form a carbonyl group in the 4-position; “substitution” is to be understood as the replacement of one or more hydrogen atoms by hydroxy or methoxy groups. Among the isoflavones preferred according to the invention are, for example, daidzein, genistein, prunetin, biochanin, orobol, santal, pratensein, irigenin, glycitein, biochanin A and formononetin. Daidzein, genistein, glycitein, and formononetin are particularly preferred as isoflavones. In the isoflavone glycosides preferred according to the invention, the isoflavones are glycosidically linked via at least one hydroxyl group to at least one sugar. Suitable sugars are mono- or oligosaccharides, in particular D-glucose, D-galactose, D-glucuronic acid, D-galacturonic acid, D-xylose, D-apiose, L-rhamnose, L-arabinose and rutinose. Daidzin and genistin are particularly preferred isoflavone glycosides according to the invention. It is further preferred according to the invention if the isoflavones and/or glycosides thereof are contained in the preparations as constituents of a substance mixture obtained from a plant, in particular of a plant extract. Plant-based substance mixtures of this kind can be obtained, in the manner commonly known to one skilled in the art, for example by being extracted or pressed out from plants such as soy, in particular from soybeans, red clover, or chickpeas. Particularly preferably, isoflavones or isoflavone glycosides are used in the preparations according to the invention in the form of extracts obtained from soy, such as those commercially obtainable, for example, under the product designation Soy Protein Isolate SPI (Protein Technology International, St. Louis) or Soy Phytochemicals Concentrate SPC (Archer Daniels Midland, Decatur). A further particularly preferred isoflavonoid-rich plant extract is apple-core extract, in particular the commercial product Ederline of Seporga. Ederline contains phytohormones, isoflavonoids, phytosterols, triterpenoids, tocopherol and natural waxes. Particularly preferred compositions according to the invention comprise at least one isoflavonoid in a total quantity of 0.00001 to 1 wt. %, preferably 0.0005 to 0.5 wt. % and particularly preferably 0.001 to 0.1 wt. %, each based on the isoflavonoid active substance in the total cosmetic composition.

In a further preferred embodiment, the compositions according to the invention comprise at least one polyphenol or a polyphenol-rich plant extract. According to the invention, polyphenols are understood to include aromatic compounds that comprise at least two phenolic hydroxyl groups in the molecule. These include the three dihydroxybenzenes catechol, resorcinol, and hydroquinone, furthermore phloroglucin, pyrogallol, and hexahydroxybenzene. In nature, free and etherified polyphenols occur, for example, in blossom dyes (anthocyanidines, flavones), in tanning agents (catechins, tannins), as lichen or fern ingredients (usninic acid, acylpolyphenols), in lignins and as gallic acid derivatives. Preferred polyphenols are flavones, catechins, usninic acid and, as tannins, the derivatives of gallic acid, digallic acid, and digalloylgallic acid. Particularly preferred polyphenols are the monomeric catechines, i.e., the derivatives of the flavan-3-ols, and leukoanthocyanidines, i.e., the derivatives of the leucoanthocyanidines that carry phenolic hydroxyl groups preferably in the 5,7,3′,4′,5′-position, preferably epicatechin and epigallocatechin, as well as the tanning agents resulting there from by autocondensation. Tanning agents of this kind are preferably used not as an isolated pure substance but as extracts of plant parts that are rich in tanning agents, e.g., extracts of catechu, quebracho, oak bark and pine bark as well as other tree barks, leaves of green tea (Camellia sinensis), and mate. The tannins are likewise particularly preferred. A particularly preferred polyphenol-rich cosmetic active substance is the commercial product Sepivinol R, an extract from red wine, obtainable from the Seppic company. A further particularly preferred polyphenol-rich cosmetic active substance is the commercial product Crodarom Chardonnay L, an extract from the seeds of the Chardonnay grape, obtainable from the Croda company. According to the invention, the polyphenols are preferably employed in amounts of 0.001 to 10 wt %, particularly preferably 0.005 to 5 wt %, and extremely preferably 0.01 to 3 wt %, based in each case on the weight of the commercial product that comprises at least one polyphenol, in the total inventive composition.

In a further preferred embodiment, the compositions according to the invention comprise at least one ubiquinone, an ubiquinol or their derivatives. Ubiquinols are the reduced form of the ubiquinones. The inventively preferred ubiquinones have the following Formula (UBI-I):

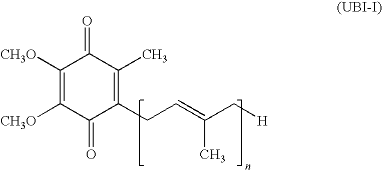

with n=6, 7, 8, 9 or 10.
The ubiquinone of the Formula (UBI-I) with n=10, also known as the coenzyme Q10, is particularly preferred. Particularly preferred compositions according to the invention comprise at least one ubiquinone, ubiquinol or a derivative thereof in a total quantity of 0.0001 to 1 wt. %, preferably 0.001 to 0.5 wt. % and particularly preferably 0.005 to 0.1 wt. %, each based on the total composition.

In a further preferred embodiment, the inventive compositions comprise silymarin. Silymarin represents, according to the invention, an active substance concentrate, previously considered a uniform substance, from the fruits of the milk thistle (Silybum marianum). The principal constituents of silymarin are silybin (silymarin I), silychristin (silymarin II), and silydianin, which belong to the group of the flavanolignans. Particularly preferred compositions according to the invention comprise silymarin in quantities of 0.00001 to 1 wt. %, preferably 0.0001 to 0.01 wt. % and particularly preferably 0.005 to 0.1 wt. %, each based on the total composition. In a further preferred embodiment, the compositions according to the present invention comprise at least one xanthine derivative of natural origin, selected from caffeine, theophylline, theobromine and aminophylline. Particularly preferred compositions according to the invention comprise xanthine derivatives in quantities of 0.0001 to 1 wt. %, preferably 0.001 to 0.5 wt. % and particularly preferably 0.005 to 0.1 wt. %, each based on the total composition. In a further preferred embodiment, the inventive compositions comprise ectoin. Ectoin is the trivial name for 2-methyl-1,4,5,6-tetrahydropyrimidine-4-carboxylate. Particularly preferred compositions according to the invention comprise ectoin in quantities of 0.001 to 0.5 wt. % and particularly preferably 0.005 to 0.01 wt. %, each based on the total composition.

In a further preferred embodiment, the inventive compositions comprise creatine. Creatine is the trivial name for N-methylguanidinoacetic acid or N-amidinosarcosin. Preferred compositions according to the invention comprise creatine in quantities of 0.001 to 0.5 wt. % and particularly preferably 0.01 to 0.1 wt. %, each based on the total composition.

In a further preferred embodiment, the compositions according to the invention comprise at least one olive leaf extract (Olea Europaea (olive) leaf extract). An inventively particularly preferred olive leaf extract is available under the tradename Oleanoline DPG from the Vincience company. Another inventively particularly preferred olive leaf extract is available under the tradename Olea europ Fol extr. S. sicc. From the Fruitarom company. Particularly preferred compositions according to the invention comprise at least one olive leaf extract in a total quantity of 0.01 to 5 wt. %, preferably 0.1 to 3 wt. % and particularly preferably 0.5 to 1-2 wt. %, each based on the extract as the commercial product tel quel in the total composition according to the invention.

Olive leaf extracts can possess a high content of oleanolic acid and/or oleanol. In a further preferred embodiment, the inventive compositions comprise oleanolic acid and/or oleanol. Particularly preferred compositions according to the invention comprise oleanolic acid and/or oleanol in a total quantity of 0.00001 to 2 wt. %, preferably 0.001 to 1 wt. % and particularly preferably 0.05 to 0.1 wt. %, each based on the total composition according to the invention.

In a further preferred embodiment, the inventive compositions comprise ursolic acid. Particularly preferred compositions according to the invention comprise ursolic acid in a total quantity of 0.00001 to 2 wt. %, preferably 0.001 to 1 wt. % and particularly preferably 0.05 to 0.1 wt. %, each based on the total composition according to the invention.

In a further preferred embodiment, the inventive compositions comprise at least one active substance selected from the mono- and polyhydroxystilbenes and their esters. According to the invention, polyhydroxystilbenes are understood to be stilbenes that are substituted with 2, 3, 4, 5, 6, 7, 8, 9 or 10 hydroxyl groups on both phenyl moieties, wherein said groups can be esterified. Mono- and polyhydroxystilbenes and their esters increase and/or improve the interaction between the extra cellular matrix and the fibroblasts. Inventively particularly preferred hydroxystilbenes and their esters are selected from resveratrol (trans-stilbene-3,4′,5-triol), the resveratrol mono-, -di- and -triphosphoric acid esters and their salts, as well as from hydroxystilbene oligomers, e.g. epsilon-viniferin. An inventively particularly preferred resveratrol ester of phosphoric acid is trisodium resveratrol triphosphate, available from e.g. Ajinomoto.

Particularly preferred cosmetic or dermatological compositions according to the invention comprise at least one active substance selected from the mono- and polyhydroxystilbenes and esters thereof in a total quantity of 0.000001 to 5 wt. %, preferably 0.00001 to 1 wt. %, particularly preferably 0.0001 to 0.1 wt. % and extremely preferably 0.005 to 0.05 wt. %, each based on the active substance content in the total composition.

In a further preferred embodiment, the compositions according to the invention comprise at least one derivative of methylated silanol, preferably at least one ester of methylated silanol. Preferred derivatives of methylated silanol are selected from:
sodium mannuronate methylsilanol (Algisium, Exsymol)
methylsilanol mannuronate (Algisium C®, Exsymol)
methylsilanol mannuronate Nylon-12 (Algisium C Powder®, Exsymol)
ascorbylmethylsilanol (Ascorbosilane concentrate C®, Exsymol)
ascorbylmethylsilanol pectinate (Ascorbosilane C®, Exsymol)
dimethyl oxobenzodioxsilane (DSBC®), Exsymol)
dimethyl oxobenzodioxasilane Nylon-12 (DSBC Powder®, Exsymol)
sodium hyaluronate dimethylsilanol (DSH®, Exsymol)
dimethylsilanol hyaluronate (DSHC®, Exsymol)
methysilanol glycyrrhizinate (Glysinol®, Exsymol)
methylsilanolhydroxyproline (Hydroxyprolisilane®, Exsymol)
methylsilanolhydroxyproline aspartate (Hydroxyprolisilane C®, Exsymol)
sodium lactate methylsilanol (Lasilium®, Exsymol)
lactoylmethylsilanol elastinate (Lasilium C®, Exsymol)
dioleyl tocopheryl methylsilanol (Liposiliol C®, Exsymol)
methylsilanol acetylmethionate (Methiosilane®, Exsymol)
acetylmethionylmethylsifanol elastinate (Methiosilane C®, Exsymol)
methylsilanol PEG 7 glyceryl cocoate (Monosiliol®, Exsymol)
methylsilanol tri PEG 7 glyceryl cocoate (Monosiliol C®, Exsymol)
methylsilanol elastinate (Proteosilane C®, Exsymol)
pyrollidone carboxylate caustic methylsilanol (Silhydrate®, Exsymol)
pyrollidone carboxylate copper methylsilanol (Silhydrate C®, Exsymol)
methylsilanolcarboxymethyl theophylline (Theophyllisilane®, Exsymol)
methylsilanecarboxymethyl theophylline alginate (Theophyllisilane C® Exsymol)
methylsilanol acetyltyrosine (Tyrosilane®, Exsymol)
copper acetyl tyrosinate methylsilanol (Tyrosilane C®, Exsymol).
   Sodium hyaluronate dimethylsilanol, dimethylsilanol hyaluronate, methylsilanol mannuronate, methylsilanol hydroxyproline and methylsilanol hydroxyproline aspartate are particularly preferred. In a further preferred embodiment, the inventive compositions comprise at least one derivative of methylated silanol in total amounts of 0.001-5 wt. %, preferably 0.005-1 wt. % and particularly preferably 0.01-0.5 wt. %, in each case based on the active substance in the total composition according to the invention.

In a further preferred embodiment, the inventive compositions comprise phytic acid. Particularly preferred cosmetic or dermatological compositions according to the invention comprise phytic acid in a total quantity of 0.001 to 1 wt. %, preferably 0.01 to 0.5 wt. % and particularly preferably 0.05 to 0.1 wt. %, each based on the total composition.

In a further preferred embodiment, the compositions according to the invention comprise at least one extract of Zea Mays (Corn) Kernel. An inventively particularly preferred extract of Zea Mays Kernel is available under the tradename Deliner from the Coletica company. This extract increases and/or improves the interaction between the extra cellular matrix and the fibroblasts. Particularly preferred cosmetic or dermatological compositions according to the invention comprise at least one active substance selected from Zea Mays (Corn) Kernel in a total quantity of 0.01 to 5 wt. %, preferably 0.1 to 3 wt. %, particularly preferably 1 to 2 wt. %, each based on the content of extract tel quel in the total composition. Particularly preferred cosmetic or dermatological compositions according to the invention comprise at least one active substance selected from Zea Mays (Corn) Kernel in a total quantity of 0.00001 to 1 wt. %, preferably 0.0001 to 0.1 wt. %, particularly preferably 0.001 to 0.05 wt. %, each based on the content of active substance in the total composition.

In a further preferred embodiment, the compositions according to the invention comprise at least one extract from Avena Sativa (Oat) Kernel. An inventively particularly preferred extract of Avena Sativa (Oat) Kernel is available under the tradename Drago Beta Glucan (02/060800) from the Symrise Company. This extract increases and/or improves the interaction between the extra cellular matrix and the fibroblasts. Particularly preferred cosmetic or dermatological compositions according to the invention comprise at least one active substance selected from extracts of Avena Sativa (Oat) Kernel in a total quantity of 0.01 to 5 wt. %, preferably 0.1 to 3 wt. %, particularly preferably 1 to 2 wt. %, each based on the content of extract tel quel in the total composition. Particularly preferred cosmetic or dermatological compositions according to the invention comprise at least one active substance selected from extracts of Avena Sativa (Oat) Kernel in a total quantity of 0.00001 to 1 wt. %, preferably 0.0001 to 0.1 wt. %, particularly preferably 0.001 to 0.05 wt. %, each based on the content of active substance in the total composition.

In a further preferred embodiment, the compositions according to the invention comprise at least one product that is extracted by fermentation from sugared black tea with the two symbiotic microorganisms saccharomyces and xylinum, and which has the INCI name Saccharomyces/Xylinum/Black Tea Ferment. This type of product increases and/or improves the interaction between the extra cellular matrix and the fibroblasts. A particularly preferred product is available under tradename Kombuchka from the Sederma Company (INCI name: Saccharomyces/Xylinum/Black Tea Ferment, Glycerin, Hydroxyethylcellulose). Particularly preferred cosmetic or dermatological compositions according to the invention comprise at least one active substance selected from products that are extracted by fermentation from sugared black tea with the two symbiotic microorganisms saccharomyces and xylinum, and which have the INCI name Saccharomyces/Xylinum/Black Tea Ferment, in a total quantity of 0.01 to 5 wt. %, preferably 0.1 to 3 wt. % and particularly preferably 1 to 2 wt. %, each based on the content of product tel quell in the total composition. Particularly preferred cosmetic or dermatological compositions according to the invention comprise at least one active substance selected from products that are extracted by fermentation from sugared black tea with the two symbiotic microorganisms saccharomyces and xylinum, and which have the INCI name Saccharomyces/Xylinum/Black Tea Ferment, in a total quantity of 0.00001 to 1 wt. %, preferably 0.0001 to 0.1 wt. % and particularly preferably 0.001 to 0.05 wt. %, each based on the content of active substance in the total composition.

In a further preferred embodiment, the compositions according to the invention comprise at least one Pyrus Malus (Apple) Fruit Extract. These types of product increase and/or improve the interaction between the extra cellular matrix and the fibroblasts. Inentively particularly preferred Pyrus Malus (Apple) Fruit Extracts are available under the tradename Ederline from the Seporga Company. The Ederline product comprises phytohormones, isoflavonoids, phytosterols, triterpenoids, tocopherols and natural waxes. Ederline is available firstly in water-soluble form as Ederline-H (INCI: PEG-40 Hydrogenated Castor Oil, PPG-2-Ceteareth-9, Pyrus Malus (Apple) Fruit Extract), secondly in fat-soluble form as Ederline-L (INCI: Hexyldecanol, Pyrus Malus (Apple) Fruit Extract). Particularly preferred cosmetic or dermatological compositions according to the invention comprise the raw material Ederline in quantities of 0.1 to 10 wt. %, preferably 1 to 8 wt. % and particularly preferably 3 to 5 wt. %, each based on the total composition. Particularly preferred cosmetic or dermatological compositions according to the invention comprise an apple core extract in quantities of 0.00001 to 2 wt. %, preferably 0.001 to 1.6 wt. % and particularly preferably 0.03 to 1 wt. %, each based on the content of active substance in the total composition.

In a further preferred embodiment, the compositions according to the invention comprise at least one Nelumbo Nucifera Germ Extract These types of extracts increase and/or improve the interaction between the extra cellular matrix and the fibroblasts. An inventively particularly preferred Nelumbo Nucifera Germ Extract is available under the tradename Lotus Germ Extract with the INCI name Water, Butylene Glycol, Nelumbo Nucifera Germ Extract from the Maruzen Company. Particularly preferred cosmetic or dermatological compositions according to the invention comprise Nelumbo Nucifera Germ Extract in quantities of 0.1 to 10 wt. %, preferably 1 to 8 wt. % and particularly preferably 2 to 3 wt. %, each based on the total composition. Particularly preferred cosmetic or dermatological compositions according to the invention comprise a Nelumbo Nucifera Germ Extract in quantities of 0.00001 to 1 wt. %, preferably 0.0001 to 0.1 wt. % and particularly preferably 0.001 to 0.05 wt. %, each based on the content of active substance in the total composition.

In a further preferred embodiment, the compositions according to the invention comprise at least one extract from red wine. These types of extracts increase and/or improve the interaction between the extra cellular matrix and the fibroblasts. An inventively particularly preferred red wine extract is available under the tradename Sepivinol R from the Seppic Company. Particularly preferred cosmetic or dermatological compositions according to the invention comprise at least one red wine extract in quantities of 0.1 to 10 wt. %, preferably 1 to 8 wt. % and particularly preferably 2 to 3 wt. %, each based on the total composition. Particularly preferred cosmetic or dermatological compositions according to the invention comprise at least one red wine extract in quantities of 0.00001 to 1 wt. %, preferably 0.0001 to 0.1 wt. % and particularly preferably 0.001 to 0.05 wt. %, each based on the content of active substance in the total composition.

In a further preferred embodiment, the compositions according to the invention comprise at least one Vitis Vinifera (Grape) Seed Extract). These types of extracts increase and/or improve the interaction between the extra cellular matrix and the fibroblasts. The Grape Seed Extracts derive particularly preferably from the Chardonnay grape. Inventively particularly preferred Grape Seed Extracts are available under the tradename Herbalia Grape from Cognis or under the tradename Crodarom Chardonnay from Croda. Particularly preferred cosmetic or dermatological compositions according to the invention comprise at least one Grape Seed Extract in quantities of 0.1 to 10 wt. %, preferably 1 to 8 wt. % and particularly preferably 2 to 3 wt. %, each based on the total composition. Particularly preferred cosmetic or dermatological compositions according to the invention comprise a (Grape Seed Extract in quantities of 0.00001 to 1 wt. %, preferably 0.0001 to 0.1 wt. % and particularly preferably 0.001 to 0.05 wt. %, each based on the content of active substance in the total composition.

In a further preferred embodiment, the compositions according to the invention comprise at least one Sambucus Nigra Flower Extract. These types of extracts increase and/or improve the interaction between the extra cellular matrix and the fibroblasts. An inventively particularly preferred Sambucus Nigra Flower Extract is available under the tradename Sambucus AO from the Alpaflor/Centerchem or from Permcos. Particularly preferred cosmetic or dermatological compositions according to the invention comprise at least one Sambucus Nigra Flower Extract in quantities of 0.1 to 10 wt. %, preferably 1 to 5 wt. % and particularly preferably 2 to 3 wt. %, each based on the total composition. Particularly preferred cosmetic or dermatological compositions according to the invention comprise a Sambucus Nigra Flower Extract in quantities of 0.00001 to 1 wt. %, preferably 0.0001 to 0.1 wt. % and particularly preferably 0.001 to 0.05 wt. %, each based on the content of active substance in the total composition.

In a further preferred embodiment, the compositions according to the invention comprise at least one active substance that stimulates the beta-endorphine synthesis in keratinocytes. Inventively particularly preferred stimulants of the beta-endorphine synthesis are selected from mixtures of at least one extract of the leaves of Mentha piperita and at least one extract of coco beans, wherein aqueous, glycolic or aqueous-glycolic preparations of these extract mixtures that are available under the tradenames Caomint, Caophenol, Caobromine, Caospice and Caoorange from Solabia Company are particularly preferred. A further particularly preferred stimulant of the beta-endorphine synthesis is the dipeptide derivative N-acetyl-Tyr-Arg-hexyl-decyl ester with the INCI name Acetyl Dipeptide-1 Cetyl Ester, that is available e.g. as an aqueous preparation under the tradename Calmosensine from Sederma. Further preferred stimulants of the beta-endorphine synthesis are extracts of Helichrysum italicum, e.g. available under the tradename Areaumat Perpetua from Codif, extracts of Crithmum Maritimum, e.g. available under the tradenames Areaumat Samphira and Aroleat Samphira from Codif, extracts of Lavendula stoechas, e.g. available under the tradename Areaumat Lavanda from Codif, extracts of Mentha piperita, as are available e.g. under the tradenames Authenticals of Peppermint (Solabia) and Calmiskin (Silab), glutamylamidoethyl indole, e.g. available under the tradename Glistin from Exsymol, a branched polysaccharide containing rhamnose-, galactose- and glucuronic acid moieties obtained by microbial fermentation with the INCI name Biosaccharide Gum-2, e.g. available under the tradename Rhamnosoft from Solabia, extracts of the seeds of Tephrosia Purpurea with the INCI name Tephrosia Purpurea Seed Extract, e.g. available under the tradename Tephroline from Vincience, mixtures of the oil of Mentha arvensis leaves, lemon peel oil, cypress oil, lavender oil and Cistus Ladaniferus oil with the INCI name Mentha Arvensis Leaf Oil and Citrus Medica Limonum (Lemon) Peel Oil and Cupressus Sempervirens Oil and Lavandula Hybrida Oil and Cistus Ladaniferus Oil, e.g. available under the tradename V-Tonic (Gattefosse), and hexasaccharides according to FR 2842201 as well as any mixture of these active substances.

Particularly preferred cosmetic or dermatological compositions according to the invention comprise at least one active substance for the stimulation of the beta-endorphine synthesis in total quantities of 0.01 to 10 wt. %, preferably 0.1 to 5 wt. % and particularly preferably 1 to 3 wt. %, each based on the commercial product that comprises the active substance, in the total composition according to the invention. Particularly preferred cosmetic or dermatological compositions according to the invention comprise at least one active substance for the stimulation of the beta-endorphine synthesis in total quantities of 0.00001 to 1 wt. %, preferably 0.0001 to 0.1 wt. % and particularly preferably 0.001 to 0.05 wt. %, each based on the content of active substance in the total composition according to the invention.

In a further preferred embodiment, the compositions according to the invention comprise at least one inorganic and/or at least one organic UV filter. The UV filters are liquid or crystalline substances at room temperature which are able to absorb ultra violet radiation and emit the resulting energy in the form of longer wavelength radiation, for example as heat. One differentiates between UVA-filters and UVB-filters. The UV-A and UV-B filters can be used individually as well as in mixtures. According to the invention, it is preferred to use mixtures of filters. The organic UV-filters used according to the invention are selected from derivatives of dibenzoyl methane, cinnamic acid esters, diphenylacrylic acid esters, benzophenone, camphor, p-aminobenzoic acid esters, o-aminobenzoic acid esters, salicylic acid esters, benzimidazoles, symmetrically or unsymmetrically substituted 1,3,5-triazines, monomeric and oligomeric 4,4-diarylbutadienecarboxylic acid esters and -carboxylic acid amides, ketotricyclo(5.2.1.0)decane, benzalmalonic acid esters as well as any mixtures of the cited components. The organic UV-filters can be oil-soluble or water-soluble. According to the invention, particularly preferred oil-soluble UV-filters are 1-(4-tert.-butylphenyl)-3-(4′-methoxyphenyl)propane-1,3-dione (Parsol® 1789), 1-phenyl-3-(4′-isopropylphenyl)-propane-1,3-dione, 3-(4′-methylbenzylidene)-D,L-camphor, 4-(dimethylamino)-benzoic acid 2-ethylhexyl ester, 4-(dimethylamino)benzoic acid 2-octyl ester, 4-(dimethylamino)-benzoic acid amyl ester, 4-methoxycinnamic acid 2-ethylhexyl ester, 4-methoxycinnamic acid propyl ester, 4-methoxycinnamic acid isopentyl ester, 2-cyano-3,3-phenylcinnamic acid 2-ethylhexyl ester (Octocrylene), salicylic acid 2-ethylhexyl ester, salicylic acid 4-isopropylbenzyl ester, salicylic acid homomethyl ester (3,3,5-trimethyl-cyclohexyl salicylate), 2-hydroxy-4-methoxybenzophenone, 2-hydroxy-4-methoxy-4′-methylbenzophenone, 2,2′-dihydroxy-4-methoxybenzophenone, 4-methoxybenzmalonic acid di-2-ethylhexyl ester, 2,4,6-trianilino-(p-carbo-2′-ethyl-1′-hexyloxy)-1,3,5-triazine (Octyl Triazone Uvinul® T 150), dimethicodiethylbenzal malonate (CAS no. 207574-74-1, Parsol® SLX), dioctyl butamido triazone (Uvasorb® HEB), 2,4-bis-[5-1(di-methylpropyl)benzoxazol-2-yl-(4-phenyl)-imino]-6-(2-ethyl hexyl)-imino-1,3,5-triazine (CAS no. 288254-16-0, Uvasorb® K2A) and as well as any mixtures of the cited components. Preferred water-soluble UV filters are 2-phenylbenzimidazole-5-sulfonic acid, phenylene-1,4-bis-(2-benzimidazyl)-3,3′-5,5′-tetrasulfonic acid and their alkali metal, alkaline earth metal, ammonium, alkylammonium, alkanolammonium and glucammonium salts; sulfonic acid derivatives of benzophenone, preferably 2-hydroxy-4-methoxybenzophenone-5-sulfonic acid and its salts; sulfonic acid derivatives of 3-benzylidenecamphor, such as for example 4-(2-oxo-3-bornylidenemethyl)benzene sulfonic acid and 2-methyl-5-(2-oxo-3-bornylidene) sulfonic acid and their salts. Some of the oil-soluble UV-filters can serve as solvents or solubilizers for other UV-filters. Thus, for example, solutions of the UV-A filter 1-(4-tert.-butylphenyl)-3-(4′-methoxyphenyl)propane-1,3-dione (e.g. Parsol® 1789) can be prepared in various UV-B filters. In a further preferred embodiment, the inventive compositions therefore comprise 1-(4-tert.-butylphenyl)-3-(4′-methoxyphenyl)propane-1,3-dione in combination with at least one UV-B filter, selected from 4-methoxycinnamic acid 2-ethylhexyl ester, 2-cyano-3,3-phenylcinnamic acid 2-ethylhexyl ester, salicylic acid 2-ethylhexyl ester and 3,3,5-trimethyl-cyclohexyl salicylate. In these combinations the ratio by weight of the UV-B filter to the 1-(4-tert.-butylphenyl)-3-(4′-methoxyphenyl)propane-1,3-dione is between 1:1 and 10:1, preferably between 2:1 and 8:1, the molar ratio lying correspondingly between 0.3 and 3.8, preferably between 0.7 and 3.0.

The inventively preferred inorganic light stabilizer pigments are finely divided or colloidally dispersed metal oxides and metal salts, e.g. titanium dioxide, zinc oxide, iron oxide, aluminum oxide, cerium oxide, zirconium oxide, silicates (talc) and barium sulfate. Here, the particles should have a mean diameter of less than 100 nm, preferably between 5 and 50 nm and especially between 15 and 30 nm, so-called nanopigments. They can be spherical, however elliptical or other non-spherical shaped particles can also be used. The pigments can also be surface treated, i.e. hydrophilized or hydrophobized. Typical examples are coated titanium dioxides, such as, for example Titandioxid T 805 (Degussa) or Eusolex® T2000 (Merck). Hydrophobic coating agents preferably include silicones and among them specifically trialkoxy octylsilanes or Simethicones. Titanium dioxide and zinc oxide are particularly preferred.

Particularly preferred cosmetic or dermatological compositions according to the invention comprise at least one organic UV-filter in a total quantity of 0.1 to 30 wt. %, preferably 0.5 to 20 wt. %, particularly preferably 1.0 to 15 wt. % and extremely preferably 3.0 to 10 wt. %, each based on the total composition. Particularly preferred cosmetic or dermatological compositions according to the invention comprise at least one inorganic UV-filter in a total quantity of 0.1 to 15 wt. %, preferably 0.5 to 10 wt. %, particularly preferably 1.0 to 5 wt. % and extremely preferably 2.0 to 4.0 wt. %, each based on the total composition.

In a further preferred embodiment, the compositions according to the invention comprise at least one self-tanning active substance. Inventively preferred self-tanning active substances are selected from dihydroxyacetone, tyrosine, tyrosine derivatives, 5,6-dihydroxyindoline and erythrulose. Particularly preferred cosmetic or dermatological compositions according to the invention comprise at least one self-tanning active substance in a total quantity of 0.1 to 15 wt. %, preferably 0.5 to 10 wt. %, particularly preferably 1.0 to 5 wt. % and extremely preferably 2.0 to 4.0 wt. %, each based on the total composition.

In a further preferred embodiment, the compositions according to the invention comprise at least one skin lightening active substance. Inventively preferred skin lightening active substances are selected from ascorbic acid, the esters of ascorbic acid with phosphoric acid and/or organic C₂-C₂₀ carboxylic acids as well as their alkali metal and alkaline earth metal salts, Kojic acid, hydroquinone, arbutin, mulberry tree extract and licorice extract as well as mixtures thereof. The ascorbic acid derivatives as well as Kojic acid are preferred both as a single substance as well as in a mixture. Sodium ascorbyl phosphate, magnesium ascorbyl phosphate, ascorbyl monopalmitate, ascorbyl dipalmitate, ascorbyl monostearate, ascorbyl distearate, ascorbyl monoethylhexanoate, ascorbyl diethylhexanoate, ascorbyl monooctanoate, ascorbyl dioctanoate, ascorbyl monoisostearate and ascorbyl diisostearate are particularly preferred. The inventively extremely preferred ascorbic acid derivatives are sodium ascorbyl phosphate and magnesium ascorbyl phosphate. Particularly preferred stick compositions according to the invention comprise at least one skin lightening active substance in a total quantity of 0.05 to 5 wt. %, preferably 0.1 to 2 wt. %, each based on the total composition.

In a further preferred embodiment, the compositions according to the invention comprise at least one active substance that inhibits the prostaglandin synthesis and/or the leukotriene synthesis. Preferred active substances that inhibit the prostaglandin synthesis are selected from active substances that inhibit the enzyme cyclooxygenase and active substances that inhibit the release of interleukins, in particular interleukin-1-alpha. In the context of the invention, the inhibition of the cyclooxygenase can be understood to mean both a reduction of the amount of this enzyme as well as a lowering of its activity as well as both of these. Preferred active substances that inhibit the leukotriene synthesis are selected from active substances that inhibit the enzyme 5-lipoxygenase. In the context of the invention, the inhibition of the 5-lipoxygenase can be understood to mean both a reduction of the amount of this enzyme as well as a lowering of its activity as well as both of these. Inventively preferred inhibitors of the prostaglandin synthesis, especially inhibitors of the cyclooxygenase and/or the interleukin release, are selected from silymarin that is particularly preferably employed in liposome encapsulated form (available e.g. under the tradename Silymarin Phytosome (INCI: Silybum Marianum Extract and Phospholipids) from Indena SpA. Silymarin represents an active substance concentrate, previously considered a uniform substance, from the fruits of the milk thistle (Silybum marianum). The principal constituents of silymarin are silybin (silymarin I), silychristin (silymarin II), and silydianin, which belong to the group of the flavanolignans. Further inventively preferred inhibitors of the prostaglandin synthesis, especially inhibitors of the cyclooxygenase and/or the interleukin release, are selected from extracts of Centella asiatica, available for example under the name Madecassicoside from DSM, glycyrrethic acid that is particularly preferred in liposome encapsulated form and is available in this form under e.g. the tradename Calmsphere from Soliance, mixtures of corn waxes, extracts of shea butter, and Argania spinosa oil having the INCI name “Spent grain wax and Butyrospermum Parkii (shea butter) extract and Argania Spinosa Kernel Oil,” as available e.g., under the commercial designation Stimu-Tex AS from the Pentapharm company, extracts of Vanilla tahitensis such as those obtainable e.g., under the commercial designation Vanirea (INCI: Vanilla Tahitensis Fruit Extract) from the Solabia company, extracts of olive leaves (INCI: Olea Europaea (Olive) Leaf Extract), as are available particularly under the tradename Oleanoline DPG from Vincience, algin hydrolysates such as those obtainable e.g., under the commercial designation Phycosaccharide, in particular Phycosaccharide Al, from the Codif company, extracts of Bacopa monniera such as those obtainable e.g., under the commercial designation Bacocalmine from the Sederma company, extracts from the robibos plant such as those obtainable e.g., under the commercial name Rooibos Herbasec MPE from the Cosmetochem company, the physiologically compatible salts of sterol sulfates such as those obtainable e.g., under the commercial designation Phytocohesine (INCI: Sodium Beta-Sitosterylsulfate) from the Vincience company, as well as any mixtures of said substances.

Particularly preferred cosmetic or dermatological compositions according to the invention comprise at least one inhibitor of the prostaglandin synthesis in a total quantity of 0.0001 to 10.0 wt. %, preferably 0.001 to 2.0 wt. %, particularly preferably 0.05 to 1 wt. % and extremely preferably 0.1 to 0.5 wt. %, each based on the total composition.

Inventively preferred inhibitors of the leukotriene synthesis, especially inhibitors of the 5-lipoxygenase, are selected from algin hydrolysates, amino dicarboxylic acids with a carbon chain length of 3 to 6 carbon atoms as well as their physiologically compatible salts, N-alkylated C₂-C₁₁ amino acids containing C₁-C₂₂ alkyl groups as well as their physiologically compatible salts, N-acylated C₂-C₁₁ amino acids containing C₂-C₂₂ acyl groups as well as their physiologically compatible salts, yeast extracts, α-bisabolol, α-lipoic acid, allantoin as well as any mixture of these active substances.

In a preferred embodiment, the algin hydrolysates according to the invention are selected from the products that are available e.g. under the tradename Phycosaccharide, especially Phycosaccharide Al, from the Codif Company.

In another preferred embodiment, the inventively preferred amino dicarboxylic acids with a carbon chain length of 3 to 6 carbon atoms are selected from amino malonic acid, amino succinic acid (=aspartic acid), amino glutaric acid and amino adipic acid as well as their physiologically compatible salts. Aspartic acid and their physiologically compatible salts, in particular potassium aspartate and magnesium aspartate, are particularly preferred. The amino dicarboxylic acids with a carbon chain length of 3 to 6 carbon atoms as well as their salts are inventively preferably employed in quantities of 0.01 to 5 wt. %, preferably 0.1 to 2 wt. % and particularly preferably from 0.5 to 1 wt. %, each based on the total composition according to the invention.

In another preferred embodiment, the inventively preferred N-alkylated C₂-C₁₁ amino acids with a C₁-C₂₂ alkyl group are selected from alanine, glutamic acid, pyroglutamic acid, lysine, arginine, histidine, valine, leucine, isoleucine, proline, tryptophan, phenylalanine, methionine, glycine, serine, tyrosine, threonine, cysteine, asparagine and glutamine as well as their physiologically compatible salts, and which possess a C₁-C₂₂ alkyl group on the nitrogen atom of the amino group, selected from a group methyl, ethyl, propyl, butyl, pentyl, hexyl, heptyl, octyl, nonyl, decyl, undecyl, dodecyl (lauryl), tridecyl, tetradecyl (myristyl), pentadecyl, hexadecyl (palmityl, cetyl), heptadecyl, octadecyl (stearyl), nonadecyl, eicosanyl (arachidyl) and behenyl. N-Methylglycine (=sarcosine) is particularly preferred. The N-alkylated C₂-C₁₁ amino acids with a C₁-C₂₂ alkyl group as well as their physiologically compatible salts are inventively preferably employed in quantities of 0.01 to 10 wt. %, preferably 0.1 to 5 wt. % and particularly preferably 0.5 to 2 wt. %, each based on the total composition according to the invention.

In another preferred embodiment, the inventively preferred N-alkylated C₂-C₁₁ amino acids with a C₂-C₂₂ acyl group are selected from glutamic acid, pyroglutamic acid, lysine, arginine, histidine, valine, leucine, isoleucine, proline, tryptophan, phenylalanine, methionine, glycine, serine, tyrosine, threonine, cysteine, asparagines and glutamine as well as their physiologically compatible salts. The amino acids can be used singly or in a mixture. Amino acid mixtures that are obtained from plants, especially corn plants, are particularly suitable according to the invention. The C₂-C₂₂ acyl group, with which the cited amino acids are derivatised on the amino group, is selected from an acetyl, propanoyl, butanoyl, pentanoyl, hexanoyl, heptanoyl, octanoyl, nonanoyl, decanoyl, undecanoyl, lauroyl, tridecanoyl, myristoyl, pentadecanoyl, cetoyl, palmitoyl, stearoyl, elaidoyl, arachidoyl or behenoyl group. Mixtures of C₈-C₁₈ acyl groups are also called cocoyl groups and are likewise preferred substituents. Sodium cocoyl amino acids, sodium octanoyl glutamate, sodium decanoyl glutamate, sodium lauroyl glutamate, sodium myristoyl glutamate, sodium cetoyl glutamate and sodium stearoyl glutamate and the lauroyl derivatives of amino acids obtained from corn plants are particularly preferred. The corn plants, from which the inventively suitable amino acids are obtained, are not subject to any restriction. Oats, wheat, barley and rye, for example are suitable; oats are particularly suitable. A particularly preferred 5-lipoxygenase inhibitor is the commercial product Seppicalm from Seppic with the INCI name “Sodium Cocoyl Aminoacids, Sarcosine, Potassium Aspartate, Magnesium Aspartate”. The N-alkylated C₂-C₁₁ amino acids with a C₂-C₂₂ acyl group as well as their physiologically compatible salts are inventively preferably employed in quantities of 0.01 to 10 wt. %, preferably 0.1 to 5 wt. % and particularly preferably 0.5 to 2 wt. %, each based on the total topical composition.

In a further preferred embodiment, the inventively preferred yeast extracts are employed as the 5-lipoxygenase inhibitors in amounts of 0.001 to 5 wt. %, preferably 0.01 to 2 wt. % and particularly preferably 0.1 to 1 wt. %, in each case based on the extract tel quel in the total composition according to the invention. A particularly preferably employed commercial product is Drieline (INCI name “Sorbitol, Yeast Extract”), available from Lanatech.

In a further preferred embodiment, the inventively preferred 5-lipoxygenase inhibitor α-Bisabolol is employed in amounts of 0.001 to 5 wt. %, preferably 0.01 to 2 wt. % and particularly preferably 0.1 to 1 wt. %, in each case based on the total topical composition.

In a further preferred embodiment, the inventively preferred 5-lipoxygenase inhibitor α-lipoic acid is employed in amounts of 0.001 to 5 wt. %, preferably 0.01 to 2 wt. % and particularly preferably 0.1 to 1 wt. %, in each case based on the total topical composition.

In a further preferred embodiment, the inventively preferred 5-lipoxygenase inhibitor allantoin is employed in amounts of 0.001 to 5 wt. %, preferably 0.01 to 2 wt. % and particularly preferably 0.1 to 1 wt. %, in each case based on the total topical composition.

In a further preferred embodiment, the physiologically compatible salts of the sterol sulfates that are inventively preferred as the 5-lipoxygenase inhibitors are selected from the salts of β-sitosterol sulfate, ergosterol sulfate, stigmasterol sulfate, cholesterol sulfate and lanosterol sulfate. The salts of β-sitosterol sulfate are particularly preferred. The sterol sulfate salts are employed in amounts of 0.001 to 5 wt. %, preferably 0.01 to 2 wt. % and particularly preferably 0.1 to 1 wt. %, in each case based on the total topical composition. Here, the sterol sulfate salts can be employed both singly as well as in any mixture. A particularly preferably employed commercial product is Phytocohesine (INCI name “Sodium Beta-Sitosteryl Sulfate”), available from the Vincience Company.

The physiologically compatible salts of the abovementioned 5-lipoxygenase inhibitors are selected from the ammonium, alkali metal, magnesium, calcium, aluminum, zinc and manganese salts. The sodium, potassium, magnesium, aluminum, zinc and manganese salts are preferred.

Particularly preferred cosmetic or dermatological compositions according to the invention comprise at least one inhibitor of the leukotriene synthesis in a total quantity of 0.0001 to 10.0 wt. %, preferably 0.001 to 2.0 wt. %, particularly preferably 0.05 to 1 wt. % and extremely preferably 0.1 to 0.5 wt. %, each based on the total composition.

In a further preferred embodiment, the compositions according to the invention comprise at least one sebum-regulating active substance. Sebum-regulating active substances preferred according to the invention are selected from 10-hydroxydecanoic acid, sebacic acid, azelaic acid, and esters of azelaic acid, in particular potassium azeloyl diglycinate, 1,10-decanediol and at least one extract of Spiraea Ulmaria as well as mixtures of the abovementioned substances. Preferred mixtures are available for example, as the commercial product Acnacidol PG (Propylene Glycol, 10-Hydroxydecanoic acid, Sebacic acid, 1,10-Decandiol) from Vincience. A preferred extract of Spiraea Ulmaria is comprised e.g. in the product Seboregul 2 from the Silab Company. Potassium azeloyl diglycinate is comprised e.g. in the product Azeloglicina from the Sinerga Company. Particularly preferred cosmetic or dermatological compositions according to the invention comprise at least one sebum-regulating active substance in total quantities of 0.00001 to 10 wt. %, preferably 0.01 to 5 wt. % and particularly preferably 0.1 to 1-2 wt. %, each based on the active substance in the total composition according to the invention.

Particularly preferred inventive stick compositions comprise at least one moisture-donating active substance. Moisture-donating active substances preferred according to the invention are selected from deoxy sugars, particularly preferably rhamnose and fucose, polysaccharides that contain at least one deoxy sugar moiety, particularly preferably from the commercial products Fucogel® (INCI name: Biosaccharide Gum-1) from Solabia, Rhamnosoft® (INCI name: Biosaccharide Gum-2) from Solabia, Fucogenol® (INCI name: Biosaccharide Gum-3) from Solabia, and Glycofilm® (INCI name: Biosaccharide Gum-4) from Solabia, also mixtures of the aforesaid polysaccharides containing at least one deoxy sugar moiety, for example the mixture of Biosaccharide Gum-2 and Biosaccharide Gum-3 obtainable as a commercial product Elastinol Plus® from Solabia, furthermore urea, N,N′-bis(2-hydroxyethyl)urea, also alkyl or hydroxyalkyl-substituted urea of the general Formula (UREA),

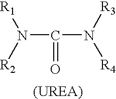

in which R₁, R₂, R₃ and R₄ independently of each other stand for a hydrogen atom, a methyl, ethyl, n-propyl, isopropyl, n-butyl, sec.-butyl, tert.-butyl or C₂-C₆ hydroxyalkyl group that is substituted with 1 to 5 hydroxyl groups or C₁-C₄ hydroxyalkyl groups, with the proviso that at least one of the R¹-R⁴ groups represents a C₂-C₆ hydroxyalkyl group that is substituted with 1 to 5 hydroxyl groups or C₁-C₄ hydroxyalkyl groups, in particular (2-hydroxyethyl)urea and N,N′-bis(2-hydroxyethyl)urea, betaine (Me₃N⁺—CH₂—COO″), chitosans, glycosamino glycans, particularly preferably hyaluronic acid, dextran, dextran sulfate, chondroitin-4-sulfate and chondroitin-6-sulfate as well as any mixture of these substances.

Particularly preferred inventive stick compositions according to the invention comprise at least one moisture-donating active substance in a total quantity of 0.001 to 10 wt. %, preferably 0.01 to 5 wt. % and particularly preferably 0.1 to 1 or 2 wt. %, each based on the total stick composition. Further particularly preferred inventive stick compositions comprise at least one prebiotic active substance. According to the invention, prebiotic active substances are understood to mean those components that only inhibit or at least predominantly inhibit unwanted germs of the skin microflora, but not the wanted, i.e. the germs that belong to a healthy skin microflora. The active substances disclosed in the Offenlegungsschriften DE 10333245 and DE 10 2004 011 968 as prebiotically active are explicitly incorporated herein; they include conifer extracts, especially from the group of the Pinaceae, and plant extracts from the group of the Sapindaceae, Araliaceae, Lamiaceae and Saxifragaceae, especially extracts from Picea spp., Paullinia sp., Panax sp., Lamium album or Ribes nigrum as well as mixtures of these substances. Particularly preferred stick compositions according to the invention comprise at least one prebiotic active substance in a total quantity of 0.01 to 10 wt. %, preferably 0.1 to 5 wt. % and particularly preferably 0.5 to 2 wt. %.

Further particularly preferred inventive stick compositions comprise at least one colored, coloring, matt or glossy pigment. Preferred pigments of this type can be inorganic or organic. Further preferred pigments possess an average particle size of 0.1-200 μm, preferably 0.5-100 μm, particularly preferably 1-50 μm and extremely preferably 2-30 μm. Particularly preferred inorganic pigments are selected from the oxides of silicon, titanium, iron, zinc, zirconium, magnesium, cerium and bismuth, from bismuth oxychloride, boron nitride, mica, fluorite and water-insoluble pearlescent pigments which can be coated with at least one inorganic and/or organic compound. The dyes and color pigments can be selected from the corresponding positive list of the cosmetic ordinance or from the EU list of cosmetic dyes. In the majority of cases they are identical to the dyes approved for foodstuffs. Particularly preferred color pigments are for example titanium dioxide, mica, iron oxides (e.g. Fe₂O₃, Fe₃O₄, FeO(OH)) and/or tin oxide. Particularly preferred dyes are for example Carmin, Berlin Blue, Chromoxide green, Ultramarine blue and/or Manganese violet. It is particularly advantageous to choose the dyes and/or color pigments from the following list. The Colour Index Numbers (CIN) are taken from the Rowe Colour Index, 3rd edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic | 10316 | yellow |
| acid | | |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Cerium Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfonic acid diethylamide- | 12490 | red |
| 1′-phenylazo)-3-hydroxy-5″-chloro-2″,4″- | | |
| dimethoxy-2-naphthoic acid anilide | | |
| Disperse Yellow 16 | 12700 | yellow |
| 1-(4-Sulfo-1-phenylazo)-4-amino-benzene- | 13015 | yellow |
| 5-sulfonic acid | | |
| 2,4-Dihydroxy-azobenzene-4′-sulfonic acid | 14270 | orange |
| 2-(2,4-Dimethylphenylazo-5-sulfonic acid)-1- | 14700 | red |
| hydroxynaphthalene-4-sulfonic acid | | |
| 2-(4-Sulfo-1-naphthylazo)-1-naphthene-4- | 14720 | red |
| sulfonic acid | | |
| 2-(6-Sulfo-2,4-xylylazo)-1-naphthene-5- | 14815 | red |
| sulfonic acid | | |
| 1-(4′-Sulfophenylazo)-2-hydroxynaphthalene | 15510 | orange |
| 1-(2-Sulfonic acid-4-chloro-5-carboxylic | 15525 | red |
| acid-1-phenylazo)-2-hydroxynaphthalene | | |
| 1-(3-Methyl-phenylazo-4-sulfonic acid)-2- | 15580 | red |
| hydroxynaphthalene | | |
| 1-(4′,(8′)-Sulfonic acid naphthylazo)-2- | 15620 | red |
| hydroxynaphthalene | | |
| 2-Hydroxy-1,2′-azonaphthalene-1′-sulfonic | 15630 | red |
| acid | | |
| 3-Hydroxy-4-phenylazo-2-naphthylcarboxylic | 15800 | red |
| acid | | |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)- | 15865 | red |
| 2-hydroxyo-naphthalene-3-carboxylic acid | | |
| 1-(2-Sulfo-1-naphthylazo)-2- | 15880 | red |
| hydroxynaphthalene-3-carboxylic acid | | |
| 1-(3-Sulfo-1-phenylazo)-2-naphthene-6- | 15980 | orange |
| sulfonic acid | | |
| 1-(4-Sulfo-1-phenylazo)-2-naphthene-6- | 15985 | yellow |
| sulfonic acid | | |
| Altura Red | 16035 | red |
| 1-(4-Sulfo-1-naphthylazo)-2-naphthene-3,6- | 16185 | red |
| disulfonic acid | | |
| Acid Orange 10 | 16230 | orange |
| 1-(4-Sulfo-1-naphthylazo)-2-naphthene-6,8- | 16255 | red |
| disulfonic acid | | |
| 1-(4-Sulfo-1-naphthylazo)-2-naphthene- | 16290 | red |
| 3,6,8-trisulfonic acid | | |
| 8-Amino-2-phenylazo-1-naphthene-3,6- | 17200 | red |
| disulfonic acid | | |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5- | 19140 | yellow |
| hydroxy-pyrazolone-3-carboxylic acid | | |
| Pigment Yellow 16 | 20040 | yellow |
| 2,6-(4′-Sulfo-2″,4″-dimethyl)-bis- | 20170 | orange |
| phenylazo)1,3-dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′- | 27755 | black |
| naphthylazo]-1-hydroxy-7-amino-naphthalene- | | |
| 3,6-disulfonic acid | | |
| 4′-[(4″-Sulfo-1″-phenylazo)-7′-sulfo-1′-naphthylazo]- | 28440 | black |
| 1-hydroxy-8-acetyl-amino-naphthalene-3,5- | | |
| disulfonic acid | | |
| Direct Orange 34, 39, 44, 46, 60 | 40215 | orange |
| Food Yellow | 40800 | orange |
| trans-β-Apo-8′-carotenealdehyd (C₃₀) | 40820 | orange |
| trans-Apo-8′-carotenic acid (C₃₀) ethyl ester | 40825 | orange |
| Canthaxanthine | 40850 | orange |
| Acid Blue 1 | 42045 | blue |
| 2,4-Disulfo-5-hydroxy-4′-4″-bis- | 42051 | blue |
| diethylamino)triphenylcarbinol | | |
| 4-[(-4-N-Ethyl-p-sulfobenzylamino)-phenyl-(4- | 42053 | green |
| hydroxy-2-sulfophenyl)-(methylene)-1-(N-ethyl-N- | | |
| p-sulfobenzyl)-2,5-cyclohexadienimine] | | |
| Acid Blue 7 | 42080 | blue |
| N-Ethyl-p-sulfobenzyl-amino)-phenyl-(2- | 42090 | blue |
| sulfophenyl)-methylene-(N-ethyl-N-p-sulfo-benzyl)- | | |
| 2,5-cyclohexadienimine | | |
| Acid Green 9 | 42100 | green |
| Diethyl-disulfobenzyl-di4-amino-2-chloro-di2- | 42170 | green |
| methylfuchsonimmonium | | |
| Basic Violet 14 | 42510 | violet |
| Basic Violet 2 | 42520 | violet |
| 2′-Methyl-4′-(N-ethyl-N-m-sulfobenzyl)-amino-4″-(N- | 42735 | blue |
| diethyl)-amino-2-methyl-N-ethyl-N-m-sulfobenzyl- | | |
| fuchsonimmonium | | |
| 4′-(N-Dimethyl)amino-4″-(N-phenyl)-aminonaphtho-N- | 44045 | blue |
| dimethyl-fuchsonimmonium | | |
| 2-Hydroxy-3,6-disulfo-4,4′-bis- | 44090 | green |
| dimethylaminonaphthofuchsonimmonium | | |
| Acid Red 52 | 45100 | red |
| 3-(2′-Methylphenylamino)-6-(2′-methyl-4′- | 45190 | violet |
| sulfophenylamino)-9-(2″-carboxyphenyl)- | | |
| xanthenium salt | | |
| Acid Red 50 | 45220 | red |
| Phenyl-2-oxyfluorone-2-carboxylic acid | 45350 | yellow |
| 4,5-Dibromfluorescein | 45370 | orange |
| 2,4,5,7-Tetrabromfluorescein | 45380 | red |
| Solvent Dye | 45396 | orange |
| Acid Red 98 | 45405 | red |
| 3′,4′,5′,6′-Tetrachloro-2,4,5,7-tetrabromfluorescein | 45410 | red |
| 4,5-Diiodfluorescein | 45425 | red |
| 2,4,5,7-Tetraiodfluorescein | 45430 | red |
| Quinophthalone | 47000 | yellow |
| Quinophthalone disulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum | 58000 | red |
| complex | | |
| 3-Oxypyrene-5,8,10-sulfonic acid | 59040 | green |
| 1-Hydroxy-4-N-phenyl-aminoanthraquinone | 60724 | violet |
| 1-Hydroxy-4-(4′-methylphenylamino)- | 60725 | violet |
| anthraquinone | | |
| Acid Violet 23 | 60730 | violet |
| 1,4-Di(4′-methylphenylamino)-anthraquinone | 61565 | green |
| 1,4-Bis-(o-sulfo-p-toluidino)-anthraquinone | 61570 | blue |
| Acid Blue 80 | 61585 | blue |
| Acid Blue 62 | 62045 | blue |
| N,N′-Dihydro-1,2,1′,2′-anthraquinone azine | 62045 | blue |
| Vat Blue 6; Pigment Blue 64 | 69825 | blue |
| Vat Orange 7 | 71105 | orange |
| Indigo | 73000 | blue |
| Indigo disulfonic acid | 73015 | blue |
| 4,4′-Dimethyl-6,6-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated Phthalocyanine | 74260 | green |
| Natural Yellow 6, 19; Natural Red 1 | 75100 | yellow |
| Bixin, Nor-Bixin | 75120 | orange |
| Lycopine | 75125 | yellow |
| trans-alpha-, beta- or gamma-Carotene | 75130 | orange |
| Keto- and/or Hydroxyl derivatives of Carotene | 75135 | yellow |
| Guanine or Pearlescent agent | 75170 | white |
| 1,7-Bis-(4-hydroxy-3-methoxyphenyl)1,6- | 75300 | yellow |
| heptadiene-3,5-dione | | |
| Complex salt (Na, Al, Ca) of Carminic acid | 75470 | red |
| Chlorophyll a and b; Copper compounds of | 75810 | green |
| Chlorophyll and Chlorophylline | | |
| Aluminum | 77000 | white |
| Clay hydrate | 77002 | white |
| Water-containing Aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Hydrocarbon | 77266 | black |
| Pigment Black 9 | 77267 | black |
| Carbo medicinalis vegetabilis | 77268:1 | black |
| Chromium oxide | 77288 | green |
| Chromium oxide, hydrated | 77289 | green |
| Pigment Blue 28, Pigment Green 14 | 77346 | green |
| Pigment Metal 2 | 77400 | brown |
| Gold | 77480 | brown |
| Iron oxides and iron hydroxides | 77489 | orange |
| Iron oxide | 77491 | red |
| Iron oxide hydrate | 77492 | yellow |
| Iron oxide | 77499 | black |
| Mixtures of Iron(II) and Iron(III) | 77510 | blue |
| hexacyanoferrate | | |
| Pigment White 18 | 77713 | white |
| Manganese ammonium diphosphate | 77742 | violet |
| Manganese phosphate; Mn₃(PO₄)₂ 7H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)-isoalloxazine, | | yellow |
| Lactoflavine | | |
| Caramel | | brown |
| Capsanthin, Capsorubin | | orange |
| Betanin | | red |
| Benzopyrylium salts, Anthocyans | | red |
| Aluminum-, Zink-, Magnesium- and Calcium | | white |
| stearate | | |
| Bromothymol blue | | blue |
| Bromocresol green | | green |
| Acid Red 195 | | red |

It can be further preferred according to the invention to choose one or more substances as the dye from the following group: 2,4-Dihydroxyazobenzene, 1-(2′-chloro-4′-nitro-1′-phenylazo)-2-hydroxynaphthalene, cerium red, 2-(4-sulfo-1-naphthylazo)-1-naphthene-4-sulfonic acid, calcium salt of hydroxy-1,2′-azo-naphthalene-1′-sulfonic acid, calcium- and barium salts of 1-(2-sulfo-4-methyl-1-phenylazo)-2-naphthyl carboxylic acid, calcium salt of 1-(2-sulfo-1-naphthylazo)-2-hydroxynaphthalene-3-carboxylic acid, aluminum salt of 1-(4-sulfo-1-phenylazo)-2-naphthene-6-sulfonic acid, aluminum salt of 1-(4-sulfo-1-naphthylazo)-2-naphthene-3,6-disulfonic acid, 1-(4-sulfo-1-naphthylazo)-2-naphthene-6,8-disulfonic acid, aluminum salt of 4-(4-sulfo-1-phenylazo)-1-(4-sulfophenyl)-5-hydroxy-pyrazolone-3-carboxylic acid, aluminum- and zirkonium salts of 4,5-dibromofluorescein, aluminum- and zirkonium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalone disulfonic acid, aluminum salt of indigo disulfonic acid, red and black iron oxide (CIN: 77 491 (red) and 77 499 (black)), iron oxide hydrate (CIN: 77 492), manganese ammonium diphosphate and titanium dioxide. Additional inventively preferred dye pigments are oil-soluble natural dyes, such as, for example, paprika extracts, β-carotene or cochineal. Inventively preferred pearlescent materials are selected from natural pearlescent pigments such as e.g. “fish silver” (guanine/hypoxanthine mixed crystals from fish scales) and mother of pearl (ground mussel shells), monocrystalline pearlescent pigments such as e.g. bismuth oxychloride (BiOCl), and layered substrate pigments, e.g. mica/metal oxides. The basis for pearlescent pigments are for example powdered pigments or castor oil dispersions of bismuth oxychloride and/or titanium dioxide as well as bismuth oxychloride and/or titanium dioxide on mica. The lustrous pigment listed under CIN 77163 for example is particularly preferred. Furthermore, the following pearlescent pigment types based on metal oxide-coated mica are inventively preferred:

| Group | Coating/Coating Thickness | Color |
|---|---|---|
| Silverwhite | TiO₂: 40-60 nm | Silver |
| Pearlescent | | |
| Pigments | | |
| Interference Pigments | TiO₂: 60-80 nm | yellow |
| | TiO₂: 80-100 nm | red |
| | TiO₂: 100-140 nm | blue |
| | TiO₂: 120-160 nm | green |
| Colour Luster Pigments | Fe₂O₃ | bronze |
| | Fe₂O₃ | copper |
| | Fe₂O₃ | red |
| | Fe₂O₃ | red violet |
| | Fe₂O₃ | red green |
| | Fe₂O₃ | black |
| Two Tone Pigments | TiO₂/Fe₂O₃ | gold tones |
| | TiO₂/Cr₂O₃ | green |
| | TiO₂/Berlin Blue | deep blue |
| | TiO₂/Carmine | red |

According to the invention, particular preference is given, for example, to the pearlescent pigments obtainable from Merck under the trade names Timiron, Colorona or Dichrona. Naturally, the list of the cited pearlescent pigments is not intended to be limitative. In the context of the present invention, advantageous pearlescent pigments are obtainable from numerous known methods. For example, apart from mica, other substrates can also be coated with additional metal oxides, such as e.g. silica and the like. SiO₂ particles advantageously coated with e.g. TiO₂ and Fe₂O₃ (“Ronasphere”), commercialised by Merck, are particularly suitable for the optical reduction of fine wrinkles. Moreover, it can be inventively preferred to completely forego the use of a substrate such as mica. Pearlescent pigments that are manufactured using SiO₂ are particularly preferred. These pigments, which can additionally have goniochromatic effects, are available for example under the trade name Sicopearl Fantastico from BASF. Also preferred are pigments based on titanium dioxide-coated calcium sodium borosilicate from Engelhard/Mearl. They can be obtained under the name Reflecks. Due to their particle size of 40-180 μm, they exhibit a glitter effect in addition to the color. Furthermore, effect pigments, available under the trade name Metasomes Standard/Glitter in various colors (yellow, red, green, blue) from Flora Tech, are also particularly advantageous. Here, the glitter particles are in mixtures with various auxiliaries and dyes (such as for example with the dyes with the Colour Index (CI) numbers 19140, 77007, 77289, 77491). The dyes and pigments can exist both singly as well as in a mixture and be coated onto one another, wherein various color effects are generally generated by different coating thicknesses. Further preferred pigments are selected from colored and colorless pigments. Some of the pigments cited below also serve as UV absorbers. Particularly preferred colored pigments are selected from the iron oxides with the Color Index numbers C177491 (iron oxide red), C177492 (iron oxide hydrate yellow) and C177499 (iron oxide black), from C177891 (titanium dioxide) and carbon black. A particularly preferred pigment is the commercial product SUNPMMA-S and SUNSIL Tin 30 from Sunjin Chemicals Co. with a mean particle size of 5-10 μm or 2-7 μm. Particularly preferred inorganic pigments are coated. The coating can be made with inorganic and/or organic compounds. Inorganic pigments having an inorganic coating are inventively particularly preferred. Extremely preferred pigments of this type are selected from silicon dioxide particles that are coated with titanium dioxide and/or iron oxides. A particularly preferred pigment of this type is the commercial product Ronasphere® LDP from Merck KGaA. This product concerns spherical silicon dioxide particles that are coated with titanium dioxide and iron oxide. Ronasphere® LDP has a mean particle size of 4-7 μm. Inorganically coated mica pigments that have no pearlescense are also preferred. Further preferred inorganically coated inorganic pigments are mica pigments that are coated with titanium dioxide in various coating thicknesses, for example the products of the Timiron®-Series from Rona/Merck KGaA, in particular pigments from the product lines Timiron® MP, Timiron® Super, Timiron® Starlight and Timiron® Silk. The mean particle sizes of the cited products are 5-60 μm or 10-60 μm or 10-125 μm or 5-25 μm. Mica particles with a coating of titanium dioxide and iron oxide are likewise preferred, e.g. the commercial products Timiron® MP-20, MP-24, MP-25, MP-28, MP-29, MP-60 and MP-65. Mica particles coated with titanium dioxide and/or red and/or black iron oxide are also preferred according to the invention, e.g. the products of the Colbrona® range. Further preferred pigments are mica pigments coated with silica, e.g. the commercial product Micronasphere® M. Further inventively preferred pigments are inorganically coated inorganic pigments, whose coating contains a 0.1-1 wt. % tin oxide content. Inventively preferred pigments are also inorganic pigments coated with organic substances. Preferred examples of them are titanium dioxide pigments coated with aluminum stearate (e.g. the commercial product MT 100 T from Tayca), zinc oxide coated with dimethylpolysiloxane (Dimethicone), boron nitride coated with Dimethicone (Tres BN® UHP 1106 from Carborundum), titanium dioxide coated with a mixture of dimethylpolysiloxane and silica gel (Simethicone) and aluminum oxide hydrate (alumina) (Eusolex® T 2000 from Merck), titanium dioxide coated with octylsilanol or spherical polyalkylsesquisiloxane particles (Aerosil® R972 from Degussa). Another particularly preferred pigment is the commercial product SB-705 from the Miyoshi Kasei Company, a spherical silica gel with the INCI name Silica that has a mean particle size of 5-6 μm and a surface of about 600 m²/g. Particularly preferred inventive stick compositions according to the invention comprise at least one colored, coloring, matting or lustrous pigment in a total quantity of 0.1 to 30 wt. %, preferably 0.5 to 15 wt. %, particularly preferably 1.0 to 10 wt. % and extremely preferably 2 to 5 wt. %, each based on the total weight of the inventive composition.

A further subject matter of the present invention is a cosmetic, non-therapeutic process for diminishing body odor, wherein a cosmetic composition according to one of the patent claims 1-33 is applied onto the skin, in particular onto the skin of the armpits.

A further subject matter of the present invention is a cosmetic, non-therapeutic process for putting on makeup and/or matting and/or for putting on matting makeup on the skin and/or the mucosa, wherein a cosmetic composition according to one of the patent claims 1-33 is applied onto the skin or mucosa.

A further subject matter of the present invention is a cosmetic, non-therapeutic process for concealing tiny wrinkles, wrinkles or fine lines, wherein a cosmetic composition according to one of the patent claims 1-33 is applied onto the skin or mucosa.

A further subject matter of the present invention is a process for manufacturing a stick according to one of the claims 1-33, wherein the wax and oil components are heated together with the oil-in-water emulsifier(s) and the water-in-oil emulsifier(s) to 90-95° C. and melted, the water, likewise heated to 90-95° C. and containing the water-soluble active substances and ingredients, is then added with vigorous stirring, optional additional ingredients are mixed in, the mixture is then cooled to a suitable filing temperature, filled into suitable dispensing forms and allowed to solidify at room temperature by static cooling (without further stirring).

While particular embodiments of the present invention have been illustrated and described, it would be obvious to those skilled in the art that various other changes and modifications can be made without departing from the spirit and scope of the invention.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

The following Examples further illustrate the preferred embodiments within the scope of the present invention, but are not intended to be limiting thereof. It is understood that the examples and embodiments described herein are for illustrative purposes only and that various modifications or changes in light thereof will be suggested to one skilled in the art without departing from the scope of the present invention. The appended claims therefore are intended to cover all such changes and modifications that are within the scope of this invention.

### EXAMPLES

The following examples are intended to illustrate the subject matter of the present invention in more detail, without limiting it in any way.

TABLE 6
Antiperspirant stick according to the invention (quantities in wt. %)

| | No. 1 | Nr. 2 | Nr. 3 | Nr. 4 |
|---|---|---|---|---|
| Cutina AGS | 2.40 | 2.40 | 2.40 | 2.40 |
| Cutina FS45 | 2.75 | 2.75 | 2.75 | 2.75 |
| Eumulgin B1 | 1.30 | 0.90 | 0.50 | 0.25 |
| Eumulgin B2 | 0 | 0.35 | 0.50 | 0 |
| Eumulgin B3 | 0 | 0 | 0 | 0.50 |
| Finsol^{y} TN | 6.00 | 6.00 | 0 | 0 |
| Crodamol ML | 0 | 0 | 6.00 | 6.00 |
| Novata AB | 4.00 | 4.00 | 4.00 | 4.00 |
| Cutina CP | 5.00 | 5.00 | 5.00 | 5.00 |
| Cutina HR | 4.00 | 4.00 | 4.00 | 4.00 |
| Kesterwachs K62 | 5.00 | 5.00 | 5.00 | 5.00 |
| Talc | 10.00 | 10.00 | 10.00 | 10.00 |
| Locron L [ACH-50% | 40.00 | 0 | 40.00 | 0 |
| Reza! 36 GP SUF | 0 | 22.00 | 0 | 22.00 |
| Tinogard Q | 0.05 | 0.05 | 0.05 | 0.05 |
| 1,2-Propanediol | 10.00 | 10.00 | 10.00 | 10.00 |
| Water, deionized | 8.40 | 26.45 | 8.70 | 26.95 |
| Perfume | 1.10 | 1.10 | 1.10 | 1.10 |
| Total | 100.00 | 100.00 | 100.00 | 100.00 |

List of Raw Materials

| | | |
|---|---|---|
| Crodamol ML | MYRISTYL LACTATE | Croda |
| Cutina AGS | GLYCOL DISTEARATE | Cognis |
| Cutina FS45 | PALMITIC ACID, STEARIC ACID | Cognis |
| Eumulgin B1 | CETEARETH-12 | Cognis |
| Eumulgin B2 | CETEARETH-20 | Cognis |
| Eumulgin B3 | CETEARETH-30 | Cognis |
| Finsolv TN | C12-15 ALKYL BENZOATE | Finetex |
| Novata AB | COCOGLYCERIDES | Cognis |
| Cutina CP | CETYL PALMITATE | Cognis |
| Cutina HR | HYDROGENATED CASTOR OIL | Cognis |
| Kesterwachs K62 | CETEARYL BEHENATE | Koster |
| | | Keunen |
| Locron L [ACH-50% | ALUMINUM CHLOROHYDRATE | Clariant |
| Reza! 36 GP SUF | ALUMINUM ZIRCONIUM | Reheis |
| Tinogard Q | TRIS (TETRAMETHYL- | Ciba Spec. |
| | HYDROXYPYRIDINO | |

What is claimed:

1. A cosmetic or dermatological stick in the form of an oil-in-water dispersion/emulsion, comprising
a) at least one wax component with a melting point >50° C., which is not to be apportioned to the components b) or c),
b) at least one non-ionic oil-in-water emulsifier with an HLB value of more than 7 within an oil-in-water emulsifier system with an average HLB value in the range of 11-17,
c) at least one non-ionic water-in-oil emulsifier with an HLB value of more than 1.0 and less than/equal to 7.0, which solely with water or with water in the presence of a hydrophilic emulsifier can form liquid crystalline structures, as consistency providers and/or water binders,
d) at least one oil that is a liquid under normal conditions and is neither a fragrance component nor essential oil, wherein the (average) solubility parameter of the totality of the comprised oils differs by no more than −1.0 (cal/cm³)^{0.5} resp.+1.0 (cal/cm³)^{0.5} from the (average) solubility parameter of the water-in-oil emulsifier/of the water-in-oil emulsifiers,
e) at least one water-soluble polyhydric C₂-C₉ alkanol containing 2-6 hydroxyl groups and/or at least one water-soluble polyethylene glycol having 3-20 ethylene oxide units,
f) 5-70 wt. % water, relative to the total composition,
g) at least one cosmetic or dermatological active substance.

2. The stick according to claim 1, wherein the wax component a) is selected from esters of a saturated, monohydric C₁₂-C₆₀ alkanol and a saturated C₈-C₃₆ monocarboxylic acid, cetyl behenate, stearyl behenate, C₂₀-C₄₀ alkyl stearate, glycerine triesters of saturated linear C₁₂-C₃₀ carboxylic acids that can be hydroxylated, candelilla wax, carnauba wax, beeswax, saturated linear C₁₄-C₃₆ carboxylic acids as well as mixtures thereof.

3. The stick according to claim 1, wherein the ester(s) of a saturated, monohydric C₁₂-C₆₀ alkanol and a saturated C₈-C₃₆ monocarboxylic acid is/are comprised in total amounts of 2-10 wt. % based on the total composition.

4. The stick according to claim 1, wherein the non-ionic oil-in-water emulsifier b) having an HLB value of greater than 7 is selected from
ethoxylated C₈-C₂₄ alkanols with an average of 5-100 mol ethylene oxide per mol,
ethoxylated C₈-C₂₄ carboxylic acids with an average of 5-100 mol ethylene oxide per mol,
silicone copolyols with ethylene oxide units or with ethylene oxide- and propylene oxide units,
alkyl mono- and -oligoglycosides with 8 to 22 carbon atoms in the alkyl residue and their ethoxylated analogs,
ethoxylated sterols,
partial esters of polyglycerines with 2 to 10 glycerine units and esterified with 1 to 4 saturated or unsaturated, linear or branched C₈-C₂₂ fatty acid residues, as long as they have an HLB value of more than 7,
as well as mixtures of the above cited substances.

5. The stick according to claim 1, wherein the water-in-oil emulsifier c) is selected from:
the mono- and diesters of ethylene glycol with linear saturated and unsaturated fatty acids having 12-30, particularly 14-22 carbon atoms, which can be hydroxylated,
the mono-, di-, tri- and tetraesters of pentaerythritol with linear saturated and unsaturated fatty acids having 12-30 or 14-22 carbon atoms, which can be hydroxylated, as well as mixtures thereof,
linear, saturated C₁₂-C₃₀ alkanols,
glycerine mono- and di-esters of linear, saturated and unsaturated C₁₂-C₃₀ carboxylic acids, which can be hydroxylated,
polyglycerine esters of linear, saturated and unsaturated C₁₂-C₃₀ carboxylic acids, which can be hydroxylated, with 2-10 glycerine units and a partial or complete degree of esterification,
propylene glycol mono- and -diesters of linear, saturated and unsaturated C₁₂-C₃₀ carboxylic acids, which can be hydroxylated,
sorbitan mono-, di-, and -triesters of linear, saturated and unsaturated C₁₂-C₃₀ carboxylic acids, which can be hydroxylated,
methyl glucose mono- and -diesters of linear, saturated and unsaturated C₁₂-C₃₀ carboxylic acids, which can be hydroxylated,
sterols,
alkanols and carboxylic acids each having 8-24 carbon atoms, in particular 16-22 carbon atoms, in the alkyl group and 1-4 ethylene oxide units per molecule, which have an HLB value of more than 1.0 and less than/equal to 7.0,
glycerine monoethers of saturated and/or unsaturated, branched and/or unbranched alcohols of a chain length of 8-30, in particular 12-18 carbon atoms,
partial esters of polyglycerines with n=2 to 10 glycerine units and esterified with 1 to 5 saturated or unsaturated, linear or branched, optionally hydroxylated C₈-C₃₀ fatty acid residues, in so far as they have an HLB value of less than/equal to 7,
as well as mixtures of the above cited substances.

6. The stick according to claim 1, wherein the water-in-oil emulsifier c) is selected from ethylene glycol monostearate, ethylene glycol distearate as well as mixtures of both of these substances, also selected from pentaerythrityl monostearate, pentaerythrityl distearate, pentaerythrityl tristearate and pentaerythrityl tetrastearate as well as mixtures of these pentaerythrityl esters, also selected from mixtures of all these substances.

7. The stick according to claim 1, wherein the oil d) that is liquid under normal conditions is selected from
benzoic acid esters of linear or branched C₈₋₂₂ alkanols,
branched saturated or unsaturated fatty alcohols containing 6-30 carbon atoms.
triglycerides of linear or branched, saturated or unsaturated, optionally hydroxylated C₈₋₃₀ fatty acids,
dicarboxylic acid esters of linear or branched C₂-C₁₀ alkanols,
esters of branched saturated or unsaturated fatty alcohols containing 2-30 carbon atoms with linear or branched saturated or unsaturated fatty acids containing 2-30 carbon atoms, which can be hydroxylated,
addition products of 1 to 5 propylene oxide units onto monohydric or polyhydric C₈₋₂₂ alkanols,
addition products of at least 6 ethylene oxide and/or propylene oxide units onto monohydric or polyhydric C₃₋₂₂ alkanols,
C₈-C₂₂ fatty alcohol esters of monohydric or polyhydric C₂-C₇ hydroxycarboxylic acids,
symmetrical, unsymmetrical or cyclic esters of carbonic acid with fatty alcohols,
the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols,
as well as mixtures of the above cited substances.

8. The stick according to claim 1, wherein the oils d) that are liquid under normal conditions comprise maximum 20 wt. % of oil(s), based on the total weight of the oils that are liquid at 20° C., whose solubility parameter differs by more than −1.0 (cal/cm³)^{0.5} or by more than +1.0 (cal/cm³)^{0.5} from the (average) solubility parameter of the water-in-oil emulsifier/of the water-in-oil emulsifiers.

9. The stick according to claim 8, wherein no oils that are liquid under normal conditions are comprised, whose solubility parameter differs by more than ±1.2 (cal/cm³)^{0.5} from the (average) solubility parameter of the water-in-oil emulsifier/of the water-in-oil emulsifiers.

10. The stick according to claim 1, wherein the water-soluble polyhydric C₂-C₈ alkanol e) containing 2-6 hydroxyl groups is selected from 1,2-propylene glycol, 2-methyl-1,3-propanediol, glycerine, butylene glycols such as 1,2-butylene glycol, 1,3-butylene glycol and 1,4-butylene glycol, pentylene glycols such as 1,2-pentanediol and 1,5-pentanediol, hexanediols such as 1,6-hexanediol, hexanetriols such as 1,2,6-hexanetriol, 1,2-octanediol, 1,8-octanediol, di-propylene glycol, tri-propylene glycol, di-glycerine, tri-glycerine, erythritol, sorbitol, xylitol as well as mixtures of the above cited substances.

11. The stick according to claim 1, wherein at least one wax component with a melting point in the range of 25-<50° C. is additionally comprised, selected from coconut fatty acid glycerine mono-, di- and tri-esters, Butyrospermum parkii (Shea Butter) and esters of saturated, monohydric C₈-C₁₈ alcohols with saturated C₁₂-C₁₈ monocarboxylic acids, as well as mixtures of these substances.

12. The stick according to claim 1, wherein the cosmetic or dermatological active substance(s) is/are selected from deodorant and/or antiperspirant active substances, preferably from antiperspirant active substances.

13. The stick composition according to claim 1, having a penetration force value in the range of 150-800 gram force (g-force), 250-700 gram force (g-force), particularly preferably 350-650 gram force (g-force), at a penetration depth of 5.000 mm.

14. The stick composition according to claim 1, having an electrical resistance of a maximum of 400 kΩ, preferably a maximum of 350 kΩ and particularly preferably a maximum of 300 kΩ.

15. The stick according to claims 12, wherein the deodorant active substance is chosen from
arylsulfatase inhibitors, β-glucuronidase inhibitors, aminoacylase inhibitors, esterase inhibitors, lipase inhibitors and lipoxigenase inhibitors,
α-monoalkyl glycerine ethers with a branched or linear saturated or unsaturated, optionally hydroxylated C₆-C₂₂-alkyl residue, in particular α-(2-ethylhexyl) glycerine ether,
phenoxyethanol,
perfume oils with a germ inhibiting action
deosafe perfume oils
prebiotically active components,
trialkyl citric acid esters, in particular triethyl citrate,
active substances, which reduce the number of the odor-causing skin bacteria of the group of the staphylococci, coryne bacteria, anaerococci and micrococci, or which inhibit their growth,
zinc compounds, in particular zinc phenolsulfonate and zinc ricinoleate,
organo-halogen compounds, in particular Triclosan, Chlorhexidine, Chlorhexidine gluconate and Benzalkonium halides,
quaternary ammonium compounds, in particular cetylpyridinium chloride,
odor absorbers, in particular silicates and zeolites,
sodium bicarbonate,
lantibiotics,
as well as mixtures of the above cited substances,
and/or the antiperspirant active substances selected from the water-soluble astringent inorganic and organic salts of aluminum, zirconium and zinc or any mixtures of these salts.

16. The stick according to claim 1, wherein the total content of non-ionic and ionic emulsifiers and/or surfactants with an HLB value of over 8 is maximum 20 wt. %, preferably a maximum of 15 wt. %, particularly preferably a maximum of 10 wt. %, particularly preferably a maximum of 7 wt. %, further particularly preferably a maximum of 4 wt. % and exceedingly preferably a maximum of 3 wt. %, relating respectively to the total composition.

17. A cosmetic, non-therapeutic process for diminishing body odor, wherein a cosmetic stick composition according to claim 1, and which comprises at least one cosmetic or dermatological active substance selected from deodorant and/or antiperspirant active substances, is applied onto the skin.

18. A cosmetic, non-therapeutic process for putting on makeup and/or matting and/or for putting on matting makeup on the skin and/or the mucosa, wherein a cosmetic composition according to claim 1 is applied onto the skin or mucosa.

19. A cosmetic, non-therapeutic process for putting on makeup and/or matting and/or for concealing tiny wrinkles, wrinkles or fine lines, wherein a cosmetic composition according to claim 1 is applied onto the skin or mucosa.

20. A process for manufacturing a stick according to claim 1, wherein the wax and oil components are heated together with the oil-in-water emulsifier(s) and the water-in-oil emulsifier(s) to 90-95° C. and melted, the water, likewise heated to 90-95° C. and containing the water-soluble active substances and ingredients, is then added with vigorous stirring, optional additional ingredients are mixed in, the mixture is then cooled to a suitable filing temperature, filled into suitable dispensing forms and allowed to solidify at room temperature by static cooling without further stirring.
